(19) United States
(12) Patent Application Publication
(10) Pub. No.: US 2015/0315212 A1
(43) Pub. Date: Nov. 5, 2015

(54) SUBSTITUTED 1,4-DITHIINE DERIVATIVES AND THEIR USE AS FUNGICIDES

(71) Applicant: BASF SE, Ludwigshafen (DE)

(72) Inventors: Wassilios GRAMMENOS, Ludwigshafen (DE); Nadege BOUDET, Hemsbach (DE); Erica May Wilson LAUTERWASSER, Wachenheim (DE); Jan Klaas LOHMANN, Lambsheim (DE); Thomas GROTE, Wachenheim (DE); Egon HADEN, Speyer (DE); Bernd MUELLER, Frankenthal (DE); Marcus FEHR, Speyer (DE)

(73) Assignee: BASF SE, Ludwigshafen (DE)

(21) Appl. No.: 14/649,053

(22) PCT Filed: Nov. 25, 2013
(86) PCT No.: PCT/EP2013/074529

§ 371 Date: Jun. 2, 2015

(30) Foreign Application Priority Data
Dec. 4, 2012 (EP) 12195501.7

(51) Int. Cl. C07D 513/14; A01N 43/90

(52) U.S. Cl. CPC C07D 513/14; A01N 43/90

(57) ABSTRACT

The present invention relates to substituted 1,4-dithiine derivatives of formula I as defined in the description, and the N-oxides, and salts thereof, their preparation and intermediates for preparing them. The invention also relates to the use of these compounds for combating harmful fungi and seed coated with at least one such compound and also to compositions comprising at least one such compound.

The present invention relates to compounds of the formula I

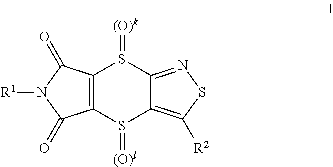

and the N-oxides and the agriculturally acceptable salts thereof.

Furthermore the present invention relates to a process for preparing compounds of the formula I.

Furthermore the present invention relates to agrochemical compositions, comprising an auxiliary and at least one compound of formula I an N-oxide or an agriculturally acceptable salt thereof.

Furthermore the present invention relates to the use of a compound of the formula I and/or of an agriculturally acceptable salt thereof or of the compositions for combating phytopathogenic fungi.

Furthermore the present invention relates to a method for combating harmful fungi, comprising treating the fungi or the materials, plants, the soil or seeds to be protected against fungal attack with an effective amount of at least one compound of formula I or with a composition.

Furthermore the present invention relates to seed, coated with at least one compound of the formula I and/or an agriculturally acceptable salt thereof or with a composition in an amount of from 0.1 to 10 kg per 100 kg of seed.

Compounds of the following formula

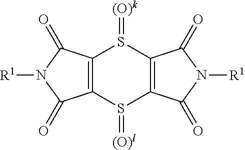

process for their preparation and their application in crop protection are known and described e.g. in WO 2010/043319.

WO 2011/029551 relates to the mixtures of the formula (I)

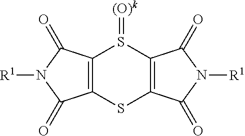

The compounds according to the present invention differ from those described in the abovementioned publications inter alia by the substitution of the 1,4-dithiine ring by the isothiazol ring. In many cases, in particular at low application rates, the fungicidal activity of the known fungicidal compounds is unsatisfactory. Based on this, it was an object of the present invention to provide compounds having improved activity and/or a broader activity spectrum against phytopathogenic harmful fungi.

Accordingly, it is an object of the present invention to provide compounds having better fungicidal activity and/or better crop plant compatibility.

Surprisingly, these objects are achieved by compounds of the general formula I, as defined below, and by the agriculturally acceptable salts of the compounds of the general formula I.

Accordingly, the present invention relates to compounds of formula I

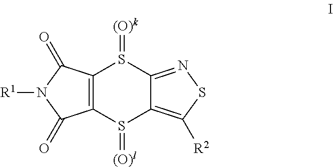

in which
k is 0, 1 or 2; or
l is 0, 1 or 2; or
R¹ is H, OH, halogen, CN, NO₂; or
C₁-C₁₀-alkyl, C₁-C₁₀-alkoxy, C₁-C₁₀-haloalkyl, C₂-C₁₀-alkenyl, C₂-C₁₀-alkynyl, C₃-C₁₀-cycloalkyl, C₃-C₁₀-halocycloalkyl, C₃-C₁₀-cycloalkenyl; or
phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein
   the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₂-C₁₀-alkenyl, C₂-C₁₀-alkynyl, C₁-C₁₀-alkoxy, C₁-C₁₀-haloalkoxy; or
NA¹A² where
   A¹ and A² independently of one another are hydrogen, C₁-C₁₀-alkyl, C₁-C₁₀-haloalkyl, C₃-C₁₀-cycloalkyl, C₃-C₁₀-halocycloalkyl, C₁-C₁₀-alkoxy-C₁-C₁₀-alkyl, amino-C₁-C₁₀-alkyl, or
      independently of one another are phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein
         the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy; or
OA³ where
   A³ is C₁-C₁₀-alkyl, C₁-C₄-alkylcarbonyl, C₁-C₁₀-haloalkyl, C₂-C₆-alkenyl, C₂-C₆-alkynyl, C₃-C₁₀-cycloalkyl, C₃-C₁₀-halocycloalkyl, C₃-C₆-cycloalkenyl, phenyl, benzyl, naphthyl or a saturated, partially unsaturated or aromatic 5-, 6-, 7-, 8, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein
      the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy;

R² is halogen, CN, NO₂; or
NA¹A²
where
A¹ and A² independently of one another are hydrogen, C₁-C₁₀-alkyl, C₁-C₁₀-haloalkyl, C₃-C₁₀-cycloalkyl, C₃-C₁₀-halocycloalkyl, C₁-C₁₀-alkoxy-C₁-C₁₀-alkyl, amino-C₁-C₁₀-alkyl,
   wherein
   the amino group is substituted by B¹ and B² which are independently of one another hydrogen, C₁-C₁₀-alkyl, C₁-C₁₀-haloalkyl, C₃-C₁₀-cycloalkyl, C₃-C₁₀-halocycloalkyl or B¹ and B² together with the N atom to which these radicals are attached may also form a five-, six-, seven-, eight-, nine- or ten-membered saturated or partially unsaturated ring which, in addition to carbon atoms, may contain one, two or three heteroatoms from the group consisting of O, N and S as ring members;
   or
   independently of one another are phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO groups as ring members,
   wherein the above mentioned groups may carry one, two, three or four identical or different substituents selected from the group consisting of halogen, hydroxyl, cyano, nitro, NH₂, mono-(C₁-C₁₀-alkyl)amino, di-(C₁-C₁₀-alkyl)amino, C₁-C₁₀-alkyl, C₁-C₁₀-haloalkyl, C₁-C₁₀-alkoxy, C₁-C₁₀-haloalkoxy; or
a radical of the formula
—C(═O)A⁴, —OC(═O)A⁴, —C(═O)OA⁴, —NA⁴C(═O)A⁴, —N═OA⁴,
where
A⁴ independently of one another are hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO groups as ring members,
   wherein the above mentioned groups may carry one, two, three or four identical or different substituents selected from the group consisting of halogen, hydroxyl, cyano, nitro, NH₂, mono-(C₁-C₁₀-alkyl)amino, di-(C₁-C₁₀-alkyl)amino, C₁-C₁₀-alkyl, C₁-C₁₀-haloalkyl, C₁-C₁₀-alkoxy, C₁-C₁₀-haloalkoxy; or
—S(O)ₙA⁵, —OS(O)ₙA⁵, —NA⁵S(O)ₙA⁵
where
n=0, 1, 2
A⁵ independently of one another are hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO groups as ring members,
   wherein the above mentioned groups may carry one, two, three or four halogen, hydroxyl, cyano, nitro, NH₂, mono-(C₁-C₁₀-alkyl)amino, di-(C₁-C₁₀-alkyl)amino, C₁-C₁₀-alkyl, C₁-C₁₀-haloalkyl, C₁-C₁₀-alkoxy, C₁-C₁₀-haloalkoxy;
      and the N-oxides and the agriculturally acceptable salts thereof.

Furthermore the present invention provides a process for preparing compounds of the formula I.

Furthermore the present invention provides an agrochemical composition, comprising an auxiliary and at least one compound of formula I an N-oxide or an agriculturally acceptable salt thereof.

Furthermore compounds of the formula I and/or of an agriculturally acceptable salt thereof or of the compositions can be used for combating phytopathogenic fungi.

Furthermore the present invention provides a method for combating harmful fungi, comprising treating the fungi or the materials, plants, the soil or seeds to be protected against fungal attack with an effective amount of at least one compound of formula I or with a composition.

Furthermore the present invention provides seed, coated with at least one compound of the formula I and/or an agriculturally acceptable salt thereof or with a composition in an amount of from 0.1 to 10 kg per 100 kg of seed.

The terms used for organic groups in the definition of the variables are, for example the expression “halogen”, collective terms which represent the individual members of these groups of organic units.

The prefix Cₓ-C_{y} denotes the number of possible carbon atoms in the particular case.

halogen: fluorine, bromine, chlorine or iodine, especially fluorine, chlorine or bromine;
alkyl and the alkyl moieties of composite groups such as, for example, alkoxy, alkylamino, alkoxycarbonyl: saturated straight-chain or branched hydrocarbon radicals having 1 to 10 carbon atoms, for example C₁-C₁₀-akyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; heptyl, octyl, 2-ethylhexyl and positional isomers thereof; nonyl, decyl and positional isomers thereof;
haloalkyl: straight-chain or branched alkyl groups having 1 to 10 carbon atoms (as mentioned above), where some or all of the hydrogen atoms in these groups are replaced by halogen atoms as mentioned above. In one embodiment, the alkyl groups are substituted at least once or completely by a particular halogen atom, preferably fluorine, chlorine or bromine. In a further embodiment, the alkyl groups are partially or fully halogenated by different halogen atoms; in the case of mixed halogen substitutions, the combination of chlorine and fluorine is preferred. Particular preference is given to (C₁-C₃)-haloalkyl, more preferably (C₁-C₂)-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl and also the alkenyl moieties in composite groups, such as alkenyloxy: unsaturated straight-chain or branched hydrocarbon radicals having 2 to 10 carbon atoms and one double bond in any position. According to the invention, it may be preferred to use small alkenyl groups, such as (C₂-C₄)-alkenyl; on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl and the alkynyl moieties in composite groups: straight-chain or branched hydrocarbon groups having 2 to 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl and also the cycloalkyl moieties in composite groups: mono- or bicyclic saturated hydrocarbon groups having 3 to 10, in particular 3 to 6, carbon ring members, for example C₃-C₆-cycloalkyl, such as cyclopropyl, cyclobutyl, cyclopentyl, cyclohexyl, cycloheptyl or cyclooctyl. Examples of bicyclic radicals comprise bicyclo[2.2.1]heptyl, bicyclo[3.1.1]heptyl, bicyclo[2.2.2]octyl and bicyclo[3.2.1]octyl. In this connection, optionally substituted C₃-C₈-cycloalkyl means a cycloalkyl radical having from 3 to 8 carbon atoms, in which at least one hydrogen atom, for example 1, 2, 3, 4 or 5 hydrogen atoms, is/are replaced by substituents which are inert under the conditions of the reaction. Examples of inert substituents are CN, C₁-C₆-alkyl, C₁-C₄-haloalkyl, C₁-C₆-alkoxy, C₃-C₆-cycloalkyl, and C₁-C₄-alkoxy-C₁-C₆-alkyl;
halocycloalkyl and the halocycloalkyl moieties in halocycloalkoxy, halocycloalkylcarbonyl and the like: monocyclic saturated hydrocarbon groups having 3 to 10 carbon ring members (as mentioned above) in which some or all of the hydrogen atoms may be replaced by halogen atoms as mentioned above, in particular fluorine, chlorine and bromine;
cycloalkenyl: monocyclic monounsaturated hydrocarbon groups having 3 to 10, 3 to 8, 3 to 6, preferably 5 to 6, carbon ring members, such as cyclopenten-1-yl, cyclopenten-3-yl, cyclohexen-1-yl, cyclohexen-3-yl, cyclohexen-4-yl and the like;
alkoxy: an alkyl group as defined above which is attached via an oxygen, preferably having 1 to 10, more preferably 2 to 6, carbon atoms. Examples are: methoxy, ethoxy, n-propoxy, 1-methylethoxy, butoxy, 1-methylpropoxy, 2-methylpropoxy or 1,1-dimethylethoxy, and also for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
haloalkoxy: alkoxy as defined above, where some or all of the hydrogen atoms in these groups are replaced by halogen atoms as described above under haloalkyl, in particular by fluorine, chlorine or bromine. Examples are OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;
6- to 10-membered aryl: aromatic cyclus with 6, 7, 8, 9 oder 10 C atoms. Examples of preferred aryl are phenyl or naphthyl;
5-, 6-, 7-, 8-, 9- or 10-membered saturated, partially unsaturated or aromatic heterocycle which contains 1, 2, 3 or 4 heteroatoms from the group consisting of O, N and S as ring members, and may furthermore contain one or two CO, SO, SO₂ groups as ring members, where the heterocycle in question may be attached via a carbon atom or, if present, via a nitrogen atom. In particular:
a five- or six-membered saturated or partially unsaturated heterocycle which comprises one, two, three or four heteroatoms from the group consisting of O, N and S as ring members: for example monocyclic saturated or partially unsaturated heterocycles which, in addition to carbon ring members, comprise one, two or three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals;
a seven-membered saturated or partially unsaturated heterocycle which comprises one, two, three or four heteroatoms from the group consisting of O, N and S as ring members: for example mono- and bicyclic heterocycles having 7 ring members which, in addition to carbon ring members, comprise one, two or three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-,
-3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding ylidene radicals;
a five- or six-membered aromatic heterocycle (=heteroaromatic radical) which contains one, two, three or four heteroatoms from the group consisting of oxygen, nitrogen and sulfur, for example 5-membered heteroaryl which is attached via carbon and contains one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl, which contains one, two or three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;
   C₁-C₁₀-alkoxy-C₁-C₁₀-alkyl: C₁-C₁₀-alkyl (as defined above) where one hydrogen atom is replaced by a C₁-C₁₀-alkoxy group as defined above;
   amino-C₁-C₁₀-alkyl: C₁-C₁₀-alkyl (as defined above) where one hydrogen atom is replaced by a NA¹A² group as defined above;
   mono-(C₁-C₁₀-alkyl)amino: group of the formula NA¹A² group in which A¹ or A² is an C₁-C₁₀-alkyl group as defined above.
   di-(C₁-C₁₀-alkyl)amino: group of the formula NA¹A² group in which each A¹ and A² are an C₁-C₁₀-alkyl group as defined above.
   hydroxyl: OH group which is attached via an O atom;
   cyano: CN group which is attached via an C atom;
   nitro: NO₂ group which is attached via an N atom.

Depending on the substitution pattern, the compounds of the formula I according to the invention may have one or more centers of chirality, and are generally obtained in the form of racemates or as diastereomer mixtures of erythro and threo forms. The erythro and threo diastereomers of the compounds according to the invention can be separated and isolated in pure form, for example, on the basis of their different solubilities or by column chromatography. Using known methods, such uniform pairs of diastereomers can be used to obtain uniform enantiomers. Suitable for use as antimicrobial agents are both the uniform diastereomers or enantiomers and mixtures thereof obtained in the synthesis. This applies correspondingly to the fungicidal compositions.

Accordingly, the invention provides both the pure enantiomers or diastereomers and mixtures thereof. This applies to the compounds of the formula I according to the invention and, if appropriate, correspondingly to their precursors. The scope of the present invention includes in particular the (R) and (S) isomers and the racemates of the compounds according to the invention, in particular of the formula I, which have centers of chirality. Suitable compounds of the formula I according to the invention also comprise all possible stereoisomers (cis/trans isomers) and mixtures thereof.

The compounds according to the invention may be present in various crystal modifications which may differ in their biological activity. They are likewise provided by the present invention.

Owing to the basic character of their nitrogen atoms, the compounds of the formula I according to the invention are capable of forming salts or adducts with inorganic or organic acids or with metal ions.

Suitable agriculturally useful salts are especially the salts of those cations or the acid addition salts of those acids whose cations and anions, respectively, have no adverse effect on the fungicidal action of the compounds of the formula I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can be formed by reacting with an acid of the corresponding anion, preferably hydrochloric acid, hydrobromic acid, sulfuric acid, phosphoric acid or nitric acid.

The inventive compounds can be present in atropisomers arising from restricted rotation about a single bond of asymmetric groups. They also form part of the subject matter of the present invention.

Depending on the substitution pattern, the compounds of formula I and their N-oxides may have one or more centers of chirality, in which case they are present as pure enantiomers or pure diastereomers or as enantiomer or diastereomer compositions. Both, the pure enantiomers or diastereomers and their compositions are subject matter of the present invention.

The compounds of the formula I according to the invention can be prepared by different routes analogously to processes known per se of the prior art (see, for example, the prior art cited at the outset).

The compounds of the formula I according to the invention can be prepared by different routes analogously to processes known per se of the prior art as follows:

The compounds of the formula I-A according to the invention in which k=0 and l=0 can be prepared by different routes analogously to processes known per se of the prior art as follows:

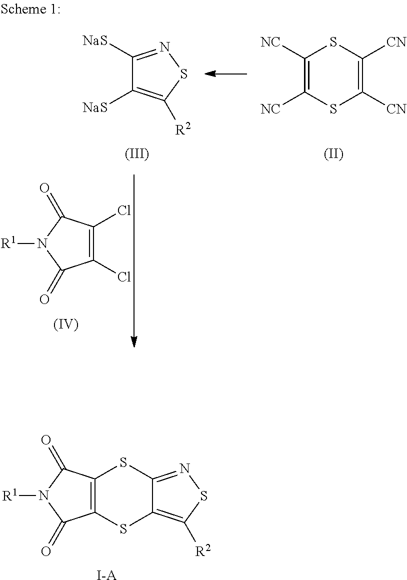

According to the above mentioned route a precursor II can be converted into the corresponding compounds of the formula (III) (for details see: Journal of Organic Chemistry, 45(25), 5122-30; 1980).

This can be coupled with the precursors (IV) providing efficient access to the corresponding compounds of the formula (I-A) (for details see: U.S. Pat. No. 5,633,219).

The compounds of the formulae I-B according to the invention in which k=1 and l=1, I-C in which k=1 and l=2, I-D in which k=2 and l=1, I-E in which k=2 and l=2 can be prepared by different routes analogously to processes known per se of the prior art as follows:

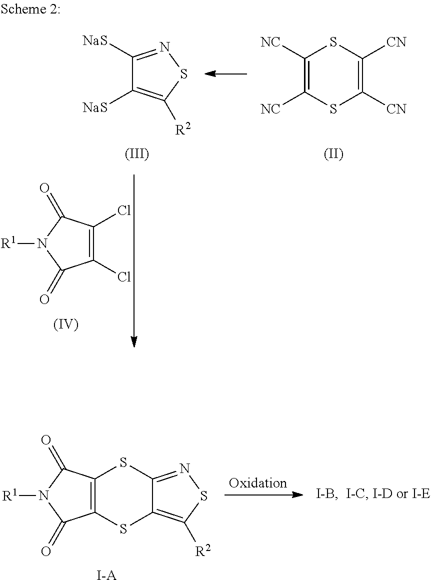

According to the above mentioned route a precursor II can be converted into the corresponding compounds of the formula (VI) (for details see: U.S. Pat. No. 5,633,219) which can be oxided according to conventional oxidation methods, e. g. by treating compounds (VI) with an organic peracid such as metachloroperbenzoic acid (cf. WO 03/64572 or J. Med. Chem. 38(11), 1892-903, 1995) or with inorganic oxidizing agents such as hydrogen peroxide (cf. J. Heterocyc. Chem. 18(7), 1305-8, 1981) or using ammonium cerium(IV) nitrate in the presence of a catalytic amount of KBr or NaBr) (for details see: Catalysis Communications, 9(8), 1739-1744; 2008; Monatshefte fuer Chemie, 139(8), 895-899; 2008) giving the corresponding compounds of the formula (IB), (IC), (ID), (IE).

The precursor (III) may be obtained by various routes in analogy to prior art processes known from JOC, 1980, 45(25), 5122-30.

The precursor (IV) can be prepared employing a preparative procedure known from WO 2012/139987, WO 2012/40001.

If individual inventive compounds cannot be directly obtained by the routes described above, they can be prepared by derivatization of other inventive compounds.

The N-oxides may be prepared from the inventive compounds according to conventional oxidation methods, e. g. by treating compounds I with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocyc. Chem. 18(7), 1305-8, 1981) or oxone (cf. J. Am. Chem. Soc. 123(25), 5962-5973, 2001). The oxidation may lead to pure mono-N-oxides or to a composition of different N-oxides, which can be separated by conventional methods such as chromatography.

If the synthesis yields compositions of isomers, a separation is generally not necessarily required since in some cases the individual isomers can be interconverted during work-up for use or during application (e. g. under the action of light, acids or bases). Such conversions may also take place after use, e. g. in the treatment of plants in the treated plant, or in the harmful fungus to be controlled.

In the compounds of the formula I according to the invention particular preference is given to the following meanings of the substituents, in each case on their own or in combination.

k in the compounds according to the invention is, according to one embodiment, 0. k in the compounds according to the invention is, according to one further embodiment, 1. k in the compounds according to the invention is, according to one further embodiment, 2.

l in the compounds according to the invention is, according to one embodiment, 0. l in the compounds according to the invention is, according to one further embodiment, 1. l in the compounds according to the invention is, according to one further embodiment, 2.

R¹ in the compounds according to the invention is, according to one embodiment, H, OH, halogen, cyano, nitro. In a special embodiment of the invention, R¹ is H. In a further special embodiment of the invention, R¹ is OH. In a further special embodiment of the invention, R¹ is CN. In a further special embodiment of the invention, R¹ is nitro. In a further special embodiment of the invention, R¹ is Cl or F.

R¹ in the compounds according to the invention is, according to a further embodiment, C₁-C₁₀-alkyl, C₁-C₁₀-alkoxy, C₁-C₁₀-haloalkyl, C₂-C₁₀-alkenyl, C₂-C₁₀-alkynyl, C₃-C₁₀-cycloalkyl, C₃-C₁₀-halocycloalky or C₃-C₁₀-cycloalkenyl.

According to one embodiment R¹ is C₁-C₁₀-alkyl, preferably methyl, ethyl, n-propyl, i-propyl, n-butyl, i-butyl, t-butyl, in particular methyl, ethyl, wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₂-C₁₀-alkenyl, C₂-C₁₀-alkynyl, C₁-C₁₀-alkoxy, C₁-C₁₀-haloalkoxy, OA³, —C(═O)A⁴, —C(═O)OA⁴ in particular C₂-C₁₀-alkenyl, C₂-C₁₀-alkynyl, OA³, —C(═O)A⁴, wherein OA³, —C(═O)A⁴ are as defined below. In a special embodiment of the invention, R¹ is methyl. In a further special embodiment of the invention, R¹ is ethyl. In a further special embodiment of the invention, R¹ is propyl. In a further special embodiment R¹ is i-propyl. In a further special embodiment R¹ is 1-methylpropyl. In a further special embodiment R¹ is n-butyl. In a further special embodiment R¹ is i-butyl. In a further special embodiment R¹ is t-butyl. In a further special embodiment R¹ is n-pentyl. In a further special embodiment R¹ is n-hexyl.

According to a further embodiment R¹ is C₁-C₁₀-haloalkyl; preferrably fully or partially halogenated methyl, ethyl, n-propyl, i-propyl, n-butyl, i-butyl, t-butyl, in particular fully or partially halogenated methyl. In a special embodiment of the invention, R¹ is CF₃. In a further special embodiment of the invention, R¹ is CHF₂. In a further special embodiment R¹ is CFH₂. In a further special embodiment of the invention, R¹ is CH₂CF₃. In a further special embodiment R¹ is CCl₃. In a further special embodiment R¹ is CHCl₂. In a further special embodiment R¹ is CClH₂.

According to a further embodiment R¹ is C₁-C₁₀-alkoxy. In a special embodiment of the invention, R¹ is OCH₃. In a further special embodiment of the invention, R¹ is OCH₂CH₃. In a further special embodiment of the invention, R¹ is OCH₂CH₂CH₃. In a further special embodiment of the invention, R¹ is OCH(CH₃)₂. In a further special embodiment of the invention, R¹ is OCH₂CH₂CH₂CH₃. In a further special embodiment of the invention, R¹ is OCH(CH₂CH₃)₂. In a further special embodiment of the invention, R¹ is OC(CH₃)₃. In a further special embodiment of the invention, R¹ is OCH₂CH(CH₃)₂. In a further special embodiment of the invention, R¹ is OCH₂CH₂CH₂CH₂CH₃. In a further special embodiment of the invention, R¹ is O CH₂CH₂CH₂CH₂CH₂CH₃.

According to a further embodiment R¹ is C₂-C₁₀-alkenyl, preferably C₂-C₄-alkenyl. In a special embodiment of the invention, R¹ is vinyl. In a special embodiment R¹ is CH═CH₂. In a further special embodiment R¹ is CH₂CH═CH₂. In a further special embodiment R¹ is CH₂C(CH₃)═CH₂. In a further special embodiment R¹ is CH₂C(CH₃)═CHCH₃. In a further special embodiment R¹ is CH₂C(CH₃)═C(CH₃)₂. In a further special embodiment R¹ is CH₂CH═CHCH₃. In a further special embodiment R¹ is CH═CHCH₃. In a further special embodiment R¹ is CH₂C(CH₃)═CH₂.

According to a further embodiment R¹ is C₂-C₁₀-alkynyl, preferably C₂-C₄-alkynyl. In a special embodiment of the invention, R¹ is ethynyl. In a further special embodiment of the invention, R¹ is 1-propynyl. In a further special embodiment of the invention, R¹ is 2-propynyl. In a further special embodiment of the invention, R¹ is 1-butynyl. In a further special embodiment of the invention, R¹ is 3-methyl-but-1-ynyl.

According to a further embodiment R¹ is C₃-C₁₀-cycloalkyl, preferably is C₃-C₆-cycloalkyl. In a special embodiment of the invention, R¹ is cyclopropyl. In a further special embodiment of the invention, R¹ is cyclopentyl.

According to a further embodiment R¹ is C₃-C₁₀-halocycloalkyl, preferrably fully or partially halogenated cyclopropyl, cyclobutyl, cyclopentyl, in particular fully or partially halogenated cyclopropyl. In a further special embodiment R¹ is 1-Cl-cyclopropyl. In a further special embodiment R¹ is 2-Cl-cyclopropyl. In a further special embodiment R¹ is 1-F-cyclopropyl. In a further special embodiment R¹ is 2-F-cyclopropyl. In a further special embodiment R¹ is fully or partially halogenated cyclobutyl. In a further special embodiment R¹ is 1-Cl-cyclobutyl. In a further special embodiment R¹ is 1-F-cyclobutyl. In a further special embodiment R¹ is 2-Cl-cyclobutyl. In a further special embodiment R¹ is 3-Cl-cyclobutyl. In a further special embodiment R¹ is 2-F-cyclobutyl. In a further special embodiment R¹ is 3-F-cyclobutyl. In a further special embodiment R1 is 3,3-(Cl)₂-cyclobutyl. In a further special embodiment R1 is 3,3-(F)₂-cyclobutyl. According to a further embodiment R¹ is C₃-C₁₀-cycloalkenyl, preferably cyclopropenyl.

In a specific embodiment of the invention R¹ is CH₂CHO, CH₂C(═O)OH, CH₂C(═O NH₂CH₂C(═O)OC₁-C₁₀-alkyl, CH₂C(═O)—NHC₁-C₁₀-alkyl, CH₂C(═O)—N(C₁-C₁₀-alkyl)₂, CH(C₁-C₄-alkyl)-CHO, CH(C₁-C₄-alkyl)-C(═O)OH, CH(C₁-C₄-alkyl)-C(═O)NH₂CH(C₁-C₄-alkyl)-C(═O)OC₁-C₁₀-alkyl, CH(C₁-C₄-alkyl)-C(═O)NHC₁-C₁₀-alkyl, CH(C₁-C₄-alkyl)-C(═O)N(C₁-C₁₀-alkyl)₂, C(C₁-C₄-alkyl)₂-CHO, C(C₁-C₄-alkyl)₂-C(═O)OH, C(C₁-C₄-alkyl)₂-C(═O)NH₂C(C₁-C₄-alkyl)₂-C(═O)OC₁-C₁₀-alkyl, C(C₁-C₄-alkyl)₂-C(═O)NHC₁-C₁₀-alkyl, C(C₁-C₄-alkyl)₂-C(═O)N(C₁-C₁₀-alkyl)₂R¹ in the compounds according to the invention is, according to a further embodiment, phenyl, benzyl, naphthyl, a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members.

According to one embodiment R¹ is phenyl. According to a further embodiment R¹ is benzyl. According to a further embodiment R¹ is naphthyl. According to one embodiment R¹ is a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members. In a special embodiment of the invention, R¹ is phenyl or benzyl.

According to one embodiment phenyl, benzyl, naphthyl and the saturated, partially unsaturated or aromatic 5-, 6-, 7-, 8-, 9- or 10-membered heterocycle may carry one, two, three or four identical or different substituents selected from the group consisting of halogen, hydroxyl, cyano, nitro, NH₂, C₁-C₁₀-alkyl, C₁-C₁₀-haloalkyl, C₂-C₁₀-alkenyl, C₂-C₁₀-alkynyl, C₁-C₁₀-alkoxy, C₁-C₁₀-haloalkoxy, are as defined below. According to a further embodiment the above mentioned groups may carry one, two, three or four identical or different substituents selected from the group consisting Cl, I, F, Br, preferably Cl, F. According to a further embodiment the above mentioned groups may carry one, two, three or four identical or different substituents selected from the group consisting of methyl, ethyl, propyl, butyl, preferably methyl. According to a further embodiment the above mentioned groups may carry one, two, three or four identical or different substituents selected from the group consisting of partially or fully halogenated methyl, ethyl, propyl, butyl, preferably partially or fully halogenated methyl, in particular CF₃. According to a further embodiment the above mentioned groups may carry one, two, three or four identical or different substituents selected from the group consisting of methoxy, ethoxy, propoxy, butoxy, preferably methoxy. According to a further embodiment the above mentioned groups may carry one, two, three or four identical or different substituents selected from the group consisting of partially or fully halogenated methoxy, ethoxy, propoxy, butoxy, preferably partially or fully halogenated methoxy, in particular OCF₃.

In a special embodiment of the invention, R¹ is phenyl. In a further special embodiment of the invention, R¹ is phenyl substituted by Cl, F, Br, I, CH₃, OCH₃, CF₃ or OCF₃, especially 2,4-dichlorophenyl or 4-chlorophenyl. In a further special embodiment of the invention, R¹ is benzyl substituted by Cl, F, Br, I, CH₃, OCH₃, CF₃ or OCF₃. In a further special embodiment of the invention, R¹ is naphthyl substituted by Cl, F, Br, I, CH₃, OCH₃, CF₃ or OCF₃. In the compounds according to the invention particular preference is given to the following meanings of the substituent R¹, in each case on their own or in combination:
2-chlorophenyl, 2-fluorophenyl, 2-methylphenyl, 2-trifluoromethylphenyl;
3-chlorophenyl, 3-fluorophenyl, 3-methylphenyl, 3-trifluoromethylphenyl;
4-chlorophenyl, 4-fluorophenyl, 4-methylphenyl, 4-trifluoromethylphenyl;
2-chloro-4-fluorophenyl, 2,4-dichlorophenyl, 2,4-difluorophenyl, 2-fluoro-4-chlorophenyl;
3-chloro-4-fluorophenyl, 3,4-dichlorophenyl, 3,4-difluorophenyl, 3-fluoro-4-chlorophenyl;
3-fluoro-5-chlorophenyl or 3,5-difluorophenyl.

R¹ in the compounds according to the invention is, according to a further embodiment, NA¹A² where A¹ and A² independently of one another are hydrogen, C₁-C₁₀-alkyl, C₁-C₁₀-haloalkyl, C₃-C₁₀-cycloalkyl, C₃-C₁₀-halocycloalkyl, C₁-C₁₀-alkoxy-C₁-C₁₀-alkyl, amino-C₁-C₁₀-alkyl; or independently of one another are phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy.

According to one embodiment A¹ and A² independently of one another are hydrogen. According to a further embodiment A¹ and A² independently of one another are hydrogen or C₁-C₁₀-alkyl, preferably methyl, ethyl, n-propyl, i-propyl, n-butyl, i-butyl, t-butyl. In a special embodiment of the invention, R¹ is NH₂, NHCH₃, N(CH₃)₂, NHC₂H₅, NHn-C₃H₇, NHi-C₃H₇, NHn-Calls, NHi-C₄H₉, NHt-C₄H₉. According to a further embodiment A¹ and A² independently of one another are hydrogen or C₃-C₁₀-cycloalkyl, preferably cyclopropyl, cyclobutyl, cyclopenyl, cyclohexyl. In a special embodiment of the invention, R¹ is cyclopropylamino, cyclobutylamino, cyclopentylamino, cyclohexylamino. According to a further embodiment A¹ and A² independently of one another are C₁-C₁₀-alkoxy-C₁-C₁₀-alkyl, preferably C₁-C₄-alkoxy-C₁-C₄-alkyl. In a special embodiment of the invention, R¹ is methoxyethylamino, methoxypropylamino, methoxybutylamino, ethoxyethylamino, ethoxypropylamino or ethoxybutylamino. In a special embodiment of the invention, R¹ is NH₂. In a further special embodiment of the invention, R¹ is N(CH₃)₂. In a further special embodiment of the invention, R¹ is N(C₂H₅)₂.

According to one embodiment A¹ and A² independently of one another are hydrogen or phenyl. According to one embodiment A¹ and A² independently of one another are hydrogen or benzyl. According to a further embodiment A¹ and A² independently of one another are hydrogen or naphthyl. According to a further embodiment A¹ and A² independently of one another are hydrogen or a saturated partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO, SO₂ groups as ring members. In a special embodiment of the invention, R¹ is NH₂. In a further special embodiment of the invention, R¹ is N(phenyl)₂. In a further special embodiment of the invention, R¹ is N(benzyl)₂.

According to one embodiment phenyl, benzyl, naphthyl and the saturated, partially unsaturated or aromatic 5-, 6-, 7-, 8-, 9- or 10-membered heterocycle may carry one, two, three or four identical or different substituents selected from the group consisting of Cl, I, F, Br, preferably Cl, F. According to a further embodiment the above mentioned groups may carry one, two, three or four identical or different substituents selected from the group consisting of methyl, ethyl, propyl, butyl, preferably methyl. According to a further embodiment the above mentioned groups may carry one, two, three or four identical or different substituents selected from the group consisting of partially or fully halogenated methyl, ethyl, propyl, butyl, preferably partially or fully halogenated methyl, in particular CF₃. According to a further embodiment the above mentioned groups may carry one, two, three or four identical or different substituents selected from the group consisting of methoxy, ethoxy, propoxy, butoxy, preferably methoxy. According to a further embodiment the above mentioned groups may carry one, two, three or four identical or different substituents selected from the group consisting of partially or fully halogenated methoxy, ethoxy, propoxy, butoxy, preferably partially or fully halogenated methoxy, in particular OCF₃. In a special embodiment of the invention, A¹ and/or A² are phenyl. In a further special embodiment of the invention, A¹ and/or A² are phenyl substituted by Cl, F, Br, I, CH₃, OCH₃, CF₃ or OCF₃. In a further special embodiment of the invention, A¹ and/or A² are benzyl substituted by Cl, F, Br, I, CH₃, OCH₃, CF₃ or OCF₃. In a further special embodiment of the invention, A¹ and/or A² are naphthyl substituted by Cl, F, Br, I, CH₃, OCH₃, CF₃ or OCF₃.

R¹ in the compounds according to the invention is, according to a further embodiment, OA³, where A³ is hydrogen, C₁-C₁₀-alkyl, C₁-C₁₀-haloalkyl, C₃-C₁₀-cycloalkyl, C₃-C₁₀-halocycloalkyl, phenyl, benzyl, naphthyl or a saturated, partially unsaturated or aromatic 5-, 6-, 7-, 8, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members.

According to one embodiment A³ is H or C₁-C₁₀-alkyl, preferably methyl, ethyl, n-propyl, i-propyl, n-butyl, i-butyl or t-butyl. In a special embodiment of the invention, R¹ is OCH₃, OC₂H₅, On-C₃H₇, Oi-C₃H₇, On-C₄H₉ or Oi-C₄H₉, Ot-C₄H₉. According to a further embodiment A³ is C₁-C₁₀-haloalkyl; preferrably fully or partially halogenated methyl, ethyl, n-propyl, i-propyl, n-butyl, i-butyl or t-butyl, in particular fully or partially halogenated methyl. According to a further embodiment A³ is C₂-C₁₀-alkenyl, preferably ethylene. According to a further embodiment A³ is C₂-C₁₀-alkynyl, preferred ethynyl or 1-propynyl. According to a further embodiment A³ is C₃-C₁₀-cycloalkyl, preferrably cyclopropyl, cyclobutyl or cyclopentyl, in particular cyclopropyl or cyclobutyl. According to a further embodiment A³ is C₃-C₁₀-halocycloalkyl, preferrably fully or partially halogenated cyclopropyl, cyclobutyl or cyclopentyl, in particular fully or partially halogenated cyclopropyl. According to a further embodiment A³ is C₃-C₁₀-cycloalkenyl, preferably cyclopropenyl.

According to one embodiment A³ is phenyl. According to a further embodiment A³ is benzyl. According to a further embodiment A³ is naphthyl. According to one embodiment A³ is a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members.

According to one embodiment phenyl, benzyl, naphthyl and the saturated, partially unsaturated or aromatic 5-, 6-, 7-, 8-, 9- or 10-membered heterocycle may carry one, two, three or four identical or different substituents selected from the group consisting of Cl, I, F, Br, preferably Cl, F. According to a further embodiment the above mentioned groups may carry one, two, three or four identical or different substituents selected from the group consisting of methyl, ethyl, propyl, butyl, preferably methyl. According to a further embodiment the above mentioned groups may carry one, two, three or four identical or different substituents selected from the group consisting of partially or fully halogenated methyl, ethyl, propyl, butyl, preferably partially or fully halogenated methyl, in particular CF₃. According to a further embodiment the above mentioned groups may carry one, two, three or four identical or different substituents selected from the group consisting of methoxy, ethoxy, propoxy, butoxy, preferably methoxy. According to a further embodiment the above mentioned groups may carry one, two, three or four identical or different substituents selected from the group consisting of partially or fully halogenated methoxy, ethoxy, propoxy, butoxy, preferably partially or fully halogenated methoxy, in particular OCF₃. In a special embodiment of the invention, R¹ is OPh, wherein phenyl can be substituted by Cl, F, Br, I, CH₃, OCH₃, CF₃ or OCF₃.

R¹ in the compounds according to the invention is, according to a further embodiment, a radical of the formula —C(═O)A⁴, —C(═O)OA⁴, —OC(═O)A⁴, —NA⁴C(═O)A⁴, —NA⁴C(═O)OA⁴ -N═OA⁴ where A⁴ is hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy.

According to one embodiment A⁴ is H or C₁-C₁₀-alkyl, preferably methyl, ethyl, n-propyl, i-propyl, n-butyl, i-butyl or t-butyl. According to a further embodiment A⁴ is C₁-C₁₀-haloalkyl; preferrably fully or partially halogenated methyl, ethyl, n-propyl, i-propyl, n-butyl, i-butyl or t-butyl, in particular fully or partially halogenated methyl. According to a further embodiment A⁴ is C₂-C₁₀-alkenyl, preferably ethylene. According to a further embodiment A⁴ is C₂-C₁₀-alkynyl, preferred ethynyl or 1-propynyl. According to a further embodiment A⁴ is C₃-C₁₀-cycloalkyl, preferrably cyclopropyl, cyclobutyl or cyclopentyl, in particular cyclopropyl or cyclobutyl. According to a further embodiment A⁴ is C₃-C₁₀-halocycloalkyl, preferrably fully or partially halogenated cyclopropyl, cyclobutyl or cyclopentyl, in particular fully or partially halogenated cyclopropyl. According to a further embodiment A⁴ is C₃-C₁₀-cycloalkenyl, preferably cyclopropenyl. According to a further embodiment A⁴ is NH₂. According to a further embodiment A⁴ is mono-(C₁-C₁₀-alkyl)amino. According to a further embodiment A⁴ is di-(C₁-C₁₀-alkyl)amino.

According to one embodiment A⁴ is phenyl. According to a further embodiment A⁴ is benzyl. According to a further embodiment A⁴ is naphthyl. According to one embodiment A⁴ is a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members.

According to one embodiment phenyl, benzyl, naphthyl and the saturated, partially unsaturated or aromatic 5-, 6-, 7-, 8-, 9- or 10-membered heterocycle may carry one, two, three or four identical or different substituents selected from the group consisting of Cl, I, F, Br, preferably Cl, F. According to a further embodiment the above mentioned groups may carry one, two, three or four identical or different substituents selected from the group consisting of methyl, ethyl, propyl, butyl, preferably methyl. According to a further embodiment the above mentioned groups may carry one, two, three or four identical or different substituents selected from the group consisting of partially or fully halogenated methyl, ethyl, propyl, butyl, preferably partially or fully halogenated methyl, in particular CF₃. According to a further embodiment the above mentioned groups may carry one, two, three or four identical or different substituents selected from the group consisting of methoxy, ethoxy, propoxy, butoxy, preferably methoxy. According to a further embodiment the above mentioned groups may carry one, two, three or four identical or different substituents selected from the group consisting of partially or fully halogenated methoxy, ethoxy, propoxy, butoxy, preferably partially or fully halogenated methoxy, in particular OCF₃.

In a specific embodiment of the invention R¹ is CHO, C(═O)OH, C(═O)NH₂C(═O)OC₁-C₁₀-alkyl, C(═O)NHC₁-C₁₀-alkyl, C(═O)N(C₁-C₁₀-alkyl)₂.

Further in a specific embodiment of the invention R¹ is NH—C(═O)OH, NH—C(═O)NH₂NH—C(═O)OC₁-C₁₀-alkyl, NH—C(═O)—NHC₁-C₁₀-alkyl, NH—C(═O)—N(C₁-C₁₀-alkyl)₂, N(CH₃)C(═O)OH, N(CH₃)—C(═O)NH₂N(CH₃)—C(═O)OC₁-C₁₀-alkyl, N(CH₃)—C(═O)—NHC₁-C₁₀-alkyl, N(CH₃)—C(═O)—N(C₁-C₁₀-alkyl)₂.

R¹ in the compounds according to the invention is, according to a further embodiment, a radical of the formula —S(O)ₙA⁵, —OS(O)ₙA⁵, —NA⁵S(O)ₙA⁵ where n=0, 1, 2 and A⁵ independently of one another are hydrogen, hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated or unsaturated aromatic or non-aromatic 5-, 6-, 7-, 8, 9- or 10-membered heterocycle, where the heterocycle contains 1, 2, 3 or 4 heteroatoms selected from the group consisting of O, N and S as ring members and may furthermore contain one or two CO groups as ring members, wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy.

According to one embodiment A⁵ is H or C₁-C₁₀-alkyl, preferably methyl, ethyl, n-propyl, i-propyl, n-butyl, i-butyl or t-butyl. According to a further embodiment A⁵ is C₁-C₁₀-haloalkyl; preferrably fully or partially halogenated methyl, ethyl, n-propyl, i-propyl, n-butyl, i-butyl or t-butyl, in particular fully or partially halogenated methyl. According to a further embodiment A⁵ is C₂-C₁₀-alkenyl, preferably ethylene. According to a further embodiment A⁵ is C₂-C₁₀-alkynyl, preferred ethynyl or 1-propynyl. According to a further embodiment A⁵ is C₃-C₁₀-cycloalkyl, preferrably cyclopropyl, cyclobutyl or cyclopentyl, in particular cyclopropyl or cyclobutyl. According to a further embodiment A⁵ is C₃-C₁₀-halocycloalkyl, preferrably fully or partially halogenated cyclopropyl, cyclobutyl or cyclopentyl, in particular fully or partially halogenated cyclopropyl. According to a further embodiment A⁵ is C₃-C₁₀-cycloalkenyl, preferably cyclopropenyl. According to a further embodiment A⁵ is NH₂. According to a further embodiment A⁵ is mono-(C₁-C₁₀-alkyl)amino. According to a further embodiment A⁵ is di-(C₁-C₁₀-alkyl)amino.

In a specific embodiment of the invention R¹ is SH and S—C₁-C₁₀-alkyl.

According to one embodiment A⁵ is phenyl. According to a further embodiment A⁵ is benzyl. According to a further embodiment A⁵ is naphthyl. According to one embodiment A⁵ is a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members.

According to one embodiment phenyl, benzyl, naphthyl and the saturated, partially unsaturated or aromatic 5-, 6-, 7-, 8-, 9- or 10-membered heterocycle may carry one, two, three or four identical or different substituents selected from the group consisting of Cl, I, F, Br, preferably Cl, F. According to a further embodiment the above mentioned groups may carry one, two, three or four identical or different substituents selected from the group consisting of methyl, ethyl, propyl, butyl, preferably methyl. According to a further embodiment the above mentioned groups may carry one, two, three or four identical or different substituents selected from the group consisting of partially or fully halogenated methyl, ethyl, propyl, butyl, preferably partially or fully halogenated methyl, in particular CF₃. According to a further embodiment the above mentioned groups may carry one, two, three or four identical or different substituents selected from the group consisting of methoxy, ethoxy, propoxy, butoxy, preferably methoxy. According to a further embodiment the above mentioned groups may carry one, two, three or four identical or different substituents selected from the group consisting of partially or fully halogenated methoxy, ethoxy, propoxy, butoxy, preferably partially or fully halogenated methoxy, in particular OCF₃.

R² in the compounds according to the invention is, according to one embodiment, halogen, CN, NO₂. In a further special embodiment of the invention, R² is halogen such as Cl or F. In a further special embodiment of the invention, R² is CN. In a further special embodiment of the invention, R² is nitro.

R² in the compounds according to the invention is, according to a further embodiment, NA²A² where A² and A² independently of one another are hydrogen, C₁-C₁₀-alkyl, C₁-C₁₀-haloalkyl, C₃-C₁₀-cycloalkyl, C₃-C₁₀-halocycloalkyl, C₁-C₁₀-alkoxy-C₁-C₁₀-alkyl, amino-C₁-C₁₀-alkyl; or independently of one another are phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy.

According to one embodiment A² and A² independently of one another are hydrogen. According to a further embodiment A² and A² independently of one another are hydrogen or C₁-C₁₀-alkyl, preferably methyl, ethyl, n-propyl, i-propyl, n-butyl, i-butyl, t-butyl. In a special embodiment of the invention, R² is NH₂, NHCH₃, N(CH₃)₂, NHC₂H₅, NHn-C₃H₇, NHi-C₃H₇, NHn-C₄H₉, NHi-C₄H₉, NHt-C₄H₉. According to a further embodiment A² and A² independently of one another are hydrogen or C₃-C₁₀-cycloalkyl, preferably cyclopropyl, cyclobutyl, cyclopenyl, cyclohexyl. In a special embodiment of the invention, R² is cyclopropylamino, cyclobutylamino, cyclopentylamino, cyclohexylamino. According to a further embodiment A² and A² independently of one another are C₁-C₁₀-alkoxy-C₁-C₁₀-alkyl, preferably C₁-C₄-alkoxy-C₁-C₄-alkyl. In a special embodiment of the invention, R² is methoxyethylamino, methoxypropylamino, methoxybutylamino, ethoxyethylamino, ethoxypropylamino or ethoxybutylamino. In a special embodiment of the invention, R² is NH₂. In a further special embodiment of the invention, R² is N(CH₃)₂. In a further special embodiment of the invention, R² is N(C₂H₅)₂.

According to one embodiment A² and A² independently of one another are hydrogen or phenyl. According to one embodiment A² and A² independently of one another are hydrogen or benzyl. According to a further embodiment A² and A² independently of one another are hydrogen or naphthyl. According to a further embodiment A² and A² independently of one another are hydrogen or a saturated partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO, SO₂ groups as ring members. In a special embodiment of the invention, R² is NH₂. In a further special embodiment of the invention, R² is N(phenyl)₂. In a further special embodiment of the invention, R² is N(benzyl)₂.

According to one embodiment phenyl, benzyl, naphthyl and the saturated, partially unsaturated or aromatic 5-, 6-, 7-, 8-, 9- or 10-membered heterocycle may carry one, two, three or four identical or different substituents selected from the group consisting of Cl, I, F, Br, preferably Cl, F. According to a further embodiment the above mentioned groups may carry one, two, three or four identical or different substituents selected from the group consisting of methyl, ethyl, propyl, butyl, preferably methyl. According to a further embodiment the above mentioned groups may carry one, two, three or four identical or different substituents selected from the group consisting of partially or fully halogenated methyl, ethyl, propyl, butyl, preferably partially or fully halogenated methyl, in particular CF₃. According to a further embodiment the above mentioned groups may carry one, two, three or four identical or different substituents selected from the group consisting of methoxy, ethoxy, propoxy, butoxy, preferably methoxy. According to a further embodiment the above mentioned groups may carry one, two, three or four identical or different substituents selected from the group consisting of partially or fully halogenated methoxy, ethoxy, propoxy, butoxy, preferably partially or fully halogenated methoxy, in particular OCF₃. In a special embodiment of the invention, A² and/or A² are phenyl. In a further special embodiment of the invention, A² and/or A² are phenyl substituted by Cl, F, Br, I, CH₃, OCH₃, CF₃ or OCF₃. In a further special embodiment of the invention, A² and/or A² are benzyl substituted by Cl, F, Br, I, CH₃, OCH₃, CF₃ or OCF₃. In a further special embodiment of the invention, A² and/or A² are naphthyl substituted by Cl, F, Br, I, CH₃, OCH₃, CF₃ or OCF₃.

R² in the compounds according to the invention is, according to a further embodiment, a radical of the formula —C(═O)A⁴, —OC(═O)A⁴, —NA⁴C(═O)A⁴, —N═OA⁴ where A⁴ is hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy.

According to one embodiment A⁴ is H or C₁-C₁₀-alkyl, preferably methyl, ethyl, n-propyl, i-propyl, n-butyl, i-butyl or t-butyl. According to a further embodiment A⁴ is C₁-C₁₀-haloalkyl; preferrably fully or partially halogenated methyl, ethyl, n-propyl, i-propyl, n-butyl, i-butyl or t-butyl, in particular fully or partially halogenated methyl. According to a further embodiment A⁴ is C₂-C₁₀-alkenyl, preferably ethylene. According to a further embodiment A⁴ is C₂-C₁₀-alkynyl, preferred ethynyl or 1-propynyl. According to a further embodiment A⁴ is C₃-C₁₀-cycloalkyl, preferrably cyclopropyl, cyclobutyl or cyclopentyl, in particular cyclopropyl or cyclobutyl. According to a further embodiment A⁴ is C₃-C₁₀-halocycloalkyl, preferrably fully or partially halogenated cyclopropyl, cyclobutyl or cyclopentyl, in particular fully or partially halogenated cyclopropyl. According to a further embodiment A⁴ is C₃-C₁₀-cycloalkenyl, preferably cyclopropenyl. According to a further embodiment A⁴ is NH₂. According to a further embodiment A⁴ is mono-(C₁-C₁₀-alkyl)amino. According to a further embodiment A⁴ is di-(C₁-C₁₀-alkyl)amino.

In a special embodiment of the invention, R² is —C(═O)CH₃. In a further special embodiment of the invention, R² is —C(═O)CH₂CH₃. In a further special embodiment of the invention, R² is —C(═O)CH(CH₃)₂. In a further special embodiment of the invention, R² is —C(═O)C(CH₃)₃. In a further special embodiment of the invention, R² is —C(═O)OH. In a further special embodiment of the invention, R² is —C(═O)OCH₃. In a further special embodiment of the invention, R² is —C(═O)OCH₂CH₃. In a further special embodiment of the invention, R² is —C(═O)OCH(CH₃)₂. In a further special embodiment of the invention, R² is —C(═O)OC(CH₃)₃. In a further special embodiment of the invention, R² is —C(═O)NH₂. In a further special embodiment of the invention, R² is —C(═O)NHCH₃. In a further special embodiment of the invention, R² is —C(═O)NHCH₂CH₃. In a further special embodiment of the invention, R² is —C(═O)NHCH(CH₃)₂. In a further special embodiment of the invention, R² is —C(═O)NHC(CH₃)₃. In a further special embodiment of the invention, R² is —C(═O)N(CH₃)₂. In a further special embodiment of the invention, R² is —C(═O)N(CH₂CH₃)₂. In a further special embodiment of the invention, R² is —C(═O)N(CH(CH₃)₂)₂. In a further special embodiment of the invention, R² is —C(═O)N(C(CH₃)₃)₂.

According to one embodiment A⁴ is phenyl. According to a further embodiment A⁴ is benzyl. According to a further embodiment A⁴ is naphthyl. According to one embodiment A⁴ is a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members.

According to one embodiment phenyl, benzyl, naphthyl and the saturated, partially unsaturated or aromatic 5-, 6-, 7-, 8-, 9- or 10-membered heterocycle may carry one, two, three or four identical or different substituents selected from the group consisting of Cl, I, F, Br, preferably Cl, F. According to a further embodiment the above mentioned groups may carry one, two, three or four identical or different substituents selected from the group consisting of methyl, ethyl, propyl, butyl, preferably methyl. According to a further embodiment the above mentioned groups may carry one, two, three or four identical or different substituents selected from the group consisting of partially or fully halogenated methyl, ethyl, propyl, butyl, preferably partially or fully halogenated methyl, in particular CF₃. According to a further embodiment the above mentioned groups may carry one, two, three or four identical or different substituents selected from the group consisting of methoxy, ethoxy, propoxy, butoxy, preferably methoxy. According to a further embodiment the above mentioned groups may carry one, two, three or four identical or different substituents selected from the group consisting of partially or fully halogenated methoxy, ethoxy, propoxy, butoxy, preferably partially or fully halogenated methoxy, in particular OCF₃.

R² in the compounds according to the invention is, according to a further embodiment, a radical of the formula —S(O)ₙA⁵, —OS(O)ₙA⁵, —NA⁵S(O)ₙA⁵ where n=0, 1, 2 and A⁵ independently of one another are hydrogen, hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated or unsaturated aromatic or non-aromatic 5-, 6-, 7-, 8, 9- or 10-membered heterocycle, where the heterocycle contains 1, 2, 3 or 4 heteroatoms selected from the group consisting of O, N and S as ring members and may furthermore contain one or two CO groups as ring members, wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy.

According to one embodiment A⁵ is H or C₁-C₁₀-alkyl, preferably methyl, ethyl, n-propyl, i-propyl, n-butyl, i-butyl or t-butyl. According to a further embodiment A⁵ is C₁-C₁₀-haloalkyl; preferrably fully or partially halogenated methyl, ethyl, n-propyl, i-propyl, n-butyl, i-butyl or t-butyl, in particular fully or partially halogenated methyl. According to a further embodiment A⁵ is C₂-C₁₀-alkenyl, preferably ethylene. According to a further embodiment A⁵ is C₂-C₁₀-alkynyl, preferred ethynyl or 1-propynyl. According to a further embodiment A⁵ is C₃-C₁₀-cycloalkyl, preferrably cyclopropyl, cyclobutyl or cyclopentyl, in particular cyclopropyl or cyclobutyl. According to a further embodiment A⁵ is C₃-C₁₀-halocycloalkyl, preferrably fully or partially halogenated cyclopropyl, cyclobutyl or cyclopentyl, in particular fully or partially halogenated cyclopropyl. According to a further embodiment A⁵ is C₃-C₁₀-cycloalkenyl, preferably cyclopropenyl. According to a further embodiment A⁵ is NH₂. According to a further embodiment A⁵ is mono-(C₁-C₁₀-alkyl)amino. According to a further embodiment A⁵ is di-(C₁-C₁₀-alkyl)amino.

According to one embodiment A⁵ is phenyl. According to a further embodiment A⁵ is benzyl. According to a further embodiment A⁵ is naphthyl. According to one embodiment A⁵ is a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members.

According to one embodiment phenyl, benzyl, naphthyl and the saturated, partially unsaturated or aromatic 5-, 6-, 7-, 8-, 9- or 10-membered heterocycle may carry one, two, three or four identical or different substituents selected from the group consisting of Cl, I, F, Br, preferably Cl, F. According to a further embodiment the above mentioned groups may carry one, two, three or four identical or different substituents selected from the group consisting of methyl, ethyl, propyl, butyl, preferably methyl. According to a further embodiment the above mentioned groups may carry one, two, three or four identical or different substituents selected from the group consisting of partially or fully halogenated methyl, ethyl, propyl, butyl, preferably partially or fully halogenated methyl, in particular CF₃. According to a further embodiment the above mentioned groups may carry one, two, three or four identical or different substituents selected from the group consisting of methoxy, ethoxy, propoxy, butoxy, preferably methoxy. According to a further embodiment the above mentioned groups may carry one, two, three or four identical or different substituents selected from the group consisting of partially or fully halogenated methoxy, ethoxy, propoxy, butoxy, preferably partially or fully halogenated methoxy, in particular OCF₃.

In a special embodiment of the invention, R² is —SCH₃. In a further special embodiment of the invention, R² is —S(O)₂CH₃.

According to one embodiment, the present invention relates to compounds of the formula I.A

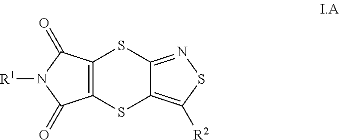

Here, the variables are as defined elsewhere herein for formula I, or as defined as being preferred for formula I.

According to one embodiment, the present invention relates to compounds of the formula I.B

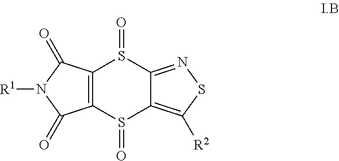

Here, the variables are as defined elsewhere herein for formula I, or as defined as being preferred for formula I.

According to one embodiment, the present invention relates to compounds of the formula I.C

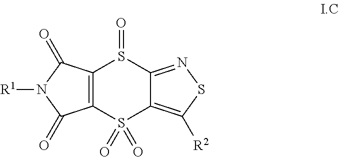

Here, the variables are as defined elsewhere herein for formula I, or as defined as being preferred for formula I.

According to one embodiment, the present invention relates to compounds of the formula I.D

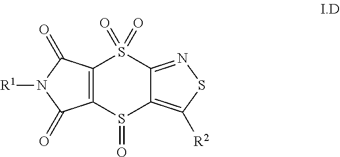

Here, the variables are as defined elsewhere herein for formula I, or as defined as being preferred for formula I.

According to one embodiment, the present invention relates to compounds of the formula I.E

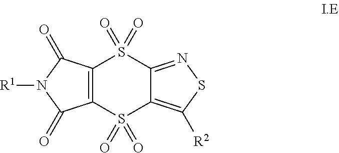

Here, the variables are as defined elsewhere herein for formula I, or as defined as being preferred for formula I.

Preference is given to the compounds I according to the invention compiled in Tables 1a to 42a, 1b to 42b, 1c to 42c, 1d to 42d, 1e to 42e below with the proviso as defined above. The groups mentioned for a substituent in the tables are furthermore per se, independently of the combination in which they are mentioned, a particularly preferred aspect of the substituent in question.

Table 1a

Compounds of the formula I.A in which R¹ corresponds to line A-1 of Table A and the meaning R² for each individual compound corresponds in each case to one line of Table B (compounds I.A.A1.B1 to I.A.A1.B21B21).

Table 2a

Compounds of the formula I.A in which R¹ corresponds to line A-2 of Table A and the meaning for R² for each individual compound corresponds in each case to one line of Table B (compounds I.A.A2.B1 to I.A.A2.B21B21).

Table 3a

Compounds of the formula I.A in which R¹ corresponds to line A-3 of Table A and the meaning for R² for each individual compound corresponds in each case to one line of Table B (compounds I.A.A3.B1 to I.A.A3.B21B21).

Table 4a

Compounds of the formula I.A in which R¹ corresponds to line A-4 of Table A and the meaning for R² for each individual compound corresponds in each case to one line of Table B (compounds I.A.A4.B1 to I.A.A4.B21B21).

Table 5a

Compounds of the formula I.A in which R¹ corresponds to line A-5 of Table A and the meaning for R² for each individual compound corresponds in each case to one line of Table B (compounds I.A.A5.B1 to I.A.A5.B21B21).

Table 6a

Compounds of the formula I.A in which R¹ corresponds to line A-6 of Table A and the meaning for R² for each individual compound corresponds in each case to one line of Table B (compounds I.A.A6.B1 to I.A.A6.B21B21).

Table 7a

Compounds of the formula I.A in which R¹ corresponds to line A-7 of Table A and the meaning for R² for each individual compound corresponds in each case to one line of Table B (compounds I.A.A7.B1 to I.A.A7.B21B21).

Table 8a

Compounds of the formula I.A in which R¹ corresponds to line A-8 of Table A and the meaning for R² for each individual compound corresponds in each case to one line of Table B (compounds I.A.A8.B1 to I.A.A8.B21B21).

Table 9a

Compounds of the formula I.A in which R¹ corresponds to line A-9 of Table A and the meaning for R² for each individual compound corresponds in each case to one line of Table B (compounds I.A.A9.B1 to I.A.A9.B21B21).

Table 10a

Compounds of the formula I.A in which R¹ corresponds to line A-10 of Table A and the meaning for R² for each individual compound corresponds in each case to one line of Table B (compounds I.A.A10.B1 to I.A.A10.B21B21).

Table 11a

Compounds of the formula I.A in which R¹ corresponds to line A-11 of Table A and the meaning for R² for each individual compound corresponds in each case to one line of Table B (compounds I.A.A11.B1 to I.A.A11.B21.B21).

Table 12a

Compounds of the formula I.A in which R¹ corresponds to line A-12 of Table A and the meaning for R² for each individual compound corresponds in each case to one line of Table B (compounds I.A.A12.B1 to I.A.A12.B21.B21).

Table 13a

Compounds of the formula I.A in which R¹ corresponds to line A-13 of Table A and the meaning for R² for each individual compound corresponds in each case to one line of Table B (compounds I.A.A13.B1 to I.A.A13.B21.B21).

Table 14a

Compounds of the formula I.A in which R¹ corresponds to line A-14 of Table A and the meaning for R² for each individual compound corresponds in each case to one line of Table B (compounds I.A.A14.B1 to I.A.A14.B21.B21).

Table 15a

Compounds of the formula I.A in which R¹ corresponds to line A-15 of Table A and the meaning for R² for each individual compound corresponds in each case to one line of Table B (compounds I.A.A15.B1 to I.A.A15.B21).

Table 16a

Compounds of the formula I.A in which R¹ corresponds to line A-16 of Table A and the meaning for R² for each individual compound corresponds in each case to one line of Table B (compounds I.A.A16.B1 to I.A.A16.B21).

Table 17a

Compounds of the formula I.A in which R¹ corresponds to line A-17 of Table A and the meaning for R² for each individual compound corresponds in each case to one line of Table B (compounds I.A.A17.B1 to I.A.A17.B21).

Table 18a

Compounds of the formula I.A in which R¹ corresponds to line A-18 of Table A and the meaning for R² for each individual compound corresponds in each case to one line of Table B (compounds I.A.A18.B1 to I.A.A18.B21).

Table 19a

Compounds of the formula I.A in which R¹ corresponds to line A-19 of Table A and the meaning for R² for each individual compound corresponds in each case to one line of Table B (compounds I.A.A19.B1 to I.A.A19.B21).

Table 20a

Compounds of the formula I.A in which R¹ corresponds to line A-20 of Table A and the meaning for R² for each individual compound corresponds in each case to one line of Table B (compounds I.A.A20.B1 to I.A.A20.B21).

Table 21a

Compounds of the formula I.A in which R¹ corresponds to line A-21 of Table A and the meaning for R² for each individual compound corresponds in each case to one line of Table B (compounds I.A.A21.B1 to I.A.A21.B21).

Table 22a

Compounds of the formula I.A in which R¹ corresponds to line A-22 of Table A and the meaning for the combination of (R⁴)ₘ and R³² for each individual compound corresponds in each case to one line of Table B (compounds I.A.A22.B1 to I.A.A22.B21).

Table 23a

Compounds of the formula I.A in which R¹ corresponds to line A-23 of Table A and the meaning for R² for each individual compound corresponds in each case to one line of Table B (compounds I.A.A23.B1 to I.A.A23.B21).

Table 24a

Compounds of the formula I.A in which R¹ corresponds to line A-24 of Table A and the meaning for R² for each individual compound corresponds in each case to one line of Table B (compounds I.A.A24.B1 to I.A.A24.B21).

Table 25a

Compounds of the formula I.A in which R¹ corresponds to line A-25 of Table A and the meaning for R² for each individual compound corresponds in each case to one line of Table B (compounds I.A.A25.B1 to I.A.A25.B21).

Table 26a

Compounds of the formula I.A in which R¹ corresponds to line A-26 of Table A and the meaning for R² for each individual compound corresponds in each case to one line of Table B (compounds I.A.A26.B1 to I.A.A26.B21).

Table 27a

Compounds of the formula I.A in which R¹ corresponds to line A-27 of Table A and the meaning for R² for each individual compound corresponds in each case to one line of Table B (compounds I.A.A27.B1 to I.A.A27.B21).

Table 28a

Compounds of the formula I.A in which R¹ corresponds to line A-28 of Table A and the meaning for R² for each individual compound corresponds in each case to one line of Table B (compounds I.A.A28.B1 to I.A.A28.B21).

Table 29a

Compounds of the formula I.A in which R¹ corresponds to line A-29 of Table A and the meaning for R² for each individual compound corresponds in each case to one line of Table B (compounds I.A.A29.B1 to I.A.A29.B21).

Table 30a

Compounds of the formula I.A in which R¹ corresponds to line A-30 of Table A and the meaning for R² for each individual compound corresponds in each case to one line of Table B (compounds I.A.A30.B1 to I.A.A30.B21).

Table 31a

Compounds of the formula I.A in which R¹ corresponds to line A-31 of Table A and the meaning for R² for each individual compound corresponds in each case to one line of Table B (compounds I.A.A31.B1 to I.A.A31.B21).

Table 32a

Compounds of the formula I.A in which R¹ corresponds to line A-32 of Table A and the meaning for R² for each individual compound corresponds in each case to one line of Table B (compounds I.A.A32.B1 to I.A.A32.B21).

Table 33a

Compounds of the formula I.A in which R¹ corresponds to line A-33 of Table A and the meaning for R² for each individual compound corresponds in each case to one line of Table B (compounds I.A.A33.B1 to I.A.A33.B21).

Table 34a

Compounds of the formula I.A in which R¹ corresponds to line A-34 of Table A and the meaning for R² for each individual compound corresponds in each case to one line of Table B (compounds I.A.A34.B1 to I.A.A34.B21).

Table 35a

Compounds of the formula I.A in which R¹ corresponds to line A-35 of Table A and the meaning for R² for each individual compound corresponds in each case to one line of Table B (compounds I.A.A35.B1 to I.A.A35.B21).

Table 36a

Compounds of the formula I.A in which R¹ corresponds to line A-36 of Table A and the meaning for R² for each individual compound corresponds in each case to one line of Table B (compounds I.A.A36.B1 to I.A.A36.B21).

Table 37a

Compounds of the formula I.A in which R¹ corresponds to line A-37 of Table A and the meaning for R² for each individual compound corresponds in each case to one line of Table B (compounds I.A.A37.B1 to I.A.A37.B21).

Table 38a

Compounds of the formula I.A in which R¹ corresponds to line A-38 of Table A and the meaning for R² for each individual compound corresponds in each case to one line of Table B (compounds I.A.A38.B1 to I.A.A38.B21).

Table 39a

Compounds of the formula I.A in which R¹ corresponds to line A-39 of Table A and the meaning for R² for each individual compound corresponds in each case to one line of Table B (compounds I.A.A39.B1 to I.A.A39.B21).

Table 40a

Compounds of the formula I.A in which R¹ corresponds to line A-40 of Table A and the meaning for R² for each individual compound corresponds in each case to one line of Table B (compounds I.A.A40.B1 to I.A.A40.B21).

Table 41a

Compounds of the formula I.A in which R¹ corresponds to line A-41 of Table A and the meaning for R² for each individual compound corresponds in each case to one line of Table B (compounds I.A.A41.B1 to I.A.A41.B21).

Table 42a

Compounds of the formula I.A in which R¹ corresponds to line A-42 of Table A and the meaning for R² for each individual compound corresponds in each case to one line of Table B (compounds I.A.A42.B1 to I.A.A42.B21).

Table 43a

Compounds of the formula I.A in which R¹ corresponds to line A-43 of Table A and the meaning for R² for each individual compound corresponds in each case to one line of Table B (compounds I.A.A43.B1 to I.A.A43.B21).

Table 44a

Compounds of the formula I.A in which R¹ corresponds to line A-44 of Table A and the meaning for R² for each individual compound corresponds in each case to one line of Table B (compounds I.A.A44.B1 to I.A.A44.B21).

Table 45a

Compounds of the formula I.A in which R¹ corresponds to line A-45 of Table A and the meaning for R² for each individual compound corresponds in each case to one line of Table B (compounds I.A.A45.B1 to I.A.A45.B21).

Table 46a

Compounds of the formula I.A in which R¹ corresponds to line A-46 of Table A and the meaning for R² for each individual compound corresponds in each case to one line of Table B (compounds I.A.A46.B1 to I.A.A46.B21).

Table 47a

Compounds of the formula I.A in which R¹ corresponds to line A-47 of Table A and the meaning for R² for each individual compound corresponds in each case to one line of Table B (compounds I.A.A47.B1 to I.A.A47.B21).

Table 48a

Compounds of the formula I.A in which R¹ corresponds to line A-48 of Table A and the meaning for R² for each individual compound corresponds in each case to one line of Table B (compounds I.A.A48.B1 to I.A.A48.B21).

Table 49a

Compounds of the formula I.A in which R¹ corresponds to line A-49 of Table A and the meaning for R² for each individual compound corresponds in each case to one line of Table B (compounds I.A.A49.B1 to I.A.A49.B21).

Table 50a

Compounds of the formula I.A in which R¹ corresponds to line A-50 of Table A and the meaning for R² for each individual compound corresponds in each case to one line of Table B (compounds I.A.A50.B1 to I.A.A50.B21).

Table 51a

Compounds of the formula I.A in which R¹ corresponds to line A-51 of Table A and the meaning for R² for each individual compound corresponds in each case to one line of Table B (compounds I.A.A51.B1 to I.A.A51.B21).

Table 52a

Compounds of the formula I.A in which R¹ corresponds to line A-52 of Table A and the meaning for R² for each individual compound corresponds in each case to one line of Table B (compounds I.A.A52.B1 to I.A.A52.B21).

Table 53a

Compounds of the formula I.A in which R¹ corresponds to line A-53 of Table A and the meaning for R² for each individual compound corresponds in each case to one line of Table B (compounds I.A.A53.B1 to I.A.A53.B21).

Table 54a

Compounds of the formula I.A in which R¹ corresponds to line A-54 of Table A and the meaning for R² for each individual compound corresponds in each case to one line of Table B (compounds I.A.A54.B1 to I.A.A54.B21).

Table 55a

Compounds of the formula I.A in which R¹ corresponds to line A-55 of Table A and the meaning for R² for each individual compound corresponds in each case to one line of Table B (compounds I.A.A55.B1 to I.A.A55.B21).

Table 56a

Compounds of the formula I.A in which R¹ corresponds to line A-56 of Table A and the meaning for R² for each individual compound corresponds in each case to one line of Table B (compounds I.A.A56.B1 to I.A.A56.B21).

Table 57a

Compounds of the formula I.A in which R¹ corresponds to line A-57 of Table A and the meaning for R² for each individual compound corresponds in each case to one line of Table B (compounds I.A.A57.B1 to I.A.A57.B21).

Table 58a

Compounds of the formula I.A in which R¹ corresponds to line A-58 of Table A and the meaning for R² for each individual compound corresponds in each case to one line of Table B (compounds I.A.A58.B1 to I.A.A58.B21).

Table 59a

Compounds of the formula I.A in which R¹ corresponds to line A-59 of Table A and the meaning for R² for each individual compound corresponds in each case to one line of Table B (compounds I.A.A59.B1 to I.A.A59.B21).

Table 60a

Compounds of the formula I.A in which R¹ corresponds to line A-60 of Table A and the meaning for R² for each individual compound corresponds in each case to one line of Table B (compounds I.A.A60.B1 to I.A.A60.B21).

Table 61a

Compounds of the formula I.A in which R¹ corresponds to line A-61 of Table A and the meaning for R² for each individual compound corresponds in each case to one line of Table B (compounds I.A.A61.B1 to I.A.A61.B21).

Table 62a

Compounds of the formula I.A in which R¹ corresponds to line A-62 of Table A and the meaning for R² for each individual compound corresponds in each case to one line of Table B (compounds I.A.A62.B1 to I.A.A62.B21).

Table 63a

Compounds of the formula I.A in which R¹ corresponds to line A-63 of Table A and the meaning for R² for each individual compound corresponds in each case to one line of Table B (compounds I.A.A63.B1 to I.A.A63.B21).

Table 64a

Compounds of the formula I.A in which R¹ corresponds to line A-64 of Table A and the meaning for R² for each individual compound corresponds in each case to one line of Table B (compounds I.A.A64.B1 to I.A.A64.B21).

Table 65a

Compounds of the formula I.A in which R¹ corresponds to line A-65 of Table A and the meaning for R² for each individual compound corresponds in each case to one line of Table B (compounds I.A.A65.B1 to I.A.A65.B21).

Table 66a

Compounds of the formula I.A in which R¹ corresponds to line A-66 of Table A and the meaning for R² for each individual compound corresponds in each case to one line of Table B (compounds I.A.A66.B1 to I.A.A66.B21).

Table 67a

Compounds of the formula I.A in which R¹ corresponds to line A-67 of Table A and the meaning for R² for each individual compound corresponds in each case to one line of Table B (compounds I.A.A67.B1 to I.A.A67.B21).

Table 68a

Compounds of the formula I.A in which R¹ corresponds to line A-68 of Table A and the meaning for R² for each individual compound corresponds in each case to one line of Table B (compounds I.A.A68.B1 to I.A.A68.B21).

Table 69a

Compounds of the formula I.A in which R¹ corresponds to line A-69 of Table A and the meaning for R² for each individual compound corresponds in each case to one line of Table B (compounds I.A.A69.B1 to I.A.A69.B21).

Table 70a

Compounds of the formula I.A in which R¹ corresponds to line A-70 of Table A and the meaning for R² for each individual compound corresponds in each case to one line of Table B (compounds I.A.A70.B1 to I.A.A70.B21).

Table 71a

Compounds of the formula I.A in which R¹ corresponds to line A-71 of Table A and the meaning for R² for each individual compound corresponds in each case to one line of Table B (compounds I.A.A71.B1 to I.A.A71.B21).

Table 72a

Compounds of the formula I.A in which R¹ corresponds to line A-72 of Table A and the meaning for R² for each individual compound corresponds in each case to one line of Table B (compounds I.A.A72.B1 to I.A.A72.B21).

Table 73a

Compounds of the formula I.A in which R¹ corresponds to line A-73 of Table A and the meaning for R² for each individual compound corresponds in each case to one line of Table B (compounds I.A.A73.B1 to I.A.A73.B21).

Table 74a

Compounds of the formula I.A in which R¹ corresponds to line A-74 of Table A and the meaning for R² for each individual compound corresponds in each case to one line of Table B (compounds I.A.A74.B1 to I.A.A74B21).

Table 75a

Compounds of the formula I.A in which R¹ corresponds to line A-75 of Table A and the meaning for R² for each individual compound corresponds in each case to one line of Table B (compounds I.A.A75.B1 to I.A.A75.B21).

Table 76a

Compounds of the formula I.A in which R¹ corresponds to line A-76 of Table A and the meaning for R² for each individual compound corresponds in each case to one line of Table B (compounds I.A.A76.B1 to I.A.A76.B21).

Table 77a

Compounds of the formula I.A in which R¹ corresponds to line A-77 of Table A and the meaning for R² for each individual compound corresponds in each case to one line of Table B (compounds I.A.A77.B1 to I.A.A77.B21).

Table 78a

Compounds of the formula I.A in which R¹ corresponds to line A-78 of Table A and the meaning for R² for each individual compound corresponds in each case to one line of Table B (compounds I.A.A78.B1 to I.A.A78.B21).

Table 79a

Compounds of the formula I.A in which R¹ corresponds to line A-79 of Table A and the meaning for R² for each individual compound corresponds in each case to one line of Table B (compounds I.A.A79.B1 to I.A.A79.B21).

Table 80a

Compounds of the formula I.A in which R¹ corresponds to line A-80 of Table A and the meaning for R² for each individual compound corresponds in each case to one line of Table B (compounds I.A.A80.B1 to I.A.A80.B21).

Table 81a

Compounds of the formula I.A in which R¹ corresponds to line A-81 of Table A and the meaning for R² for each individual compound corresponds in each case to one line of Table B (compounds I.A.A81.B1 to I.A.A81.B21).

Table 82a

Compounds of the formula I.A in which R¹ corresponds to line A-82 of Table A and the meaning for R² for each individual compound corresponds in each case to one line of Table B (compounds I.A.A82.B1 to I.A.A82.B21).

Table 83a

Compounds of the formula I.A in which R¹ corresponds to line A-83 of Table A and the meaning for R² for each individual compound corresponds in each case to one line of Table B (compounds I.A.A83.B1 to I.A.A83.B21).

Table 84a

Compounds of the formula I.A in which R¹ corresponds to line A-84 of Table A and the meaning for R² for each individual compound corresponds in each case to one line of Table B (compounds I.A.A84.B1 to I.A.A84.B21).

Table 85a

Compounds of the formula I.A in which R¹ corresponds to line A-85 of Table A and the meaning for R² for each individual compound corresponds in each case to one line of Table B (compounds I.A.A85.B1 to I.A.A85.B21).

Table 86a

Compounds of the formula I.A in which R¹ corresponds to line A-86 of Table A and the meaning for R² for each individual compound corresponds in each case to one line of Table B (compounds I.A.A86.B1 to I.A.A86.B21).

Table 87a

Compounds of the formula I.A in which R¹ corresponds to line A-87 of Table A and the meaning for R² for each individual compound corresponds in each case to one line of Table B (compounds I.A.A87.B1 to I.A.A87.B21).

Table 88a

Compounds of the formula I.A in which R¹ corresponds to line A-88 of Table A and the meaning for R² for each individual compound corresponds in each case to one line of Table B (compounds I.A.A88.B1 to I.A.A88.B21).

Table 89a

Compounds of the formula I.A in which R¹ corresponds to line A-89 of Table A and the meaning for R² for each individual compound corresponds in each case to one line of Table B (compounds I.A.A89.B1 to I.A.A89.B21).

Table 90a

Compounds of the formula I.A in which R¹ corresponds to line A-90 of Table A and the meaning for R² for each individual compound corresponds in each case to one line of Table B (compounds I.A.A90.B1 to I.A.A90.B21).

Table 91a

Compounds of the formula I.A in which R¹ corresponds to line A-91 of Table A and the meaning for R² for each individual compound corresponds in each case to one line of Table B (compounds I.A.A91.B1 to I.A.A91.B21).

Table 92a

Compounds of the formula I.A in which R¹ corresponds to line A-92 of Table A and the meaning for R² for each individual compound corresponds in each case to one line of Table B (compounds I.A.A92.B1 to I.A.A92.B21).

Table 93a

Compounds of the formula I.A in which R¹ corresponds to line A-93 of Table A and the meaning for R² for each individual compound corresponds in each case to one line of Table B (compounds I.A.A93.B1 to I.A.A93.B21).

Table 94a

Compounds of the formula I.A in which R¹ corresponds to line A-94 of Table A and the meaning for R² for each individual compound corresponds in each case to one line of Table B (compounds I.A.A94.B1 to I.A.A94.B21).

Table 95a

Compounds of the formula I.A in which R¹ corresponds to line A-95 of Table A and the meaning for R² for each individual compound corresponds in each case to one line of Table B (compounds I.A.A95.B1 to I.A.A95.B21).

Table 96a

Compounds of the formula I.A in which R¹ corresponds to line A-96 of Table A and the meaning for R² for each individual compound corresponds in each case to one line of Table B (compounds I.A.A96.B1 to I.A.A96.B21).

Table 97a

Compounds of the formula I.A in which R¹ corresponds to line A-97 of Table A and the meaning for R² for each individual compound corresponds in each case to one line of Table B (compounds I.A.A97.B1 to I.A.A97.B21).

Table 98a

Compounds of the formula I.A in which R¹ corresponds to line A-98 of Table A and the meaning for R² for each individual compound corresponds in each case to one line of Table B (compounds I.A.A98.B1 to I.A.A98.B21).

Table 99a

Compounds of the formula I.A in which R¹ corresponds to line A-99 of Table A and the meaning for R² for each individual compound corresponds in each case to one line of Table B (compounds I.A.A99.B1 to I.A.A99.B21).

Table 100a

Compounds of the formula I.A in which R¹ corresponds to line A-100 of Table A and the meaning for R² for each individual compound corresponds in each case to one line of Table B (compounds I.A.A100.B1 to I.A.A100.B21).

Table 101a

Compounds of the formula I.A in which R¹ corresponds to line A-101 of Table A and the meaning for R² for each individual compound corresponds in each case to one line of Table B (compounds I.A.A101.B1 to I.A.A101.B21).

Table 1b

Compounds of the formula I.B in which R¹ corresponds to line A-1 of Table A and the meaning R² for each individual compound corresponds in each case to one line of Table B (compounds I.B.A1.B1 to I.B.A1.B21).

Table 2b

Compounds of the formula I.B in which R¹ corresponds to line A-2 of Table A and the meaning for R² for each individual compound corresponds in each case to one line of Table B (compounds I.B.A2.B1 to I.B.A2.B21).

Table 3b

Compounds of the formula I.B in which R¹ corresponds to line A-3 of Table A and the meaning for R² for each individual compound corresponds in each case to one line of Table B (compounds I.B.A3.B1 to I.B.A3.B21).

Table 4b

Compounds of the formula I.B in which R¹ corresponds to line A-4 of Table A and the meaning for R² for each individual compound corresponds in each case to one line of Table B (compounds I.B.A4.B1 to I.B.A4.B21).

Table 5b

Compounds of the formula I.B in which R¹ corresponds to line A-5 of Table A and the meaning for R² for each individual compound corresponds in each case to one line of Table B (compounds I.B.A5.B1 to I.B.A5.B21).

Table 6b

Compounds of the formula I.B in which R¹ corresponds to line A-6 of Table A and the meaning for R² for each individual compound corresponds in each case to one line of Table B (compounds I.B.A6.B1 to I.B.A6.B21).

Table 7b

Compounds of the formula I.B in which R¹ corresponds to line A-7 of Table A and the meaning for R² for each individual compound corresponds in each case to one line of Table B (compounds I.B.A7.B1 to I.B.A7.B21).

Table 8b

Compounds of the formula I.B in which R¹ corresponds to line A-8 of Table A and the meaning for R² for each individual compound corresponds in each case to one line of Table B (compounds I.B.A8.B1 to I.B.A8.B21).

Table 9b

Compounds of the formula I.B in which R¹ corresponds to line A-9 of Table A and the meaning for R² for each individual compound corresponds in each case to one line of Table B (compounds I.B.A9.B1 to I.B.A9.B21).

Table 10b

Compounds of the formula I.B in which R¹ corresponds to line A-10 of Table A and the meaning for R² for each individual compound corresponds in each case to one line of Table B (compounds I.B.A10.B1 to I.B.A10.B21).

Table 11 b

Compounds of the formula I.B in which R¹ corresponds to line A-11 of Table A and the meaning for R² for each individual compound corresponds in each case to one line of Table B (compounds I.B.A11.B1 to I.B.A11.B21).

Table 12b

Compounds of the formula I.B in which R¹ corresponds to line A-12 of Table A and the meaning for R² for each individual compound corresponds in each case to one line of Table B (compounds I.B.A12.B1 to I.B.A12.B21).

Table 13b

Compounds of the formula I.B in which R¹ corresponds to line A-13 of Table A and the meaning for R² for each individual compound corresponds in each case to one line of Table B (compounds I.B.A13.B1 to I.B.A13.B21).

Table 14b

Compounds of the formula I.B in which R¹ corresponds to line A-14 of Table A and the meaning for R² for each individual compound corresponds in each case to one line of Table B (compounds I.B.A14.B1 to I.B.A14.B21).

Table 15b

Compounds of the formula I.B in which R¹ corresponds to line A-15 of Table A and the meaning for R² for each individual compound corresponds in each case to one line of Table B (compounds I.B.A15.B1 to I.B.A15.B21).

Table 16b

Compounds of the formula I.B in which R¹ corresponds to line A-16 of Table A and the meaning for R² for each individual compound corresponds in each case to one line of Table B (compounds I.B.A16.B1 to I.B.A16.B21).

Table 17b

Compounds of the formula I.B in which R¹ corresponds to line A-17 of Table A and the meaning for R² for each individual compound corresponds in each case to one line of Table B (compounds I.B.A17.B1 to I.B.A17.B21).

Table 18b

Compounds of the formula I.B in which R¹ corresponds to line A-18 of Table A and the meaning for R² for each individual compound corresponds in each case to one line of Table B (compounds I.B.A18.B1 to I.B.A18.B21).

Table 19b

Compounds of the formula I.B in which R¹ corresponds to line A-19 of Table A and the meaning for R² for each individual compound corresponds in each case to one line of Table B (compounds I.B.A19.B1 to I.B.A19.B21).

Table 20b

Compounds of the formula I.B in which R¹ corresponds to line A-20 of Table A and the meaning for R² for each individual compound corresponds in each case to one line of Table B (compounds I.B.A20.B1 to I.B.A20.B21).

Table 21b

Compounds of the formula I.B in which R¹ corresponds to line A-21 of Table A and the meaning for R² for each individual compound corresponds in each case to one line of Table B (compounds I.B.A21.B1 to I.B.A21.B21).

Table 22b

Compounds of the formula I.B in which R¹ corresponds to line A-22 of Table A and the meaning for the combination of (R⁴)ₘ and R³² for each individual compound corresponds in each case to one line of Table B (compounds I.B.A22.B1 to I.B.A22.B21).

Table 23b

Compounds of the formula I.B in which R¹ corresponds to line A-23 of Table A and the meaning for R² for each individual compound corresponds in each case to one line of Table B (compounds I.B.A23.B1 to I.B.A23.B21).

Table 24b

Compounds of the formula I.B in which R¹ corresponds to line A-24 of Table A and the meaning for R² for each individual compound corresponds in each case to one line of Table B (compounds I.B.A24.B1 to I.B.A24.B21).

Table 25b

Compounds of the formula I.B in which R¹ corresponds to line A-25 of Table A and the meaning for R² for each individual compound corresponds in each case to one line of Table B (compounds I.B.A25.B1 to I.B.A25.B21).

Table 26b

Compounds of the formula I.B in which R¹ corresponds to line A-26 of Table A and the meaning for R² for each individual compound corresponds in each case to one line of Table B (compounds I.B.A26.B1 to I.B.A26.B21).

Table 27b

Compounds of the formula I.B in which R¹ corresponds to line A-27 of Table A and the meaning for R² for each individual compound corresponds in each case to one line of Table B (compounds I.B.A27.B1 to I.B.A27.B21).

Table 28b

Compounds of the formula I.B in which R¹ corresponds to line A-28 of Table A and the meaning for R² for each individual compound corresponds in each case to one line of Table B (compounds I.B.A28.B1 to I.B.A28.B21).

Table 29b

Compounds of the formula I.B in which R¹ corresponds to line A-29 of Table A and the meaning for R² for each individual compound corresponds in each case to one line of Table B (compounds I.B.A29.B1 to I.B.A29.B21).

Table 30b

Compounds of the formula I.B in which R¹ corresponds to line A-30 of Table A and the meaning for R² for each individual compound corresponds in each case to one line of Table B (compounds I.B.A30.B1 to I.B.A30.B21).

Table 31b

Compounds of the formula I.B in which R¹ corresponds to line A-31 of Table A and the meaning for R² for each individual compound corresponds in each case to one line of Table B (compounds I.B.A31.B1 to I.B.A31.B21).

Table 32b

Compounds of the formula I.B in which R¹ corresponds to line A-32 of Table A and the meaning for R² for each individual compound corresponds in each case to one line of Table B (compounds I.B.A32.B1 to I.B.A32.B21).

Table 33b

Compounds of the formula I.B in which R¹ corresponds to line A-33 of Table A and the meaning for R² for each individual compound corresponds in each case to one line of Table B (compounds I.B.A33.B1 to I.B.A33.B21).

Table 34b

Compounds of the formula I.B in which R¹ corresponds to line A-34 of Table A and the meaning for R² for each individual compound corresponds in each case to one line of Table B (compounds I.B.A34.B1 to I.B.A34.B21).

Table 35b

Compounds of the formula I.B in which R¹ corresponds to line A-35 of Table A and the meaning for R² for each individual compound corresponds in each case to one line of Table B (compounds I.B.A35.B1 to I.B.A35.B21).

Table 36b

Compounds of the formula I.B in which R¹ corresponds to line A-36 of Table A and the meaning for R² for each individual compound corresponds in each case to one line of Table B (compounds I.B.A36.B1 to I.B.A36.B21).

Table 37b

Compounds of the formula I.B in which R¹ corresponds to line A-37 of Table A and the meaning for R² for each individual compound corresponds in each case to one line of Table B (compounds I.B.A37.B1 to I.B.A37.B21).

Table 38b

Compounds of the formula I.B in which R¹ corresponds to line A-38 of Table A and the meaning for R² for each individual compound corresponds in each case to one line of Table B (compounds I.B.A38.B1 to I.B.A38.B21).

Table 39b

Compounds of the formula I.B in which R¹ corresponds to line A-39 of Table A and the meaning for R² for each individual compound corresponds in each case to one line of Table B (compounds I.B.A39.B1 to I.B.A39.B21).

Table 40b

Compounds of the formula I.B in which R¹ corresponds to line A-40 of Table A and the meaning for R² for each individual compound corresponds in each case to one line of Table B (compounds I.B.A40.B1 to I.B.A40.B21).

Table 41b

Compounds of the formula I.B in which R¹ corresponds to line A-41 of Table A and the meaning for R² for each individual compound corresponds in each case to one line of Table B (compounds I.B.A41.B1 to I.B.A41.B21).

Table 42b

Compounds of the formula I.B in which R¹ corresponds to line A-42 of Table A and the meaning for R² for each individual compound corresponds in each case to one line of Table B (compounds I.B.A42.B1 to I.B.A42.B21).

Table 43b

Compounds of the formula I.B in which R¹ corresponds to line A-43 of Table A and the meaning for R² for each individual compound corresponds in each case to one line of Table B (compounds I.B.A43.B1 to I.B.A43.B21).

Table 44b

Compounds of the formula I.B in which R¹ corresponds to line A-44 of Table A and the meaning for R² for each individual compound corresponds in each case to one line of Table B (compounds I.B.A44.B1 to I.B.A44.B21).

Table 45b

Compounds of the formula I.B in which R¹ corresponds to line A-45 of Table A and the meaning for R² for each individual compound corresponds in each case to one line of Table B (compounds I.B.A45.B1 to I.B.A45.B21).

Table 46b

Compounds of the formula I.B in which R¹ corresponds to line A-46 of Table A and the meaning for R² for each individual compound corresponds in each case to one line of Table B (compounds I.B.A46.B1 to I.B.A46.B21).

Table 47b

Compounds of the formula I.B in which R¹ corresponds to line A-47 of Table A and the meaning for R² for each individual compound corresponds in each case to one line of Table B (compounds I.B.A47.B1 to I.B.A47.B21).

Table 48b

Compounds of the formula I.B in which R¹ corresponds to line A-48 of Table A and the meaning for R² for each individual compound corresponds in each case to one line of Table B (compounds I.B.A48.B1 to I.B.A48.B21).

Table 49b

Compounds of the formula I.B in which R¹ corresponds to line A-49 of Table A and the meaning for R² for each individual compound corresponds in each case to one line of Table B (compounds I.B.A49.B1 to I.B.A49.B21).

Table 50b

Compounds of the formula I.B in which R¹ corresponds to line A-50 of Table A and the meaning for R² for each individual compound corresponds in each case to one line of Table B (compounds I.B.A50.B1 to I.B.A50.B21).

Table 51b

Compounds of the formula I.B in which R¹ corresponds to line A-51 of Table A and the meaning for R² for each individual compound corresponds in each case to one line of Table B (compounds I.B.A51.B1 to I.B.A51.B21).

Table 52b

Compounds of the formula I.B in which R¹ corresponds to line A-52 of Table A and the meaning for R² for each individual compound corresponds in each case to one line of Table B (compounds I.B.A52.B1 to I.B.A52.B21).

Table 53b

Compounds of the formula I.B in which R¹ corresponds to line A-53 of Table A and the meaning for R² for each individual compound corresponds in each case to one line of Table B (compounds I.B.A53.B1 to I.B.A53.B21).

Table 54b

Compounds of the formula I.B in which R¹ corresponds to line A-54 of Table A and the meaning for R² for each individual compound corresponds in each case to one line of Table B (compounds I.B.A54.B1 to I.B.A54.B21).

Table 55b

Compounds of the formula I.B in which R¹ corresponds to line A-55 of Table A and the meaning for R² for each individual compound corresponds in each case to one line of Table B (compounds I.B.A55.B1 to I.B.A55.B21).

Table 56b

Compounds of the formula I.B in which R¹ corresponds to line A-56 of Table A and the meaning for R² for each individual compound corresponds in each case to one line of Table B (compounds I.B.A56.B1 to I.B.A56.B21).

Table 57b

Compounds of the formula I.B in which R¹ corresponds to line A-57 of Table A and the meaning for R² for each individual compound corresponds in each case to one line of Table B (compounds I.B.A57.B1 to I.B.A57.B21).

Table 58b

Compounds of the formula I.B in which R¹ corresponds to line A-58 of Table A and the meaning for R² for each individual compound corresponds in each case to one line of Table B (compounds I.B.A58.B1 to I.B.A58.B21).

Table 59b

Compounds of the formula I.B in which R¹ corresponds to line A-59 of Table A and the meaning for R² for each individual compound corresponds in each case to one line of Table B (compounds I.B.A59.B1 to I.B.A59.B21).

Table 60b

Compounds of the formula I.B in which R¹ corresponds to line A-60 of Table A and the meaning for R² for each individual compound corresponds in each case to one line of Table B (compounds I.B.A60.B1 to I.B.A60.B21).

Table 61b

Compounds of the formula I.B in which R¹ corresponds to line A-61 of Table A and the meaning for R² for each individual compound corresponds in each case to one line of Table B (compounds I.B.A61.B1 to I.B.A61.B21).

Table 62b

Compounds of the formula I.B in which R¹ corresponds to line A-62 of Table A and the meaning for R² for each individual compound corresponds in each case to one line of Table B (compounds I.B.A62.B1 to I.B.A62.B21).

Table 63b

Compounds of the formula I.B in which R¹ corresponds to line A-63 of Table A and the meaning for R² for each individual compound corresponds in each case to one line of Table B (compounds I.B.A63.B1 to I.B.A63.B21).

Table 64b

Compounds of the formula I.B in which R¹ corresponds to line A-64 of Table A and the meaning for R² for each individual compound corresponds in each case to one line of Table B (compounds I.B.A64.B1 to I.B.A64.B21).

Table 65b

Compounds of the formula I.B in which R¹ corresponds to line A-65 of Table A and the meaning for R² for each individual compound corresponds in each case to one line of Table B (compounds I.B.A65.B1 to I.B.A65.B21).

Table 66b

Compounds of the formula I.B in which R¹ corresponds to line A-66 of Table A and the meaning for R² for each individual compound corresponds in each case to one line of Table B (compounds I.B.A66.B1 to I.B.A66.B21).

Table 67b

Compounds of the formula I.B in which R¹ corresponds to line A-67 of Table A and the meaning for R² for each individual compound corresponds in each case to one line of Table B (compounds I.B.A67.B1 to I.B.A67.B21).

Table 68b

Compounds of the formula I.B in which R¹ corresponds to line A-68 of Table A and the meaning for R² for each individual compound corresponds in each case to one line of Table B (compounds I.B.A68.B1 to I.B.A68.B21).

Table 69b

Compounds of the formula I.B in which R¹ corresponds to line A-69 of Table A and the meaning for R² for each individual compound corresponds in each case to one line of Table B (compounds I.B.A69.B1 to I.B.A69.B21).

Table 70b

Compounds of the formula I.B in which R¹ corresponds to line A-70 of Table A and the meaning for R² for each individual compound corresponds in each case to one line of Table B (compounds I.B.A70.B1 to I.B.A70.B21).

Table 71b

Compounds of the formula I.B in which R¹ corresponds to line A-71 of Table A and the meaning for R² for each individual compound corresponds in each case to one line of Table B (compounds I.B.A71.B1 to I.B.A71.B21).

Table 72b

Compounds of the formula I.B in which R¹ corresponds to line A-72 of Table A and the meaning for R² for each individual compound corresponds in each case to one line of Table B (compounds I.B.A72.B1 to I.B.A72.B21).

Table 73b

Compounds of the formula I.B in which R¹ corresponds to line A-73 of Table A and the meaning for R² for each individual compound corresponds in each case to one line of Table B (compounds I.B.A73.B1 to I.B.A73.B21).

Table 74b

Compounds of the formula I.B in which R¹ corresponds to line A-74 of Table A and the meaning for R² for each individual compound corresponds in each case to one line of Table B (compounds I.B.A74.B1 to I.B.A74B21).

Table 75b

Compounds of the formula I.B in which R¹ corresponds to line A-75 of Table A and the meaning for R² for each individual compound corresponds in each case to one line of Table B (compounds I.B.A75.B1 to I.B.A75.B21).

Table 76b

Compounds of the formula I.B in which R¹ corresponds to line A-76 of Table A and the meaning for R² for each individual compound corresponds in each case to one line of Table B (compounds I.B.A76.B1 to I.B.A76.B21).

Table 77b

Compounds of the formula I.B in which R¹ corresponds to line A-77 of Table A and the meaning for R² for each individual compound corresponds in each case to one line of Table B (compounds I.B.A77.B1 to I.B.A77.B21).

Table 78b

Compounds of the formula I.B in which R¹ corresponds to line A-78 of Table A and the meaning for R² for each individual compound corresponds in each case to one line of Table B (compounds I.B.A78.B1 to I.B.A78.B21).

Table 79b

Compounds of the formula I.B in which R¹ corresponds to line A-79 of Table A and the meaning for R² for each individual compound corresponds in each case to one line of Table B (compounds I.B.A79.B1 to I.B.A79.B21).

Table 80b

Compounds of the formula I.B in which R¹ corresponds to line A-80 of Table A and the meaning for R² for each individual compound corresponds in each case to one line of Table B (compounds I.B.A80.B1 to I.B.A80.B21).

Table 81b

Compounds of the formula I.B in which R¹ corresponds to line A-81 of Table A and the meaning for R² for each individual compound corresponds in each case to one line of Table B (compounds I.B.A81.B1 to I.B.A81.B21).

Table 82b

Compounds of the formula I.B in which R¹ corresponds to line A-82 of Table A and the meaning for R² for each individual compound corresponds in each case to one line of Table B (compounds I.B.A82.B1 to I.B.A82.B21).

Table 83b

Compounds of the formula I.B in which R¹ corresponds to line A-83 of Table A and the meaning for R² for each individual compound corresponds in each case to one line of Table B (compounds I.B.A83.B1 to I.B.A83.B21).

Table 84b

Compounds of the formula I.B in which R¹ corresponds to line A-84 of Table A and the meaning for R² for each individual compound corresponds in each case to one line of Table B (compounds I.B.A84.B1 to I.B.A84.B21).

Table 85b

Compounds of the formula I.B in which R¹ corresponds to line A-85 of Table A and the meaning for R² for each individual compound corresponds in each case to one line of Table B (compounds I.B.A85.B1 to I.B.A85.B21).

Table 86b

Compounds of the formula I.B in which R¹ corresponds to line A-86 of Table A and the meaning for R² for each individual compound corresponds in each case to one line of Table B (compounds I.B.A86.B1 to I.B.A86.B21).

Table 87b

Compounds of the formula I.B in which R¹ corresponds to line A-87 of Table A and the meaning for R² for each individual compound corresponds in each case to one line of Table B (compounds I.B.A87.B1 to I.B.A87.B21).

Table 88b

Compounds of the formula I.B in which R¹ corresponds to line A-88 of Table A and the meaning for R² for each individual compound corresponds in each case to one line of Table B (compounds I.B.A88.B1 to I.B.A88.B21).

Table 89b

Compounds of the formula I.B in which R¹ corresponds to line A-89 of Table A and the meaning for R² for each individual compound corresponds in each case to one line of Table B (compounds I.B.A89.B1 to I.B.A89.B21).

Table 90b

Compounds of the formula I.B in which R¹ corresponds to line A-90 of Table A and the meaning for R² for each individual compound corresponds in each case to one line of Table B (compounds I.B.A90.B1 to I.B.A90.B21).

Table 91b

Compounds of the formula I.B in which R¹ corresponds to line A-91 of Table A and the meaning for R² for each individual compound corresponds in each case to one line of Table B (compounds I.B.A91.B1 to I.B.A91.B21).

Table 92b

Compounds of the formula I.B in which R¹ corresponds to line A-92 of Table A and the meaning for R² for each individual compound corresponds in each case to one line of Table B (compounds I.B.A92.B1 to I.B.A92.B21).

Table 93b

Compounds of the formula I.B in which R¹ corresponds to line A-93 of Table A and the meaning for R² for each individual compound corresponds in each case to one line of Table B (compounds I.B.A93.B1 to I.B.A93.B21).

Table 94b

Compounds of the formula I.B in which R¹ corresponds to line A-94 of Table A and the meaning for R² for each individual compound corresponds in each case to one line of Table B (compounds I.B.A94.B1 to I.B.A94.B21).

Table 95b

Compounds of the formula I.B in which R¹ corresponds to line A-95 of Table A and the meaning for R² for each individual compound corresponds in each case to one line of Table B (compounds I.B.A95.B1 to I.B.A95.B21).

Table 96b

Compounds of the formula I.B in which R¹ corresponds to line A-96 of Table A and the meaning for R² for each individual compound corresponds in each case to one line of Table B (compounds I.B.A96.B1 to I.B.A96.B21).

Table 97b

Compounds of the formula I.B in which R¹ corresponds to line A-97 of Table A and the meaning for R² for each individual compound corresponds in each case to one line of Table B (compounds I.B.A97.B1 to I.B.A97.B21).

Table 98b

Compounds of the formula I.B in which R¹ corresponds to line A-98 of Table A and the meaning for R² for each individual compound corresponds in each case to one line of Table B (compounds I.B.A98.B1 to I.B.A98.B21).

Table 99b

Compounds of the formula I.B in which R¹ corresponds to line A-99 of Table A and the meaning for R² for each individual compound corresponds in each case to one line of Table B (compounds I.B.A99.B1 to I.B.A99.B21).

Table 100b

Compounds of the formula I.B in which R¹ corresponds to line A-100 of Table A and the meaning for R² for each individual compound corresponds in each case to one line of Table B (compounds I.B.A100.B1 to I.B.A100.B21).

Table 101b

Compounds of the formula I.B in which R¹ corresponds to line A-101 of Table A and the meaning for R² for each individual compound corresponds in each case to one line of Table B (compounds I.B.A101.B1 to I.B.A101.B21).

Table 102b

Table 1c

Compounds of the formula I.C in which R¹ corresponds to line A-1 of Table A and the meaning R² for each individual compound corresponds in each case to one line of Table B (compounds I.C.A1.B1 to I.C.A1.B21).

Table 2c

Compounds of the formula I.C in which R¹ corresponds to line A-2 of Table A and the meaning for R² for each individual compound corresponds in each case to one line of Table B (compounds I.C.A2.B1 to I.C.A2.B21).

Table 3c

Compounds of the formula I.C in which R¹ corresponds to line A-3 of Table A and the meaning for R² for each individual compound corresponds in each case to one line of Table B (compounds I.C.A3.B1 to I.C.A3.B21).

Table 4c

Compounds of the formula I.C in which R¹ corresponds to line A-4 of Table A and the meaning for R² for each individual compound corresponds in each case to one line of Table B (compounds I.C.A4.B1 to I.C.A4.B21).

Table 5c

Compounds of the formula I.C in which R¹ corresponds to line A-5 of Table A and the meaning for R² for each individual compound corresponds in each case to one line of Table B (compounds I.C.A5.B1 to I.C.A5.B21).

Table 6c

Compounds of the formula I.C in which R¹ corresponds to line A-6 of Table A and the meaning for R² for each individual compound corresponds in each case to one line of Table B (compounds I.C.A6.B1 to I.C.A6.B21).

Table 7c

Compounds of the formula I.C in which R¹ corresponds to line A-7 of Table A and the meaning for R² for each individual compound corresponds in each case to one line of Table B (compounds I.C.A7.B1 to I.C.A7.B21).

Table 8c

Compounds of the formula I.C in which R¹ corresponds to line A-8 of Table A and the meaning for R² for each individual compound corresponds in each case to one line of Table B (compounds I.C.A8.B1 to I.C.A8.B21).

Table 9c

Compounds of the formula I.C in which R¹ corresponds to line A-9 of Table A and the meaning for R² for each individual compound corresponds in each case to one line of Table B (compounds I.C.A9.B1 to I.C.A9.B21).

Table 10c

Compounds of the formula I.C in which R¹ corresponds to line A-10 of Table A and the meaning for R² for each individual compound corresponds in each case to one line of Table B (compounds I.C.A10.B1 to I.C.A10.B21).

Table 11c

Compounds of the formula I.C in which R¹ corresponds to line A-11 of Table A and the meaning for R² for each individual compound corresponds in each case to one line of Table B (compounds I.C.A11.B1 to I.C.A11.B21).

Table 12c

Compounds of the formula I.C in which R¹ corresponds to line A-12 of Table A and the meaning for R² for each individual compound corresponds in each case to one line of Table B (compounds I.C.A12.B1 to I.C.A12.B21).

Table 13c

Compounds of the formula I.C in which R¹ corresponds to line A-13 of Table A and the meaning for R² for each individual compound corresponds in each case to one line of Table B (compounds I.C.A13.B1 to I.C.A13.B21).

Table 14c

Compounds of the formula I.C in which R¹ corresponds to line A-14 of Table A and the meaning for R² for each individual compound corresponds in each case to one line of Table B (compounds I.C.A14.B1 to I.C.A14.B21).

Table 15c

Compounds of the formula I.C in which R¹ corresponds to line A-15 of Table A and the meaning for R² for each individual compound corresponds in each case to one line of Table B (compounds I.C.A15.B1 to I.C.A15.B21).

Table 16c

Compounds of the formula I.C in which R¹ corresponds to line A-16 of Table A and the meaning for R² for each individual compound corresponds in each case to one line of Table B (compounds I.C.A16.B1 to I.C.A16.B21).

Table 17c

Compounds of the formula I.C in which R¹ corresponds to line A-17 of Table A and the meaning for R² for each individual compound corresponds in each case to one line of Table B (compounds I.C.A17.B1 to I.C.A17.B21).

Table 18c

Compounds of the formula I.C in which R¹ corresponds to line A-18 of Table A and the meaning for R² for each individual compound corresponds in each case to one line of Table B (compounds I.C.A18.B1 to I.C.A18.B21).

Table 19c

Compounds of the formula I.C in which R¹ corresponds to line A-19 of Table A and the meaning for R² for each individual compound corresponds in each case to one line of Table B (compounds I.C.A19.B1 to I.C.A19.B21).

Table 20c

Compounds of the formula I.C in which R¹ corresponds to line A-20 of Table A and the meaning for R² for each individual compound corresponds in each case to one line of Table B (compounds I.C.A20.B1 to I.C.A20.B21).

Table 21c

Compounds of the formula I.C in which R¹ corresponds to line A-21 of Table A and the meaning for R² for each individual compound corresponds in each case to one line of Table B (compounds I.C.A21.B1 to I.C.A21.B21).

Table 22c

Compounds of the formula I.C in which R¹ corresponds to line A-22 of Table A and the meaning for the combination of (R⁴)ₘ and R³² for each individual compound corresponds in each case to one line of Table B (compounds I.C.A22.B1 to I.C.A22.B21).

Table 23c

Compounds of the formula I.C in which R¹ corresponds to line A-23 of Table A and the meaning for R² for each individual compound corresponds in each case to one line of Table B (compounds I.C.A23.B1 to I.C.A23.B21).

Table 24c

Compounds of the formula I.C in which R¹ corresponds to line A-24 of Table A and the meaning for R² for each individual compound corresponds in each case to one line of Table B (compounds I.C.A24.B1 to I.C.A24.B21).

Table 25c

Compounds of the formula I.C in which R¹ corresponds to line A-25 of Table A and the meaning for R² for each individual compound corresponds in each case to one line of Table B (compounds I.C.A25.B1 to I.C.A25.B21).

Table 26c

Compounds of the formula I.C in which R¹ corresponds to line A-26 of Table A and the meaning for R² for each individual compound corresponds in each case to one line of Table B (compounds I.C.A26.B1 to I.C.A26.B21).

Table 27c

Compounds of the formula I.C in which R¹ corresponds to line A-27 of Table A and the meaning for R² for each individual compound corresponds in each case to one line of Table B (compounds I.C.A27.B1 to I.C.A27.B21).

Table 28c

Compounds of the formula I.C in which R¹ corresponds to line A-28 of Table A and the meaning for R² for each individual compound corresponds in each case to one line of Table B (compounds I.C.A28.B1 to I.C.A28.B21).

Table 29c

Compounds of the formula I.C in which R¹ corresponds to line A-29 of Table A and the meaning for R² for each individual compound corresponds in each case to one line of Table B (compounds I.C.A29.B1 to I.C.A29.B21).

Table 30c

Compounds of the formula I.C in which R¹ corresponds to line A-30 of Table A and the meaning for R² for each individual compound corresponds in each case to one line of Table B (compounds I.C.A30.B1 to I.C.A30.B21).

Table 31c

Compounds of the formula I.C in which R¹ corresponds to line A-31 of Table A and the meaning for R² for each individual compound corresponds in each case to one line of Table B (compounds I.C.A31.B1 to I.C.A31.B21).

Table 32c

Compounds of the formula I.C in which R¹ corresponds to line A-32 of Table A and the meaning for R² for each individual compound corresponds in each case to one line of Table B (compounds I.C.A32.B1 to I.C.A32.B21).

Table 33c

Compounds of the formula I.C in which R¹ corresponds to line A-33 of Table A and the meaning for R² for each individual compound corresponds in each case to one line of Table B (compounds I.C.A33.B1 to I.C.A33.B21).

Table 34c

Compounds of the formula I.C in which R¹ corresponds to line A-34 of Table A and the meaning for R² for each individual compound corresponds in each case to one line of Table B (compounds I.C.A34.B1 to I.C.A34.B21).

Table 35c

Compounds of the formula I.C in which R¹ corresponds to line A-35 of Table A and the meaning for R² for each individual compound corresponds in each case to one line of Table B (compounds I.C.A35.B1 to I.C.A35.B21).

Table 36c

Compounds of the formula I.C in which R¹ corresponds to line A-36 of Table A and the meaning for R² for each individual compound corresponds in each case to one line of Table B (compounds I.C.A36.B1 to I.C.A36.B21).

Table 37c

Compounds of the formula I.C in which R¹ corresponds to line A-37 of Table A and the meaning for R² for each individual compound corresponds in each case to one line of Table B (compounds I.C.A37.B1 to I.C.A37.B21).

Table 38c

Compounds of the formula I.C in which R¹ corresponds to line A-38 of Table A and the meaning for R² for each individual compound corresponds in each case to one line of Table B (compounds I.C.A38.B1 to I.C.A38.B21).

Table 39c

Compounds of the formula I.C in which R¹ corresponds to line A-39 of Table A and the meaning for R² for each individual compound corresponds in each case to one line of Table B (compounds I.C.A39.B1 to I.C.A39.B21).

Table 40c

Compounds of the formula I.C in which R¹ corresponds to line A-40 of Table A and the meaning for R² for each individual compound corresponds in each case to one line of Table B (compounds I.C.A40.B1 to I.C.A40.B21).

Table 41c

Compounds of the formula I.C in which R¹ corresponds to line A-41 of Table A and the meaning for R² for each individual compound corresponds in each case to one line of Table B (compounds I.C.A41.B1 to I.C.A41.B21).

Table 42c

Compounds of the formula I.C in which R¹ corresponds to line A-42 of Table A and the meaning for R² for each individual compound corresponds in each case to one line of Table B (compounds I.C.A42.B1 to I.C.A42.B21).

Table 43c

Compounds of the formula I.C in which R¹ corresponds to line A-43 of Table A and the meaning for R² for each individual compound corresponds in each case to one line of Table B (compounds I.C.A43.B1 to I.C.A43.B21).

Table 44c

Compounds of the formula I.C in which R¹ corresponds to line A-44 of Table A and the meaning for R² for each individual compound corresponds in each case to one line of Table B (compounds I.C.A44.B1 to I.C.A44.B21).

Table 45c

Compounds of the formula I.C in which R¹ corresponds to line A-45 of Table A and the meaning for R² for each individual compound corresponds in each case to one line of Table B (compounds I.C.A45.B1 to I.C.A45.B21).

Table 46c

Compounds of the formula I.C in which R¹ corresponds to line A-46 of Table A and the meaning for R² for each individual compound corresponds in each case to one line of Table B (compounds I.C.A46.B1 to I.C.A46.B21).

Table 47c

Compounds of the formula I.C in which R¹ corresponds to line A-47 of Table A and the meaning for R² for each individual compound corresponds in each case to one line of Table B (compounds I.C.A47.B1 to I.C.A47.B21).

Table 48c

Compounds of the formula I.C in which R¹ corresponds to line A-48 of Table A and the meaning for R² for each individual compound corresponds in each case to one line of Table B (compounds I.C.A48.B1 to I.C.A48.B21).

Table 49c

Compounds of the formula I.C in which R¹ corresponds to line A-49 of Table A and the meaning for R² for each individual compound corresponds in each case to one line of Table B (compounds I.C.A49.B1 to I.C.A49.B21).

Table 50c

Compounds of the formula I.C in which R¹ corresponds to line A-50 of Table A and the meaning for R² for each individual compound corresponds in each case to one line of Table B (compounds I.C.A50.B1 to I.C.A50.B21).

Table 51c

Compounds of the formula I.C in which R¹ corresponds to line A-51 of Table A and the meaning for R² for each individual compound corresponds in each case to one line of Table B (compounds I.C.A51.B1 to I.C.A51.B21).

Table 52c

Compounds of the formula I.C in which R¹ corresponds to line A-52 of Table A and the meaning for R² for each individual compound corresponds in each case to one line of Table B (compounds I.C.A52.B1 to I.C.A52.B21).

Table 53c

Compounds of the formula I.C in which R¹ corresponds to line A-53 of Table A and the meaning for R² for each individual compound corresponds in each case to one line of Table B (compounds I.C.A53.B1 to I.C.A53.B21).

Table 54c

Compounds of the formula I.C in which R¹ corresponds to line A-54 of Table A and the meaning for R² for each individual compound corresponds in each case to one line of Table B (compounds I.C.A54.B1 to I.C.A54.B21).

Table 55c

Compounds of the formula I.C in which R¹ corresponds to line A-55 of Table A and the meaning for R² for each individual compound corresponds in each case to one line of Table B (compounds I.C.A55.B1 to I.C.A55.B21).

Table 56c

Compounds of the formula I.C in which R¹ corresponds to line A-56 of Table A and the meaning for R² for each individual compound corresponds in each case to one line of Table B (compounds I.C.A56.B1 to I.C.A56.B21).

Table 57c

Compounds of the formula I.C in which R¹ corresponds to line A-57 of Table A and the meaning for R² for each individual compound corresponds in each case to one line of Table B (compounds I.C.A57.B1 to I.C.A57.B21).

Table 58c

Compounds of the formula I.C in which R¹ corresponds to line A-58 of Table A and the meaning for R² for each individual compound corresponds in each case to one line of Table B (compounds I.C.A58.B1 to I.C.A58.B21).

Table 59c

Compounds of the formula I.C in which R¹ corresponds to line A-59 of Table A and the meaning for R² for each individual compound corresponds in each case to one line of Table B (compounds I.C.A59.B1 to I.C.A59.B21).

Table 60c

Compounds of the formula I.C in which R¹ corresponds to line A-60 of Table A and the meaning for R² for each individual compound corresponds in each case to one line of Table B (compounds I.C.A60.B1 to I.C.A60.B21).

Table 61c

Compounds of the formula I.C in which R¹ corresponds to line A-61 of Table A and the meaning for R² for each individual compound corresponds in each case to one line of Table B (compounds I.C.A61.B1 to I.C.A61.B21).

Table 62c

Compounds of the formula I.C in which R¹ corresponds to line A-62 of Table A and the meaning for R² for each individual compound corresponds in each case to one line of Table B (compounds I.C.A62.B1 to I.C.A62.B21).

Table 63c

Compounds of the formula I.C in which R¹ corresponds to line A-63 of Table A and the meaning for R² for each individual compound corresponds in each case to one line of Table B (compounds I.C.A63.B1 to I.C.A63.B21).

Table 64c

Compounds of the formula I.C in which R¹ corresponds to line A-64 of Table A and the meaning for R² for each individual compound corresponds in each case to one line of Table B (compounds I.C.A64.B1 to I.C.A64.B21).

Table 65c

Compounds of the formula I.C in which R¹ corresponds to line A-65 of Table A and the meaning for R² for each individual compound corresponds in each case to one line of Table B (compounds I.C.A65.B1 to I.C.A65.B21).

Table 66c

Compounds of the formula I.C in which R¹ corresponds to line A-66 of Table A and the meaning for R² for each individual compound corresponds in each case to one line of Table B (compounds I.C.A66.B1 to I.C.A66.B21).

Table 67c

Compounds of the formula I.C in which R¹ corresponds to line A-67 of Table A and the meaning for R² for each individual compound corresponds in each case to one line of Table B (compounds I.C.A67.B1 to I.C.A67.B21).

Table 68c

Compounds of the formula I.C in which R¹ corresponds to line A-68 of Table A and the meaning for R² for each individual compound corresponds in each case to one line of Table B (compounds I.C.A68.B1 to I.C.A68.B21).

Table 69c

Compounds of the formula I.C in which R¹ corresponds to line A-69 of Table A and the meaning for R² for each individual compound corresponds in each case to one line of Table B (compounds I.C.A69.B1 to I.C.A69.B21).

Table 70c

Compounds of the formula I.C in which R¹ corresponds to line A-70 of Table A and the meaning for R² for each individual compound corresponds in each case to one line of Table B (compounds I.C.A70.B1 to I.C.A70.B21).

Table 71c

Compounds of the formula I.C in which R¹ corresponds to line A-71 of Table A and the meaning for R² for each individual compound corresponds in each case to one line of Table B (compounds I.C.A71.B1 to I.C.A71.B21).

Table 72c

Compounds of the formula I.C in which R¹ corresponds to line A-72 of Table A and the meaning for R² for each individual compound corresponds in each case to one line of Table B (compounds I.C.A72.B1 to I.C.A72.B21).

Table 73c

Compounds of the formula I.C in which R¹ corresponds to line A-73 of Table A and the meaning for R² for each individual compound corresponds in each case to one line of Table B (compounds I.C.A73.B1 to I.C.A73.B21).

Table 74c

Compounds of the formula I.C in which R¹ corresponds to line A-74 of Table A and the meaning for R² for each individual compound corresponds in each case to one line of Table B (compounds I.C.A74.B1 to I.C.A74B21).

Table 75c

Compounds of the formula I.C in which R¹ corresponds to line A-75 of Table A and the meaning for R² for each individual compound corresponds in each case to one line of Table B (compounds I.C.A75.B1 to I.C.A75.B21).

Table 76c

Compounds of the formula I.C in which R¹ corresponds to line A-76 of Table A and the meaning for R² for each individual compound corresponds in each case to one line of Table B (compounds I.C.A76.B1 to I.C.A76.B21).

Table 77c

Compounds of the formula I.C in which R¹ corresponds to line A-77 of Table A and the meaning for R² for each individual compound corresponds in each case to one line of Table B (compounds I.C.A77.B1 to I.C.A77.B21).

Table 78c

Compounds of the formula I.C in which R¹ corresponds to line A-78 of Table A and the meaning for R² for each individual compound corresponds in each case to one line of Table B (compounds I.C.A78.B1 to I.C.A78.B21).

Table 79c

Compounds of the formula I.C in which R¹ corresponds to line A-79 of Table A and the meaning for R² for each individual compound corresponds in each case to one line of Table B (compounds I.C.A79.B1 to I.C.A79.B21).

Table 80c

Compounds of the formula I.C in which R¹ corresponds to line A-80 of Table A and the meaning for R² for each individual compound corresponds in each case to one line of Table B (compounds I.C.A80.B1 to I.C.A80.B21).

Table 81c

Compounds of the formula I.C in which R¹ corresponds to line A-81 of Table A and the meaning for R² for each individual compound corresponds in each case to one line of Table B (compounds I.C.A81.B1 to I.C.A81.B21).

Table 82c

Compounds of the formula I.C in which R¹ corresponds to line A-82 of Table A and the meaning for R² for each individual compound corresponds in each case to one line of Table B (compounds I.C.A82.B1 to I.C.A82.B21).

Table 83c

Compounds of the formula I.C in which R¹ corresponds to line A-83 of Table A and the meaning for R² for each individual compound corresponds in each case to one line of Table B (compounds I.C.A83.B1 to I.C.A83.B21).

Table 84c

Compounds of the formula I.C in which R¹ corresponds to line A-84 of Table A and the meaning for R² for each individual compound corresponds in each case to one line of Table B (compounds I.C.A84.B1 to I.C.A84.B21).

Table 85c

Compounds of the formula I.C in which R¹ corresponds to line A-85 of Table A and the meaning for R² for each individual compound corresponds in each case to one line of Table B (compounds I.C.A85.B1 to I.C.A85.B21).

Table 86c

Compounds of the formula I.C in which R¹ corresponds to line A-86 of Table A and the meaning for R² for each individual compound corresponds in each case to one line of Table B (compounds I.C.A86.B1 to I.C.A86.B21).

Table 87c

Compounds of the formula I.C in which R¹ corresponds to line A-87 of Table A and the meaning for R² for each individual compound corresponds in each case to one line of Table B (compounds I.C.A87.B1 to I.C.A87.B21).

Table 88c

Compounds of the formula I.C in which R¹ corresponds to line A-88 of Table A and the meaning for R² for each individual compound corresponds in each case to one line of Table B (compounds I.C.A88.B1 to I.C.A88.B21).

Table 89c

Compounds of the formula I.C in which R¹ corresponds to line A-89 of Table A and the meaning for R² for each individual compound corresponds in each case to one line of Table B (compounds I.C.A89.B1 to I.C.A89.B21).

Table 90c

Compounds of the formula I.C in which R¹ corresponds to line A-90 of Table A and the meaning for R² for each individual compound corresponds in each case to one line of Table B (compounds I.C.A90.B1 to I.C.A90.B21).

Table 91c

Compounds of the formula I.C in which R¹ corresponds to line A-91 of Table A and the meaning for R² for each individual compound corresponds in each case to one line of Table B (compounds I.C.A91.B1 to I.C.A91.B21).

Table 92c

Compounds of the formula I.C in which R¹ corresponds to line A-92 of Table A and the meaning for R² for each individual compound corresponds in each case to one line of Table B (compounds I.C.A92.B1 to I.C.A92.B21).

Table 93c

Compounds of the formula I.C in which R¹ corresponds to line A-93 of Table A and the meaning for R² for each individual compound corresponds in each case to one line of Table B (compounds I.C.A93.B1 to I.C.A93.B21).

Table 94c

Compounds of the formula I.C in which R¹ corresponds to line A-94 of Table A and the meaning for R² for each individual compound corresponds in each case to one line of Table B (compounds I.C.A94.B1 to I.C.A94.B21).

Table 95c

Compounds of the formula I.C in which R¹ corresponds to line A-95 of Table A and the meaning for R² for each individual compound corresponds in each case to one line of Table B (compounds I.C.A95.B1 to I.C.A95.B21).

Table 96c

Compounds of the formula I.C in which R¹ corresponds to line A-96 of Table A and the meaning for R² for each individual compound corresponds in each case to one line of Table B (compounds I.C.A96.B1 to I.C.A96.B21).

Table 97c

Compounds of the formula I.C in which R¹ corresponds to line A-97 of Table A and the meaning for R² for each individual compound corresponds in each case to one line of Table B (compounds I.C.A97.B1 to I.C.A97.B21).

Table 98c

Compounds of the formula I.C in which R¹ corresponds to line A-98 of Table A and the meaning for R² for each individual compound corresponds in each case to one line of Table B (compounds I.C.A98.B1 to I.C.A98.B21).

Table 99c

Compounds of the formula I.C in which R¹ corresponds to line A-99 of Table A and the meaning for R² for each individual compound corresponds in each case to one line of Table B (compounds I.C.A99.B1 to I.C.A99.B21).

Table 100c

Compounds of the formula I.C in which R¹ corresponds to line A-100 of Table A and the meaning for R² for each individual compound corresponds in each case to one line of Table B (compounds I.C.A100.B1 to I.C.A100.B21).

Table 101c

Compounds of the formula I.C in which R¹ corresponds to line A-101 of Table A and the meaning for R² for each individual compound corresponds in each case to one line of Table B (compounds I.C.A101.B1 to I.C.A101.B21).

Table 102c

Table 1d

Compounds of the formula I.D in which R¹ corresponds to line A-1 of Table A and the meaning R² for each individual compound corresponds in each case to one line of Table B (compounds I.D.A1.B1 to I.D.A1.B21).

Table 2d

Compounds of the formula I.D in which R¹ corresponds to line A-2 of Table A and the meaning for R² for each individual compound corresponds in each case to one line of Table B (compounds I.D.A2.B1 to I.D.A2.B21).

Table 3d

Compounds of the formula I.D in which R¹ corresponds to line A-3 of Table A and the meaning for R² for each individual compound corresponds in each case to one line of Table B (compounds I.D.A3.B1 to I.D.A3.B21).

Table 4d

Compounds of the formula I.D in which R¹ corresponds to line A-4 of Table A and the meaning for R² for each individual compound corresponds in each case to one line of Table B (compounds I.D.A4.B1 to I.D.A4.B21).

Table 5d

Compounds of the formula I.D in which R¹ corresponds to line A-5 of Table A and the meaning for R² for each individual compound corresponds in each case to one line of Table B (compounds I.D.A5.B1 to I.D.A5.B21).

Table 6d

Compounds of the formula I.D in which R¹ corresponds to line A-6 of Table A and the meaning for R² for each individual compound corresponds in each case to one line of Table B (compounds I.D.A6.B1 to I.D.A6.B21).

Table 7d

Compounds of the formula I.D in which R¹ corresponds to line A-7 of Table A and the meaning for R² for each individual compound corresponds in each case to one line of Table B (compounds I.D.A7.B1 to I.D.A7.B21).

Table 8d

Compounds of the formula I.D in which R¹ corresponds to line A-8 of Table A and the meaning for R² for each individual compound corresponds in each case to one line of Table B (compounds I.D.A8.B1 to I.D.A8.B21).

Table 9d

Compounds of the formula I.D in which R¹ corresponds to line A-9 of Table A and the meaning for R² for each individual compound corresponds in each case to one line of Table B (compounds I.D.A9.B1 to I.D.A9.B21).

Table 10d

Compounds of the formula I.D in which R¹ corresponds to line A-10 of Table A and the meaning for R² for each individual compound corresponds in each case to one line of Table B (compounds I.D.A10.B1 to I.D.A10.B21).

Table 11d

Compounds of the formula I.D in which R¹ corresponds to line A-11 of Table A and the meaning for R² for each individual compound corresponds in each case to one line of Table B (compounds I.D.A11.B1 to I.D.A11.B21).

Table 12d

Compounds of the formula I.D in which R¹ corresponds to line A-12 of Table A and the meaning for R² for each individual compound corresponds in each case to one line of Table B (compounds I.D.A12.B1 to I.D.A12.B21).

Table 13d

Compounds of the formula I.D in which R¹ corresponds to line A-13 of Table A and the meaning for R² for each individual compound corresponds in each case to one line of Table B (compounds I.D.A13.B1 to I.D.A13.B21).

Table 14d

Compounds of the formula I.D in which R¹ corresponds to line A-14 of Table A and the meaning for R² for each individual compound corresponds in each case to one line of Table B (compounds I.D.A14.B1 to I.D.A14.B21).

Table 15d

Compounds of the formula I.D in which R¹ corresponds to line A-15 of Table A and the meaning for R² for each individual compound corresponds in each case to one line of Table B (compounds I.D.A15.B1 to I.D.A15.B21).

Table 16d

Compounds of the formula I.D in which R¹ corresponds to line A-16 of Table A and the meaning for R² for each individual compound corresponds in each case to one line of Table B (compounds I.D.A16.B1 to I.D.A16.B21).

Table 17d

Compounds of the formula I.D in which R¹ corresponds to line A-17 of Table A and the meaning for R² for each individual compound corresponds in each case to one line of Table B (compounds I.D.A17.B1 to I.D.A17.B21).

Table 18d

Compounds of the formula I.D in which R¹ corresponds to line A-18 of Table A and the meaning for R² for each individual compound corresponds in each case to one line of Table B (compounds I.D.A18.B1 to I.D.A18.B21).

Table 19d

Compounds of the formula I.D in which R¹ corresponds to line A-19 of Table A and the meaning for R² for each individual compound corresponds in each case to one line of Table B (compounds I.D.A19.B1 to I.D.A19.B21).

Table 20d

Compounds of the formula I.D in which R¹ corresponds to line A-20 of Table A and the meaning for R² for each individual compound corresponds in each case to one line of Table B (compounds I.D.A20.B1 to I.D.A20.B21).

Table 21d

Compounds of the formula I.D in which R¹ corresponds to line A-21 of Table A and the meaning for R² for each individual compound corresponds in each case to one line of Table B (compounds I.D.A21.B1 to I.D.A21.B21).

Table 22d

Compounds of the formula I.D in which R¹ corresponds to line A-22 of Table A and the meaning for the combination of (R⁴)ₘ and R³² for each individual compound corresponds in each case to one line of Table B (compounds I.D.A22.B1 to I.D.A22.B21).

Table 23d

Compounds of the formula I.D in which R¹ corresponds to line A-23 of Table A and the meaning for R² for each individual compound corresponds in each case to one line of Table B (compounds I.D.A23.B1 to I.D.A23.B21).

Table 24d

Compounds of the formula I.D in which R¹ corresponds to line A-24 of Table A and the meaning for R² for each individual compound corresponds in each case to one line of Table B (compounds I.D.A24.B1 to I.D.A24.B21).

Table 25d

Compounds of the formula I.D in which R¹ corresponds to line A-25 of Table A and the meaning for R² for each individual compound corresponds in each case to one line of Table B (compounds I.D.A25.B1 to I.D.A25.B21).

Table 26d

Compounds of the formula I.D in which R¹ corresponds to line A-26 of Table A and the meaning for R² for each individual compound corresponds in each case to one line of Table B (compounds I.D.A26.B1 to I.D.A26.B21).

Table 27d

Compounds of the formula I.D in which R¹ corresponds to line A-27 of Table A and the meaning for R² for each individual compound corresponds in each case to one line of Table B (compounds I.D.A27.B1 to I.D.A27.B21).

Table 28d

Compounds of the formula I.D in which R¹ corresponds to line A-28 of Table A and the meaning for R² for each individual compound corresponds in each case to one line of Table B (compounds I.D.A28.B1 to I.D.A28.B21).

Table 29d

Compounds of the formula I.D in which R¹ corresponds to line A-29 of Table A and the meaning for R² for each individual compound corresponds in each case to one line of Table B (compounds I.D.A29.B1 to I.D.A29.B21).

Table 30d

Compounds of the formula I.D in which R¹ corresponds to line A-30 of Table A and the meaning for R² for each individual compound corresponds in each case to one line of Table B (compounds I.D.A30.B1 to I.D.A30.B21).

Table 31d

Compounds of the formula I.D in which R¹ corresponds to line A-31 of Table A and the meaning for R² for each individual compound corresponds in each case to one line of Table B (compounds I.D.A31.B1 to I.D.A31.B21).

Table 32d

Compounds of the formula I.D in which R¹ corresponds to line A-32 of Table A and the meaning for R² for each individual compound corresponds in each case to one line of Table B (compounds I.D.A32.B1 to I.D.A32.B21).

Table 33d

Compounds of the formula I.D in which R¹ corresponds to line A-33 of Table A and the meaning for R² for each individual compound corresponds in each case to one line of Table B (compounds I.D.A33.B1 to I.D.A33.B21).

Table 34d

Compounds of the formula I.D in which R¹ corresponds to line A-34 of Table A and the meaning for R² for each individual compound corresponds in each case to one line of Table B (compounds I.D.A34.B1 to I.D.A34.B21).

Table 35d

Compounds of the formula I.D in which R¹ corresponds to line A-35 of Table A and the meaning for R² for each individual compound corresponds in each case to one line of Table B (compounds I.D.A35.B1 to I.D.A35.B21).

Table 36d

Compounds of the formula I.D in which R¹ corresponds to line A-36 of Table A and the meaning for R² for each individual compound corresponds in each case to one line of Table B (compounds I.D.A36.B1 to I.D.A36.B21).

Table 37d

Compounds of the formula I.D in which R¹ corresponds to line A-37 of Table A and the meaning for R² for each individual compound corresponds in each case to one line of Table B (compounds I.D.A37.B1 to I.D.A37.B21).

Table 38d

Compounds of the formula I.D in which R¹ corresponds to line A-38 of Table A and the meaning for R² for each individual compound corresponds in each case to one line of Table B (compounds I.D.A38.B1 to I.D.A38.B21).

Table 39d

Compounds of the formula I.D in which R¹ corresponds to line A-39 of Table A and the meaning for R² for each individual compound corresponds in each case to one line of Table B (compounds I.D.A39.B1 to I.D.A39.B21).

Table 40d

Compounds of the formula I.D in which R¹ corresponds to line A-40 of Table A and the meaning for R² for each individual compound corresponds in each case to one line of Table B (compounds I.D.A40.B1 to I.D.A40.B21).

Table 41d

Compounds of the formula I.D in which R¹ corresponds to line A-41 of Table A and the meaning for R² for each individual compound corresponds in each case to one line of Table B (compounds I.D.A41.B1 to I.D.A41.B21).

Table 42d

Compounds of the formula I.D in which R¹ corresponds to line A-42 of Table A and the meaning for R² for each individual compound corresponds in each case to one line of Table B (compounds I.D.A42.B1 to I.D.A42.B21).

Table 43d

Compounds of the formula I.D in which R¹ corresponds to line A-43 of Table A and the meaning for R² for each individual compound corresponds in each case to one line of Table B (compounds I.D.A43.B1 to I.D.A43.B21).

Table 44d

Compounds of the formula I.D in which R¹ corresponds to line A-44 of Table A and the meaning for R² for each individual compound corresponds in each case to one line of Table B (compounds I.D.A44.B1 to I.D.A44.B21).

Table 45d

Compounds of the formula I.D in which R¹ corresponds to line A-45 of Table A and the meaning for R² for each individual compound corresponds in each case to one line of Table B (compounds I.D.A45.B1 to I.D.A45.B21).

Table 46d

Compounds of the formula I.D in which R¹ corresponds to line A-46 of Table A and the meaning for R² for each individual compound corresponds in each case to one line of Table B (compounds I.D.A46.B1 to I.D.A46.B21).

Table 47d

Compounds of the formula I.D in which R¹ corresponds to line A-47 of Table A and the meaning for R² for each individual compound corresponds in each case to one line of Table B (compounds I.D.A47.B1 to I.D.A47.B21).

Table 48d

Compounds of the formula I.D in which R¹ corresponds to line A-48 of Table A and the meaning for R² for each individual compound corresponds in each case to one line of Table B (compounds I.D.A48.B1 to I.D.A48.B21).

Table 49d

Compounds of the formula I.D in which R¹ corresponds to line A-49 of Table A and the meaning for R² for each individual compound corresponds in each case to one line of Table B (compounds I.D.A49.B1 to I.D.A49.B21).

Table 50d

Compounds of the formula I.D in which R¹ corresponds to line A-50 of Table A and the meaning for R² for each individual compound corresponds in each case to one line of Table B (compounds I.D.A50.B1 to I.D.A50.B21).

Table 51d

Compounds of the formula I.D in which R¹ corresponds to line A-51 of Table A and the meaning for R² for each individual compound corresponds in each case to one line of Table B (compounds I.D.A51.B1 to I.D.A51.B21).

Table 52d

Compounds of the formula I.D in which R¹ corresponds to line A-52 of Table A and the meaning for R² for each individual compound corresponds in each case to one line of Table B (compounds I.D.A52.B1 to I.D.A52.B21).

Table 53d

Compounds of the formula I.D in which R¹ corresponds to line A-53 of Table A and the meaning for R² for each individual compound corresponds in each case to one line of Table B (compounds I.D.A53.B1 to I.D.A53.B21).

Table 54d

Compounds of the formula I.D in which R¹ corresponds to line A-54 of Table A and the meaning for R² for each individual compound corresponds in each case to one line of Table B (compounds I.D.A54.B1 to I.D.A54.B21).

Table 55d

Compounds of the formula I.D in which R¹ corresponds to line A-55 of Table A and the meaning for R² for each individual compound corresponds in each case to one line of Table B (compounds I.D.A55.B1 to I.D.A55.B21).

Table 56d

Compounds of the formula I.D in which R¹ corresponds to line A-56 of Table A and the meaning for R² for each individual compound corresponds in each case to one line of Table B (compounds I.D.A56.B1 to I.D.A56.B21).

Table 57d

Compounds of the formula I.D in which R¹ corresponds to line A-57 of Table A and the meaning for R² for each individual compound corresponds in each case to one line of Table B (compounds I.D.A57.B1 to I.D.A57.B21).

Table 58d

Compounds of the formula I.D in which R¹ corresponds to line A-58 of Table A and the meaning for R² for each individual compound corresponds in each case to one line of Table B (compounds I.D.A58.B1 to I.D.A58.B21).

Table 59d

Compounds of the formula I.D in which R¹ corresponds to line A-59 of Table A and the meaning for R² for each individual compound corresponds in each case to one line of Table B (compounds I.D.A59.B1 to I.D.A59.B21).

Table 60d

Compounds of the formula I.D in which R¹ corresponds to line A-60 of Table A and the meaning for R² for each individual compound corresponds in each case to one line of Table B (compounds I.D.A60.B1 to I.D.A60.B21).

Table 61d

Compounds of the formula I.D in which R¹ corresponds to line A-61 of Table A and the meaning for R² for each individual compound corresponds in each case to one line of Table B (compounds I.D.A61.B1 to I.D.A61.B21).

Table 62d

Compounds of the formula I.D in which R¹ corresponds to line A-62 of Table A and the meaning for R² for each individual compound corresponds in each case to one line of Table B (compounds I.D.A62.B1 to I.D.A62.B21).

Table 63d

Compounds of the formula I.D in which R¹ corresponds to line A-63 of Table A and the meaning for R² for each individual compound corresponds in each case to one line of Table B (compounds I.D.A63.B1 to I.D.A63.B21).

Table 64d

Compounds of the formula I.D in which R¹ corresponds to line A-64 of Table A and the meaning for R² for each individual compound corresponds in each case to one line of Table B (compounds I.D.A64.B1 to I.D.A64.B21).

Table 65d

Compounds of the formula I.D in which R¹ corresponds to line A-65 of Table A and the meaning for R² for each individual compound corresponds in each case to one line of Table B (compounds I.D.A65.B1 to I.D.A65.B21).

Table 66d

Compounds of the formula I.D in which R¹ corresponds to line A-66 of Table A and the meaning for R² for each individual compound corresponds in each case to one line of Table B (compounds I.D.A66.B1 to I.D.A66.B21).

Table 67d

Compounds of the formula I.D in which R¹ corresponds to line A-67 of Table A and the meaning for R² for each individual compound corresponds in each case to one line of Table B (compounds I.D.A67.B1 to I.D.A67.B21).

Table 68d

Compounds of the formula I.D in which R¹ corresponds to line A-68 of Table A and the meaning for R² for each individual compound corresponds in each case to one line of Table B (compounds I.D.A68.B1 to I.D.A68.B21).

Table 69d

Compounds of the formula I.D in which R¹ corresponds to line A-69 of Table A and the meaning for R² for each individual compound corresponds in each case to one line of Table B (compounds I.D.A69.B1 to I.D.A69.B21).

Table 70d

Compounds of the formula I.D in which R¹ corresponds to line A-70 of Table A and the meaning for R² for each individual compound corresponds in each case to one line of Table B (compounds I.D.A70.B1 to I.D.A70.B21).

Table 71d

Compounds of the formula I.D in which R¹ corresponds to line A-71 of Table A and the meaning for R² for each individual compound corresponds in each case to one line of Table B (compounds I.D.A71.B1 to I.D.A71.B21).

Table 72d

Compounds of the formula I.D in which R¹ corresponds to line A-72 of Table A and the meaning for R² for each individual compound corresponds in each case to one line of Table B (compounds I.D.A72.B1 to I.D.A72.B21).

Table 73d

Compounds of the formula I.D in which R¹ corresponds to line A-73 of Table A and the meaning for R² for each individual compound corresponds in each case to one line of Table B (compounds I.D.A73.B1 to I.D.A73.B21).

Table 74d

Compounds of the formula I.D in which R¹ corresponds to line A-74 of Table A and the meaning for R² for each individual compound corresponds in each case to one line of Table B (compounds I.D.A74.B1 to I.D.A74B21).

Table 75d

Compounds of the formula I.D in which R¹ corresponds to line A-75 of Table A and the meaning for R² for each individual compound corresponds in each case to one line of Table B (compounds I.D.A75.B1 to I.D.A75.B21).

Table 76d

Compounds of the formula I.D in which R¹ corresponds to line A-76 of Table A and the meaning for R² for each individual compound corresponds in each case to one line of Table B (compounds I.D.A76.B1 to I.D.A76.B21).

Table 77d

Compounds of the formula I.D in which R¹ corresponds to line A-77 of Table A and the meaning for R² for each individual compound corresponds in each case to one line of Table B (compounds I.D.A77.B1 to I.D.A77.B21).

Table 78d

Compounds of the formula I.D in which R¹ corresponds to line A-78 of Table A and the meaning for R² for each individual compound corresponds in each case to one line of Table B (compounds I.D.A78.B1 to I.D.A78.B21).

Table 79d

Compounds of the formula I.D in which R¹ corresponds to line A-79 of Table A and the meaning for R² for each individual compound corresponds in each case to one line of Table B (compounds I.D.A79.B1 to I.D.A79.B21).

Table 80d

Compounds of the formula I.D in which R¹ corresponds to line A-80 of Table A and the meaning for R² for each individual compound corresponds in each case to one line of Table B (compounds I.D.A80.B1 to I.D.A80.B21).

Table 81d

Compounds of the formula I.D in which R¹ corresponds to line A-81 of Table A and the meaning for R² for each individual compound corresponds in each case to one line of Table B (compounds I.D.A81.B1 to I.D.A81.B21).

Table 82d

Compounds of the formula I.D in which R¹ corresponds to line A-82 of Table A and the meaning for R² for each individual compound corresponds in each case to one line of Table B (compounds I.D.A82.B1 to I.D.A82.B21).

Table 83d

Compounds of the formula I.D in which R¹ corresponds to line A-83 of Table A and the meaning for R² for each individual compound corresponds in each case to one line of Table B (compounds I.D.A83.B1 to I.D.A83.B21).

Table 84d

Compounds of the formula I.D in which R¹ corresponds to line A-84 of Table A and the meaning for R² for each individual compound corresponds in each case to one line of Table B (compounds I.D.A84.B1 to I.D.A84.B21).

Table 85d

Compounds of the formula I.D in which R¹ corresponds to line A-85 of Table A and the meaning for R² for each individual compound corresponds in each case to one line of Table B (compounds I.D.A85.B1 to I.D.A85.B21).

Table 86d

Compounds of the formula I.D in which R¹ corresponds to line A-86 of Table A and the meaning for R² for each individual compound corresponds in each case to one line of Table B (compounds I.D.A86.B1 to I.D.A86.B21).

Table 87d

Compounds of the formula I.D in which R¹ corresponds to line A-87 of Table A and the meaning for R² for each individual compound corresponds in each case to one line of Table B (compounds I.D.A87.B1 to I.D.A87.B21).

Table 88d

Compounds of the formula I.D in which R¹ corresponds to line A-88 of Table A and the meaning for R² for each individual compound corresponds in each case to one line of Table B (compounds I.D.A88.B1 to I.D.A88.B21).

Table 89d

Compounds of the formula I.D in which R¹ corresponds to line A-89 of Table A and the meaning for R² for each individual compound corresponds in each case to one line of Table B (compounds I.D.A89.B1 to I.D.A89.B21).

Table 90d

Compounds of the formula I.D in which R¹ corresponds to line A-90 of Table A and the meaning for R² for each individual compound corresponds in each case to one line of Table B (compounds I.D.A90.B1 to I.D.A90.B21).

Table 91d

Compounds of the formula I.D in which R¹ corresponds to line A-91 of Table A and the meaning for R² for each individual compound corresponds in each case to one line of Table B (compounds I.D.A91.B1 to I.D.A91.B21).

Table 92d

Compounds of the formula I.D in which R¹ corresponds to line A-92 of Table A and the meaning for R² for each individual compound corresponds in each case to one line of Table B (compounds I.D.A92.B1 to I.D.A92.B21).

Table 93d

Compounds of the formula I.D in which R¹ corresponds to line A-93 of Table A and the meaning for R² for each individual compound corresponds in each case to one line of Table B (compounds I.D.A93.B1 to I.D.A93.B21).

Table 94d

Compounds of the formula I.D in which R¹ corresponds to line A-94 of Table A and the meaning for R² for each individual compound corresponds in each case to one line of Table B (compounds I.D.A94.B1 to I.D.A94.B21).

Table 95d

Compounds of the formula I.D in which R¹ corresponds to line A-95 of Table A and the meaning for R² for each individual compound corresponds in each case to one line of Table B (compounds I.D.A95.B1 to I.D.A95.B21).

Table 96d

Compounds of the formula I.D in which R¹ corresponds to line A-96 of Table A and the meaning for R² for each individual compound corresponds in each case to one line of Table B (compounds I.D.A96.B1 to I.D.A96.B21).

Table 97d

Compounds of the formula I.D in which R¹ corresponds to line A-97 of Table A and the meaning for R² for each individual compound corresponds in each case to one line of Table B (compounds I.D.A97.B1 to I.D.A97.B21).

Table 98d

Compounds of the formula I.D in which R¹ corresponds to line A-98 of Table A and the meaning for R² for each individual compound corresponds in each case to one line of Table B (compounds I.D.A98.B1 to I.D.A98.B21).

Table 99d

Compounds of the formula I.D in which R¹ corresponds to line A-99 of Table A and the meaning for R² for each individual compound corresponds in each case to one line of Table B (compounds I.D.A99.B1 to I.D.A99.B21).

Table 100d

Compounds of the formula I.D in which R¹ corresponds to line A-100 of Table A and the meaning for R² for each individual compound corresponds in each case to one line of Table B (compounds I.D.A100.B1 to I.D.A100.B21).

Table 101d

Compounds of the formula I.D in which R¹ corresponds to line A-101 of Table A and the meaning for R² for each individual compound corresponds in each case to one line of Table B (compounds I.D.A101.B1 to I.D.A101.B21).

Table 102d

Table 1e

Compounds of the formula I.E in which R¹ corresponds to line A-1 of Table A and the meaning R² for each individual compound corresponds in each case to one line of Table B (compounds I.E.A1.B1 to I.E.A1.B21).

Table 2e

Compounds of the formula I.E in which R¹ corresponds to line A-2 of Table A and the meaning for R² for each individual compound corresponds in each case to one line of Table B (compounds I.E.A2.B1 to I.E.A2.B21).

Table 3e

Compounds of the formula I.E in which R¹ corresponds to line A-3 of Table A and the meaning for R² for each individual compound corresponds in each case to one line of Table B (compounds I.E.A3.B1 to I.E.A3.B21).

Table 4e

Compounds of the formula I.E in which R¹ corresponds to line A-4 of Table A and the meaning for R² for each individual compound corresponds in each case to one line of Table B (compounds I.E.A4.B1 to I.E.A4.B21).

Table 5e

Compounds of the formula I.E in which R¹ corresponds to line A-5 of Table A and the meaning for R² for each individual compound corresponds in each case to one line of Table B (compounds I.E.A5.B1 to I.E.A5.B21).

Table 6e

Compounds of the formula I.E in which R¹ corresponds to line A-6 of Table A and the meaning for R² for each individual compound corresponds in each case to one line of Table B (compounds I.E.A6.B1 to I.E.A6.B21).

Table 7e

Compounds of the formula I.E in which R¹ corresponds to line A-7 of Table A and the meaning for R² for each individual compound corresponds in each case to one line of Table B (compounds I.E.A7.B1 to I.E.A7.B21).

Table 8e

Compounds of the formula I.E in which R¹ corresponds to line A-8 of Table A and the meaning for R² for each individual compound corresponds in each case to one line of Table B (compounds I.E.A8.B1 to I.E.A8.B21).

Table 9e

Compounds of the formula I.E in which R¹ corresponds to line A-9 of Table A and the meaning for R² for each individual compound corresponds in each case to one line of Table B (compounds I.E.A9.B1 to I.E.A9.B21).

Table 10e

Compounds of the formula I.E in which R¹ corresponds to line A-10 of Table A and the meaning for R² for each individual compound corresponds in each case to one line of Table B (compounds I.E.A10.B1 to I.E.A10.B21).

Table 11e

Compounds of the formula I.E in which R¹ corresponds to line A-11 of Table A and the meaning for R² for each individual compound corresponds in each case to one line of Table B (compounds I.E.A11.B1 to I.E.A11.B21).

Table 12e

Compounds of the formula I.E in which R¹ corresponds to line A-12 of Table A and the meaning for R² for each individual compound corresponds in each case to one line of Table B (compounds I.E.A12.B1 to I.E.A12.B21).

Table 13e

Compounds of the formula I.E in which R¹ corresponds to line A-13 of Table A and the meaning for R² for each individual compound corresponds in each case to one line of Table B (compounds I.E.A13.B1 to I.E.A13.B21).

Table 14e

Compounds of the formula I.E in which R¹ corresponds to line A-14 of Table A and the meaning for R² for each individual compound corresponds in each case to one line of Table B (compounds I.E.A14.B1 to I.E.A14.B21).

Table 15e

Compounds of the formula I.E in which R¹ corresponds to line A-15 of Table A and the meaning for R² for each individual compound corresponds in each case to one line of Table B (compounds I.E.A15.B1 to I.E.A15.B21).

Table 16e

Compounds of the formula I.E in which R¹ corresponds to line A-16 of Table A and the meaning for R² for each individual compound corresponds in each case to one line of Table B (compounds I.E.A16.B1 to I.E.A16.B21).

Table 17e

Compounds of the formula I.E in which R¹ corresponds to line A-17 of Table A and the meaning for R² for each individual compound corresponds in each case to one line of Table B (compounds I.E.A17.B1 to I.E.A17.B21).

Table 18e

Compounds of the formula I.E in which R¹ corresponds to line A-18 of Table A and the meaning for R² for each individual compound corresponds in each case to one line of Table B (compounds I.E.A18.B1 to I.E.A18.B21).

Table 19e

Compounds of the formula I.E in which R¹ corresponds to line A-19 of Table A and the meaning for R² for each individual compound corresponds in each case to one line of Table B (compounds I.E.A19.B1 to I.E.A19.B21).

Table 20e

Compounds of the formula I.E in which R¹ corresponds to line A-20 of Table A and the meaning for R² for each individual compound corresponds in each case to one line of Table B (compounds I.E.A20.B1 to I.E.A20.B21).

Table 21e

Compounds of the formula I.E in which R¹ corresponds to line A-21 of Table A and the meaning for R² for each individual compound corresponds in each case to one line of Table B (compounds I.E.A21.B1 to I.E.A21.B21).

Table 22e

Compounds of the formula I.E in which R¹ corresponds to line A-22 of Table A and the meaning for the combination of (R⁴)ₘ and R³² for each individual compound corresponds in each case to one line of Table B (compounds I.E.A22.B1 to I.E.A22.B21).

Table 23e

Compounds of the formula I.E in which R¹ corresponds to line A-23 of Table A and the meaning for R² for each individual compound corresponds in each case to one line of Table B (compounds I.E.A23.B1 to I.E.A23.B21).

Table 24e

Compounds of the formula I.E in which R¹ corresponds to line A-24 of Table A and the meaning for R² for each individual compound corresponds in each case to one line of Table B (compounds I.E.A24.B1 to I.E.A24.B21).

Table 25e

Compounds of the formula I.E in which R¹ corresponds to line A-25 of Table A and the meaning for R² for each individual compound corresponds in each case to one line of Table B (compounds I.E.A25.B1 to I.E.A25.B21).

Table 26e

Compounds of the formula I.E in which R¹ corresponds to line A-26 of Table A and the meaning for R² for each individual compound corresponds in each case to one line of Table B (compounds I.E.A26.B1 to I.E.A26.B21).

Table 27e

Compounds of the formula I.E in which R¹ corresponds to line A-27 of Table A and the meaning for R² for each individual compound corresponds in each case to one line of Table B (compounds I.E.A27.B1 to I.E.A27.B21).

Table 28e

Compounds of the formula I.E in which R¹ corresponds to line A-28 of Table A and the meaning for R² for each individual compound corresponds in each case to one line of Table B (compounds I.E.A28.B1 to I.E.A28.B21).

Table 29e

Compounds of the formula I.E in which R¹ corresponds to line A-29 of Table A and the meaning for R² for each individual compound corresponds in each case to one line of Table B (compounds I.E.A29.B1 to I.E.A29.B21).

Table 30e

Compounds of the formula I.E in which R¹ corresponds to line A-30 of Table A and the meaning for R² for each individual compound corresponds in each case to one line of Table B (compounds I.E.A30.B1 to I.E.A30.B21).

Table 31e

Compounds of the formula I.E in which R¹ corresponds to line A-31 of Table A and the meaning for R² for each individual compound corresponds in each case to one line of Table B (compounds I.E.A31.B1 to I.E.A31.B21).

Table 32e

Compounds of the formula I.E in which R¹ corresponds to line A-32 of Table A and the meaning for R² for each individual compound corresponds in each case to one line of Table B (compounds I.E.A32.B1 to I.E.A32.B21).

Table 33e

Compounds of the formula I.E in which R¹ corresponds to line A-33 of Table A and the meaning for R² for each individual compound corresponds in each case to one line of Table B (compounds I.E.A33.B1 to I.E.A33.B21).

Table 34e

Compounds of the formula I.E in which R¹ corresponds to line A-34 of Table A and the meaning for R² for each individual compound corresponds in each case to one line of Table B (compounds I.E.A34.B1 to I.E.A34.B21).

Table 35e

Compounds of the formula I.E in which R¹ corresponds to line A-35 of Table A and the meaning for R² for each individual compound corresponds in each case to one line of Table B (compounds I.E.A35.B1 to I.E.A35.B21).

Table 36e

Compounds of the formula I.E in which R¹ corresponds to line A-36 of Table A and the meaning for R² for each individual compound corresponds in each case to one line of Table B (compounds I.E.A36.B1 to I.E.A36.B21).

Table 37e

Compounds of the formula I.E in which R¹ corresponds to line A-37 of Table A and the meaning for R² for each individual compound corresponds in each case to one line of Table B (compounds I.E.A37.B1 to I.E.A37.B21).

Table 38e

Compounds of the formula I.E in which R¹ corresponds to line A-38 of Table A and the meaning for R² for each individual compound corresponds in each case to one line of Table B (compounds I.E.A38.B1 to I.E.A38.B21).

Table 39e

Compounds of the formula I.E in which R¹ corresponds to line A-39 of Table A and the meaning for R² for each individual compound corresponds in each case to one line of Table B (compounds I.E.A39.B1 to I.E.A39.B21).

Table 40e

Compounds of the formula I.E in which R¹ corresponds to line A-40 of Table A and the meaning for R² for each individual compound corresponds in each case to one line of Table B (compounds I.E.A40.B1 to I.E.A40.B21).

Table 41e

Compounds of the formula I.E in which R¹ corresponds to line A-41 of Table A and the meaning for R² for each individual compound corresponds in each case to one line of Table B (compounds I.E.A41.B1 to I.E.A41.B21).

Table 42e

Compounds of the formula I.E in which R¹ corresponds to line A-41 of Table A and the meaning for R² for each individual compound corresponds in each case to one line of Table B (compounds I.E.A42.B1 to I.E.A42.B21).

Table 43e

Compounds of the formula I.E in which R¹ corresponds to line A-43 of Table A and the meaning for R² for each individual compound corresponds in each case to one line of Table B (compounds I.E.A43.B1 to I.E.A43.B21).

Table 44e

Compounds of the formula I.E in which R¹ corresponds to line A-44 of Table A and the meaning for R² for each individual compound corresponds in each case to one line of Table B (compounds I.E.A44.B1 to I.E.A44.B21).

Table 45e

Compounds of the formula I.E in which R¹ corresponds to line A-45 of Table A and the meaning for R² for each individual compound corresponds in each case to one line of Table B (compounds I.E.A45.B1 to I.E.A45.B21).

Table 46e

Compounds of the formula I.E in which R¹ corresponds to line A-46 of Table A and the meaning for R² for each individual compound corresponds in each case to one line of Table B (compounds I.E.A46.B1 to I.E.A46.B21).

Table 47e

Compounds of the formula I.E in which R¹ corresponds to line A-47 of Table A and the meaning for R² for each individual compound corresponds in each case to one line of Table B (compounds I.E.A47.B1 to I.E.A47.B21).

Table 48e

Compounds of the formula I.E in which R¹ corresponds to line A-48 of Table A and the meaning for R² for each individual compound corresponds in each case to one line of Table B (compounds I.E.A48.B1 to I.E.A48.B21).

Table 49e

Compounds of the formula I.E in which R¹ corresponds to line A-49 of Table A and the meaning for R² for each individual compound corresponds in each case to one line of Table B (compounds I.E.A49.B1 to I.E.A49.B21).

Table 50e

Compounds of the formula I.E in which R¹ corresponds to line A-50 of Table A and the meaning for R² for each individual compound corresponds in each case to one line of Table B (compounds I.E.A50.B1 to I.E.A50.B21).

Table 51e

Compounds of the formula I.E in which R¹ corresponds to line A-51 of Table A and the meaning for R² for each individual compound corresponds in each case to one line of Table B (compounds I.E.A51.B1 to I.E.A51.B21).

Table 52e

Compounds of the formula I.E in which R¹ corresponds to line A-52 of Table A and the meaning for R² for each individual compound corresponds in each case to one line of Table B (compounds I.E.A52.B1 to I.E.A52.B21).

Table 53e

Compounds of the formula I.E in which R¹ corresponds to line A-53 of Table A and the meaning for R² for each individual compound corresponds in each case to one line of Table B (compounds I.E.A53.B1 to I.E.A53.B21).

Table 54e

Compounds of the formula I.E in which R¹ corresponds to line A-54 of Table A and the meaning for R² for each individual compound corresponds in each case to one line of Table B (compounds I.E.A54.B1 to I.E.A54.B21).

Table 55e

Compounds of the formula I.E in which R¹ corresponds to line A-55 of Table A and the meaning for R² for each individual compound corresponds in each case to one line of Table B (compounds I.E.A55.B1 to I.E.A55.B21).

Table 56e

Compounds of the formula I.E in which R¹ corresponds to line A-56 of Table A and the meaning for R² for each individual compound corresponds in each case to one line of Table B (compounds I.E.A56.B1 to I.E.A56.B21).

Table 57e

Compounds of the formula I.E in which R¹ corresponds to line A-57 of Table A and the meaning for R² for each individual compound corresponds in each case to one line of Table B (compounds I.E.A57.B1 to I.E.A57.B21).

Table 58e

Compounds of the formula I.E in which R¹ corresponds to line A-58 of Table A and the meaning for R² for each individual compound corresponds in each case to one line of Table B (compounds I.E.A58.B1 to I.E.A58.B21).

Table 59e

Compounds of the formula I.E in which R¹ corresponds to line A-59 of Table A and the meaning for R² for each individual compound corresponds in each case to one line of Table B (compounds I.E.A59.B1 to I.E.A59.B21).

Table 60e

Compounds of the formula I.E in which R¹ corresponds to line A-60 of Table A and the meaning for R² for each individual compound corresponds in each case to one line of Table B (compounds I.E.A60.B1 to I.E.A60.B21).

Table 61e

Compounds of the formula I.E in which R¹ corresponds to line A-61 of Table A and the meaning for R² for each individual compound corresponds in each case to one line of Table B (compounds I.E.A61.B1 to I.E.A61.B21).

Table 62e

Compounds of the formula I.E in which R¹ corresponds to line A-62 of Table A and the meaning for R² for each individual compound corresponds in each case to one line of Table B (compounds I.E.A62.B1 to I.E.A62.B21).

Table 63e

Compounds of the formula I.E in which R¹ corresponds to line A-63 of Table A and the meaning for R² for each individual compound corresponds in each case to one line of Table B (compounds I.E.A63.B1 to I.E.A63.B21).

Table 64e

Compounds of the formula I.E in which R¹ corresponds to line A-64 of Table A and the meaning for R² for each individual compound corresponds in each case to one line of Table B (compounds I.E.A64.B1 to I.E.A64.B21).

Table 65e

Compounds of the formula I.E in which R¹ corresponds to line A-65 of Table A and the meaning for R² for each individual compound corresponds in each case to one line of Table B (compounds I.E.A65.B1 to I.E.A65.B21).

Table 66e

Compounds of the formula I.E in which R¹ corresponds to line A-66 of Table A and the meaning for R² for each individual compound corresponds in each case to one line of Table B (compounds I.E.A66.B1 to I.E.A66.B21).

Table 67e

Compounds of the formula I.E in which R¹ corresponds to line A-67 of Table A and the meaning for R² for each individual compound corresponds in each case to one line of Table B (compounds I.E.A67.B1 to I.E.A67.B21).

Table 68e

Compounds of the formula I.E in which R¹ corresponds to line A-68 of Table A and the meaning for R² for each individual compound corresponds in each case to one line of Table B (compounds I.E.A68.B1 to I.E.A68.B21).

Table 69e

Compounds of the formula I.E in which R¹ corresponds to line A-69 of Table A and the meaning for R² for each individual compound corresponds in each case to one line of Table B (compounds I.E.A69.B1 to I.E.A69.B21).

Table 70e

Compounds of the formula I.E in which R¹ corresponds to line A-70 of Table A and the meaning for R² for each individual compound corresponds in each case to one line of Table B (compounds I.E.A70.B1 to I.E.A70.B21).

Table 71e

Compounds of the formula I.E in which R¹ corresponds to line A-71 of Table A and the meaning for R² for each individual compound corresponds in each case to one line of Table B (compounds I.E.A71.B1 to I.E.A71.B21).

Table 72e

Compounds of the formula I.E in which R¹ corresponds to line A-72 of Table A and the meaning for R² for each individual compound corresponds in each case to one line of Table B (compounds I.E.A72.B1 to I.E.A72.B21).

Table 73e

Compounds of the formula I.E in which R¹ corresponds to line A-73 of Table A and the meaning for R² for each individual compound corresponds in each case to one line of Table B (compounds I.E.A73.B1 to I.E.A73.B21).

Table 74e

Compounds of the formula I.E in which R¹ corresponds to line A-74 of Table A and the meaning for R² for each individual compound corresponds in each case to one line of Table B (compounds I.E.A74.B1 to I.E.A74B21).

Table 75e

Compounds of the formula I.E in which R¹ corresponds to line A-75 of Table A and the meaning for R² for each individual compound corresponds in each case to one line of Table B (compounds I.E.A75.B1 to I.E.A75.B21).

Table 76e

Compounds of the formula I.E in which R¹ corresponds to line A-76 of Table A and the meaning for R² for each individual compound corresponds in each case to one line of Table B (compounds I.E.A76.B1 to I.E.A76.B21).

Table 77e

Compounds of the formula I.E in which R¹ corresponds to line A-77 of Table A and the meaning for R² for each individual compound corresponds in each case to one line of Table B (compounds I.E.A77.B1 to I.E.A77.B21).

Table 78e

Compounds of the formula I.E in which R¹ corresponds to line A-78 of Table A and the meaning for R² for each individual compound corresponds in each case to one line of Table B (compounds I.E.A78.B1 to I.E.A78.B21).

Table 79e

Compounds of the formula I.E in which R¹ corresponds to line A-79 of Table A and the meaning for R² for each individual compound corresponds in each case to one line of Table B (compounds I.E.A79.B1 to I.E.A79.B21).

Table 80e

Compounds of the formula I.E in which R¹ corresponds to line A-80 of Table A and the meaning for R² for each individual compound corresponds in each case to one line of Table B (compounds I.E.A80.B1 to I.E.A80.B21).

Table 81e

Compounds of the formula I.E in which R¹ corresponds to line A-81 of Table A and the meaning for R² for each individual compound corresponds in each case to one line of Table B (compounds I.E.A81.B1 to I.E.A81.B21).

Table 82e

Compounds of the formula I.E in which R¹ corresponds to line A-82 of Table A and the meaning for R² for each individual compound corresponds in each case to one line of Table B (compounds I.E.A82.B1 to I.E.A82.B21).

Table 83e

Compounds of the formula I.E in which R¹ corresponds to line A-83 of Table A and the meaning for R² for each individual compound corresponds in each case to one line of Table B (compounds I.E.A83.B1 to I.E.A83.B21).

Table 84e

Compounds of the formula I.E in which R¹ corresponds to line A-84 of Table A and the meaning for R² for each individual compound corresponds in each case to one line of Table B (compounds I.E.A84.B1 to I.E.A84.B21).

Table 85e

Compounds of the formula I.E in which R¹ corresponds to line A-85 of Table A and the meaning for R² for each individual compound corresponds in each case to one line of Table B (compounds I.E.A85.B1 to I.E.A85.B21).

Table 86e

Compounds of the formula I.E in which R¹ corresponds to line A-86 of Table A and the meaning for R² for each individual compound corresponds in each case to one line of Table B (compounds I.E.A86.B1 to I.E.A86.B21).

Table 87e

Compounds of the formula I.E in which R¹ corresponds to line A-87 of Table A and the meaning for R² for each individual compound corresponds in each case to one line of Table B (compounds I.E.A87.B1 to I.E.A87.B21).

Table 88e

Compounds of the formula I.E in which R¹ corresponds to line A-88 of Table A and the meaning for R² for each individual compound corresponds in each case to one line of Table B (compounds I.E.A88.B1 to I.E.A88.B21).

Table 89e

Compounds of the formula I.E in which R¹ corresponds to line A-89 of Table A and the meaning for R² for each individual compound corresponds in each case to one line of Table B (compounds I.E.A89.B1 to I.E.A89.B21).

Table 90e

Compounds of the formula I.E in which R¹ corresponds to line A-90 of Table A and the meaning for R² for each individual compound corresponds in each case to one line of Table B (compounds I.E.A90.B1 to I.E.A90.B21).

Table 91e

Compounds of the formula I.E in which R¹ corresponds to line A-91 of Table A and the meaning for R² for each individual compound corresponds in each case to one line of Table B (compounds I.E.A91.B1 to I.E.A91.B21).

Table 92e

Compounds of the formula I.E in which R¹ corresponds to line A-92 of Table A and the meaning for R² for each individual compound corresponds in each case to one line of Table B (compounds I.E.A92.B1 to I.E.A92.B21).

Table 93e

Compounds of the formula I.E in which R¹ corresponds to line A-93 of Table A and the meaning for R² for each individual compound corresponds in each case to one line of Table B (compounds I.E.A93.B1 to I.E.A93.B21).

Table 94e

Compounds of the formula I.E in which R¹ corresponds to line A-94 of Table A and the meaning for R² for each individual compound corresponds in each case to one line of Table B (compounds I.E.A94.B1 to I.E.A94.B21).

Table 95e

Compounds of the formula I.E in which R¹ corresponds to line A-95 of Table A and the meaning for R² for each individual compound corresponds in each case to one line of Table B (compounds I.E.A95.B1 to I.E.A95.B21).

Table 96e

Compounds of the formula I.E in which R¹ corresponds to line A-96 of Table A and the meaning for R² for each individual compound corresponds in each case to one line of Table B (compounds I.E.A96.B1 to I.E.A96.B21).

Table 97e

Compounds of the formula I.E in which R¹ corresponds to line A-97 of Table A and the meaning for R² for each individual compound corresponds in each case to one line of Table B (compounds I.E.A97.B1 to I.E.A97.B21).

Table 98e

Compounds of the formula I.E in which R¹ corresponds to line A-98 of Table A and the meaning for R² for each individual compound corresponds in each case to one line of Table B (compounds I.E.A98.B1 to I.E.A98.B21).

Table 99e

Compounds of the formula I.E in which R¹ corresponds to line A-99 of Table A and the meaning for R² for each individual compound corresponds in each case to one line of Table B (compounds I.E.A99.B1 to I.E.A99.B21).

Table 100e

Compounds of the formula I.E in which R¹ corresponds to line A-100 of Table A and the meaning for R² for each individual compound corresponds in each case to one line of Table B (compounds I.E.A100.B1 to I.E.A100.B21).

Table 101e

Compounds of the formula I.E in which R¹ corresponds to line A-101 of Table A and the meaning for R² for each individual compound corresponds in each case to one line of Table B (compounds I.E.A101.B1 to I.E.A101.B21).

TABLE A

| | R¹ |
|---|---|
| A-1 | H |
| A-2 | —CH₃ |
| A-3 | —CH₂CH₃ |
| A-4 | —CH₂CH₂CH₃ |
| A-5 | —CH(CH₃)₂ |
| A-6 | —CH₂CH₂CH₂CH₃ |
| A-7 | —CH(CH₂CH₃)₂ |
| A-8 | —C(CH₃)₃ |
| A-9 | —CH₂CH(CH₃)₂ |
| A-10 | —CH₂CH₂CH₂CH₂CH₃ |
| A-11 | —CH₂CH₂CH₂CH₂CH₂CH₃ |
| A-12 | CF₃ |
| A-13 | CHF₂ |
| A-14 | —CH═CH₂ |
| A-15 | —CH═CHCH₃ |
| A-16 | —CH₂CH═CH₂ |
| A-17 | —C≡CH |
| A-18 | —C≡CCH₃ |
| A-19 | —CH₂C≡CH |
| A-20 | —C₃H₅ (cyclopropyl) |
| A-21 | —C₅H₉ (cyclopentyl) |
| A-22 | phenyl |
| A-23 | benzyl |
| A-24 | 4-Cl-phenyl |
| A-25 | 2,4-Cl₂-phenyl |
| A-26 | OH |
| A-27 | OCH₃ |
| A-28 | OCH₂CH₃ |
| A-29 | OCH₂CH₂CH₃ |
| A-30 | OCH(CH₃)₂ |
| A-31 | OCH₂CH₂CH₂CH₃ |
| A-32 | OCH(CH₂CH₃)₂ |
| A-33 | OC(CH₃)₃ |
| A-34 | OCH₂CH(CH₃)₂ |
| A-35 | OCH₂CH₂CH₂CH₂CH₃ |
| A-36 | OCH₂CH₂CH₂CH₂CH₂CH₃ |
| A-37 | NMe₂ |
| A-38 | NEt₂ |
| A-39 | CH₂CF₃ |
| A-40 | CH(CH₃)CH₂CH₃ |
| A-41 | CH₂CH₂CH═CH₂ |
| A-42 | CH(CH₃)CH(OCH₃)₂ |
| A-43 | NH₂ |
| A-44 | NHMe |
| A-45 | NHEt |
| A-46 | CH₂OH |
| A-47 | CH₂Cl |
| A-48 | CH₂Br |
| A-49 | CH₂OMe |
| A-50 | CH₂OEt |
| A-51 | CH₂OCH₂CH₂CH₃ |
| A-52 | CH₂OCH(CH₃)₂ |
| A-53 | CH₂OCH₂CH₂CH₂CH₃ |
| A-54 | CH₂ |
| A-55 | CH₂CN |
| A-56 | CH₂CH₂CN |
| A-57 | CH₂OCHF₂ |
| A-58 | CH₂OCF₃ |
| A-59 | CH₂COOH |
| A-60 | CH₂COOMe |
| A-61 | CH₂COOEt |
| A-62 | CH₂CONH₂ |
| A-63 | CH₂CONHMe |
| A-64 | CH₂CONHEt |
| A-65 | CH₂CON(Me)₂ |
| A-66 | CH₂CON(Et)₂ |
| A-67 | CH(CH₃)CN |
| A-68 | CH(CH₃)CH₂CN |
| A-69 | CH(CH₃)OCHF₂ |
| A-70 | CH(CH₃)OCF₃ |
| A-71 | CH(CH₃)COOH |
| A-72 | CH(CH₃)COOMe |
| A-73 | CH(CH₃)COOEt |
| A-74 | CH(CH₃)CONH₂ |
| A-75 | CH(CH₃)CONHMe |
| A-76 | CH(CH₃)CONHEt |
| A-77 | CH(CH₃)CON(Me)₂ |
| A-78 | CH(CH₃)CON(Et)₂ |
| A-79 | C(CH₃)₂CN |
| A-80 | C(CH₃)₂CH₂CN |
| A-81 | C(CH₃)₂OCHF₂ |
| A-82 | C(CH₃)₂OCF₃ |
| A-83 | C(CH₃)₂COOH |
| A-84 | C(CH₃)₂COOMe |
| A-85 | C(CH₃)₂COOEt |
| A-86 | C(CH₃)₂CONH₂ |
| A-87 | C(CH₃)₂CONHMe |
| A-88 | C(CH₃)₂CONHEt |
| A-89 | C(CH₃)₂CON(Me)₂ |
| A-90 | C(CH₃)₂CON(Et)₂ |
| A-91 | CHO |
| A-92 | COOH |
| A-93 | COOMe |
| A-94 | COOEt |
| A-95 | CONH₂ |
| A-96 | CONHMe |
| A-97 | CONHEt |
| A-98 | CON(Me)₂ |
| A-99 | CON(Et)₂ |
| A-100 | CN |
| A-101 | cyclohexyl |

TABLE B

| | R² |
|---|---|
| B-1 | CN |
| B-2 | NO₂ |
| B-3 | —C(═O)CH₃ |
| B-4 | —C(═O)CH₂CH₃ |
| B-5 | —C(═O)CH(CH₃)₂ |
| B-6 | —C(═O)C(CH₃)₃ |
| B-7 | —C(═O)OH |
| B-8 | —C(═O)OCH₃ |
| B-9 | —C(═O)OCH₂CH₃ |
| B-10 | —C(═O)OCH(CH₃)₂ |
| B-11 | —C(═O)OC(CH₃)₃ |
| B-12 | —C(═O)NH₂ |
| B-13 | —C(═O)NHCH₃ |
| B-14 | —C(═O)NHCH₂CH₃ |
| B-15 | —C(═O)NHCH(CH₃)₂ |
| B-16 | —C(═O)NHC(CH₃)₃ |
| B-17 | —C(═O)N(CH₃)₂ |
| B-18 | —C(═O)N(CH₂CH₃)₂ |
| B-19 | —C(═O)N(CH(CH₃)₂)₂ |
| B-20 | —C(═O)N(C(CH₃)₃)₂ |
| B-21 | H |

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as 5-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enzyme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The compounds I and compositions thereof, respectively, are particularly suitable for controlling the following plant diseases:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochllobolus spp.), e. g. Southern leaf blight (D. maydis) or Northern leaf blight (B. zeicola) on corn, e. g. spot blotch (B. sorokiniana) on cereals and e.g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e.g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochllobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporiodes); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria lirodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophllum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilium (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans and F. verticilliodes on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotima spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici(Septoria blotch) on wheat and S. (syn. Stagonospora) nodorum (Stagonospora blotch) on cereals; Uncinula (syn. Erysiphe) necator (powdery mildew, anamorph: Odium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

The compounds I and compositions thereof, respectively, are also suitable for controlling harmful fungi in the protection of stored products or harvest and in the protection of materials. The term “protection of materials” is to be understood to denote the protection of technical and nonliving materials, such as adhesives, glues, wood, paper and paperboard, textiles, leather, paint dispersions, plastics, coiling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichorma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and compositions thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and compositions thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and compositions thereof.

Suitable surfactants are surface-active compounds, such as anionic, cationic, nonionic and amphoteric surfactants, block polymers, polyelectrolytes, and compositions thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and compositions thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and compositions thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound I and 5-15 wt % wetting agent (e.g. alcohol alkoxylates) are dissolved in water and/or in a water-soluble solvent (e.g. alcohols) ad 100 wt %. The active substance dissolves upon dilution with water.

### ii) Dispersible Concentrates (DC)

5-25 wt % of a compound I and 1-10 wt % dispersant (e. g. polyvinylpyrrolidone) are dissolved in organic solvent (e.g. cyclohexanone) ad 100 wt %. Dilution with water gives a dispersion.

iii) Emulsifiable Concentrates (EC)

15-70 wt % of a compound I and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

### iv) Emulsions (EW, EO, ES)

5-40 wt % of a compound I and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This composition is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a compound I are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

### vi) Water-Dispersible Granules and Water-Soluble Granules (WG, SG)

50-80 wt % of a compound I are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

### ix) Microemulsion (ME)

5-20 wt % of a compound I are added to 5-30 wt % organic solvent blend (e.g. fatty acid dimethylamide and cyclohexanone), 10-25 wt % surfactant blend (e.g. alcohol ethoxylate and arylphenol ethoxylate), and water ad 100%. This composition is stirred for 1 h to produce spontaneously a thermodynamically stable microemulsion.

### x) Microcapsules (CS)

An oil phase comprising 5-50 wt % of a compound I, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS composition.

xi) Dustable powders (DP, DS)

1-10 wt % of a compound I are ground finely and mixed intimately with solid carrier (e.g. finely divided kaolin) ad 100 wt %.

xii) Granules (GR, FG)

0.5-30 wt % of a compound I is ground finely and associated with solid carrier (e.g. silicate) ad 100 wt %. Granulation is achieved by extrusion, spray-drying or fluidized bed.

xiii) Ultra-Low Volume Liquids (UL)

1-50 wt % of a compound I are dissolved in organic solvent (e.g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary composition may be mixed by the user himself in a spray tank and further auxiliaries may be added, if appropriate.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of active substances, in conjunction with which the compounds I can be used, is intended to illustrate the possible combinations but does not limit them:

A) Respiration inhibitors
Inhibitors of complex III at Qₒ site (e.g. strobilurins): azoxystrobin, coumethoxystrobin, coumoxystrobin, dimoxystrobin, enestroburin, fenaminstrobin, fenoxystrobin/flufenoxystrobin, fluoxastrobin, kresoxim-methyl, metominostrobin, orysastrobin, picoxystrobin, pyraclostrobin, pyrametostrobin, pyraoxystrobin, trifloxystrobin, 2-[2-(2,5-dimethyl-phenoxymethyl)-phenyl]-3-methoxy-acrylic acid methyl ester and 2-(2-(3-(2,6-dichlorophenyl)-1-methyl-allylideneaminooxymethyl)-phenyl)-2-methoxyimino-N-methyl-acetamide, pyribencarb, triclopyricarb/chlorodincarb, famoxadone, fenamidone;
inhibitors of complex III at Qᵢ site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl] 2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl] 2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl] 2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl] 2-methylpropanoate; (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate
   inhibitors of complex II (e. g. carboxamides): benodanil, benzovindiflupyr, bixafen, boscalid, carboxin, fenfuram, fluopyram, flutolanil, fluxapyroxad, furametpyr, isofetamid, isopyrazam, mepronil, oxycarboxin, penflufen, penthiopyrad, sedaxane, tecloftalam, thifluzamide, N-(4′-trifluoromethylthiobiphenyl-2-yl)-3-difluoromethyl-1-methyl-1H-pyrazole-4-carboxamide, N-(2-(1,3,3-trimethyl-butyl)-phenyl)-1,3-dimethyl-5-fluoro-1H-pyrazole-4-carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide; N-(7-fluoro-1,1,3-trimethyl-indan-4-yl)-1,3-dimethyl-pyrazole-4-carboxamide, N-[2-(2,4-dichlorophenyl)-2-methoxy-1-methyl-ethyl]-3-(difluoromethyl)-1-methyl-pyrazole-4-carboxamide
   N-[2-(2,4-dichlorophenyl)-2-methoxy-1-methyl-ethyl]-3-(difluoromethyl)-1-methyl-pyrazole-4-carboxamide
other respiration inhibitors (e.g. complex I, uncouplers): diflumetorim, (5,8-difluoroquinazolin-4-yl)-{2-[2-fluoro-4-(4-trifluoromethylpyridin-2-yloxy)-phenyl]-ethyl}-amine; nitrophenyl derivates: binapacryl, dinobuton, dinocap, fluazinam; ferimzone; organometal compounds: fentin salts, such as fentin-acetate, fentin chloride or fentin hydroxide; ametoctradin; and silthiofam;

B) Sterol biosynthesis inhibitors (SBI fungicides)
C14 demethylase inhibitors (DMI fungicides): triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole,
1-[rel-(2S,3R)-3-(2-chlorophenyl)-2-(2,4-difluorophenyl)-oxiranylmethyl]-5-thiocyanato-1H-[1,2,4]triazole, 2-[rel-(2S,3R)-3-(2-chlorophenyl)-2-(2,4-difluorophenyl)-oxiranylmethyl]-2H-[1,2,4]triazole-3-thiol; 2-[2-chloro-4-(4-chlorophenoxyl)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxyl)phenyl]-1-(1,2,4-triazol-1-yl)butan-2-ol (B.1.36), 2-[4-(4-chlorophenoxy)-2-(trifluoromethyl)phenyl]-3-methyl-1-(1,2,4-triazol-1-yl)butan-2-ol (B.1.37), 2-[4-(4-chlorophenoxy)-2-(trifluoromethyl)phenyl]-1-(1,2,4-triazol-1-yl)propan-2-ol (B.1.38), 2-[2-chloro-4-(4-chlorophenoxyl)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol; imidazoles: imazalil, pefurazoate, prochloraz, triflumizol; pyrimidines, pyridines and piperazines: fenarimol, nuarimol, pyrifenox, triforine), [3-(4-chloro-2-fluoro-phenyl)-5-(2,4-difluorophenyl)isoxazol-4-yl]-(3-pyridyl)methanol;
Delta 14-reductase inhibitors: aldimorph, dodemorph, dodemorph-acetate, fenpropimorph, tridemorph, fenpropidin, piperalin, spiroxamine;
Inhibitors of 3-keto reductase: fenhexamid;

C) Nucleic acid synthesis inhibitors
phenylamides or acyl amino acid fungicides: benalaxyl, benalaxyl-M, kiralaxyl, metalaxyl, metalaxyl-M (mefenoxam), ofurace, oxadixyl;
others: hymexazole, octhilinone, oxolinic acid, bupirimate, 5-fluorocytosine, 5-fluoro-2-(p-tolylmethoxy)pyrimidin-4-amine, 5-fluoro-2-(4-fluorophenylmethoxyl)pyrimidin-4-amine;

D) Inhibitors of cell division and cytoskeleton
tubulin inhibitors, such as benzimidazoles, thiophanates: benomyl, carbendazim, fuberidazole, thiabendazole, thiophanate-methyl; triazolopyrimidines: 5-chloro-7-(4-methyl-piperidin-1-yl)-6-(2,4,6-trifluorophenyl)-[1,2,4]triazolo[1,5-a]pyrimidine
other cell division inhibitors: diethofencarb, ethaboxam, pencycuron, fluopicolide, zoxamide, metrafenone, pyriofenone;

E) Inhibitors of amino acid and protein synthesis
methionine synthesis inhibitors (anilino-pyrimidines): cyprodinil, mepanipyrim, pyrimethanil;
protein synthesis inhibitors: blasticidin-S, kasugamycin, kasugamycin hydrochloride-hydrate, mildiomycin, streptomycin, oxytetracyclin, polyoxine, validamycin A;

F) Signal transduction inhibitors
MAP/histidine kinase inhibitors: fluoroimid, iprodione, procymidone, vinclozolin, fenpiclonil, fludioxonil;
G protein inhibitors: quinoxyfen;

G) Lipid and membrane synthesis inhibitors
Phospholipid biosynthesis inhibitors: edifenphos, iprobenfos, pyrazophos, isoprothiolane;
lipid peroxidation: dicloran, quintozene, tecnazene, tolclofos-methyl, biphenyl, chloroneb, etridiazole;
phospholipid biosynthesis and cell wall deposition: dimethomorph, flumorph, mandipropamid, pyrimorph, benthiavalicarb, iprovalicarb, valifenalate and N-(1-(1-(4-cyano-phenyl)-ethanesulfonyl)-but-2-yl) carbamic acid-(4-fluorophenyl) ester;
compounds affecting cell membrane permeability and fatty acids: propamocarb, —fatty acid amide hydrolase inhibitors: oxathiapiprolin, 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate, 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate; H) Inhibitors with Multi Site Action
inorganic active substances: Bordeaux composition, copper acetate, copper hydroxide, copper oxychloride, basic copper sulfate, sulfur;
thio- and dithiocarbamates: ferbam, mancozeb, maneb, metam, metiram, propineb, thiram, zineb, ziram;
organochlorine compounds (e.g. phthalimides, sulfamides, chloronitriles): anilazine, chlorothalonil, captafol, captan, folpet, dichlofluanid, dichlorophen, hexachlorobenzene, pentachlorphenole and its salts, phthalide, tolylfluanid, N-(4-chloro-2-nitro-phenyl)-N-ethyl-4-methyl-benzenesulfonamide;
guanidines and others: guanidine, dodine, dodine free base, guazatine, guazatine-acetate, iminoctadine, iminoctadine-triacetate, iminoctadine-tris(albesilate), dithianon, 2,6-dimethyl-1H,5H-[1,4]dithiino[2,3-c:5,6-c′]dipyrrole-1,3,5,7(2H,6H)-tetraone;

I) Cell wall synthesis inhibitors
inhibitors of glucan synthesis: validamycin, polyoxin B; melanin synthesis inhibitors: pyroquilon, tricyclazole, carpropamid, dicyclomet, fenoxanil;

J) Plant defence inducers
acibenzolar-S-methyl, probenazole, isotianil, tiadinil, prohexadione-calcium; phosphonates: fosetyl, fosetyl-aluminum, phosphorous acid and its salts;

K) Unknown mode of acti-bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothal-isopropyl, oxathiapiprolin, oxin-copper, proquinazid, tebufloquin, tecloftalam, tolprocarb, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2-methyl-4-(3-trimethylsilanyl-propoxy)-phenyl)-N-ethyl-N-methyl formamidine, methoxy-acetic acid 6-tert-butyl-8-fluoro-2,3-dimethyl-quinolin-4-yl ester, 3-[5-(4-methylphenyl)-2,3-dimethyl-isoxazolidin-3-yl]-pyridine, 3-[5-(4-chloro-phenyl)-2,3-dimethyl-isoxazolidin-3-yl]-pyridine (pyrisoxazole), N-(6-methoxy-pyridin-3-yl)cyclopropanecarboxylic acid amide, 5-chloro-1-(4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide; and 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone,

2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, ethyl(Z)-3-amino-2-cyano-3-phenyl-prop-2-enoate, picarbutrazox, pentyl N-[6-[[(Z)-[(1-methyltetrazol-5-yl)-phenyl-methylene]amino]oxymethyl]-2-pyridyl]carbamate (K.1.42), 2-[2-[(7,8-difluoro-2-methyl-3-quinolyl)oxy]-6-fluoro-phenyl]propan-2-ol, 2-[2-fluoro-6-[(8-fluoro-2-methyl-3-quinolyl)oxy]phenyl]propan-2-ol, 3-(5-fluoro-3,3,4,4-tetramethyl-3,4-dihydroisoquinolin-1-yl)quinoline, 3-(4,4-difluoro-3,3-dimethyl-3,4-dihydroisoquinolin-1-yl)quinoline, 3-(4,4,5-trifluoro-3,3-dimethyl-3,4-dihydroisoquinolin-1-yl)quinoline;

L) L) Biopesticides
L1) Microbial pesticides with fungicidal, bactericidal, viricidal and/or plant defense activator activity: Ampelomyces quisqualis, Aspergillus flavus, Aureobasidium pullulans, Bacillus amyloliquefaciens, B. mojavensis, B. pumilus, B. simplex, B. solisalsi, B. subtilis, B. subtilis var. amyloliquefaciens, Candida oleophila, C. saitoana, Clavibacter michiganensis (bacteriophages), Coniothyrium minitans, Cryphonectria parasitica, Cryptococcus albidus, Dilophosphora alopecuri, Fusarium oxysporum, Clonostachys rosea f. catenulate (also named Gliocladium catenulatum), Gliocladium roseum, Lysobacter antibioticus, L. enzymogenes, Metschnikowia fructicola, Microdochium dimerum, Microsphaeropsis ochracea, Muscodor albus, Paenibacillus polymyxa, Pantoea vagans, Phlebiopsis gigantea, Pseudomonas sp., Pseudomonas chloraphis, Pseudozyma flocculosa, Pichia anomala, Pythium oligandrum, Sphaerodes mycoparasitica, Streptomyces griseoviridis, S. lydicus, S. violaceusniger, Talaromyces flavus, Trichoderma asperellum, T. atroviride, T. fertile, T. gamsii, T. harmatum, T. harzianum; mixture of T. harzianum and T. viride; mixture of T. polysporum and T. harzianum; T. stromaticum, T. virens (also named Gliocladium virens), T. viride, Typhula phacorrhiza, Ulocladium oudemansii, Verticillium dahlia, zucchini yellow mosaic virus (avirulent strain);
L2) Biochemical pesticides with fungicidal, bactericidal, viricidal and/or plant defense activator activity: chitosan (hydrolysate), harpin protein, laminarin, Menhaden fish oil, natamycin, Plum pox virus coat protein, potassium or sodium bicarbonate, Reynoutria sachlinensis extract, salicylic acid, tea tree oil;
L3) Microbial pesticides with insecticidal, acaricidal, molluscidal and/or nematicidal activity: Agrobacterium radiobacter, Bacillus cereus, B. firmus, B. thuringiensis, B. thuringiensis ssp. aizawai, B. t. ssp. israelensis, B. t. ssp. galleriae, B. t. ssp. kurstaki, B. t. ssp. tenebrionis, Beauveria bassiana, B. brongniartii, Burkholderia sp., Chromobacterium subtsugae, Cydia pomonella granulosis virus, Cryptophlebia leucotreta granulovirus (CrleGV), Isaria fumosorosea, Heterorhabditis bacteriophora, Lecanicillium longisporum, L. muscarium (formerly Verticillium lecanii), Metarhizium anisopliae, M. anisopliae var. acridum, Nomuraea rileyi, Paecilomyces fumosoroseus, P. lilacinus, Paenibacillus popilliae, Pasteuria spp., P. nishizawae, P. penetrans, P. ramose, P. reneformis, P. thornea, P. usgae, Pseudomonas fluorescens, Steinernema carpocapsae, S. feltiae, S. kraussei;
L4) Biochemical pesticides with insecticidal, acaricidal, molluscidal, pheromone and/or nematicidal activity: L-carvone, citral, (E,Z)-7,9-dodecadien-1-ylacetate, ethyl formate, (E,Z)-2,4-ethyl decadienoate (pear ester), (Z,Z,E)-7,11,13-hexadecatrienal, heptyl butyrate, isopropyl myristate, lavanulyl senecioate, cis-jasmone, 2-methyl 1-butanol, methyl eugenol, methyl jasmonate, (E,Z)-2,13-octadecadien-1-ol, (E,Z)-2,13-octadecadien-1-ol acetate, (E,Z)-3,13-octadecadien-1-ol, R-1-octen-3-ol, pentatermanone, potassium silicate, sorbitol actanoate, (E,Z,Z)-3,8,11-tetradecatrienyl acetate, (Z,E)-9,12-tetradecadien-1-ylacetate, Z-7-tetradecen-2-one, Z-9-tetradecen-1-ylacetate, Z-11-tetradecenal, Z-11-tetradecen-1-ol, Acacia negra extract, extract of grapefruit seeds and pulp, extract of Chenopodium ambrosiodae, Catnip oil, Neem oil, Quillay extract, Tagetes oil;
L5) Microbial pesticides with plant stress reducing, plant growth regulator, plant growth promoting and/or yield enhancing activity: Azospirillum amazonense, A. brasilense, A. lipoferum, A. irakense, A. halopraeferens, Bradyrhizobium sp., B. elkanii, B. japonicum, B. liaoningense, B. lupini, Delftia acidovorans, Glomus intraradices, Mesorhizobium sp., Paenibacillus alvei, Penicillium bilaiae, Rhizobium leguminosarum bv. phaseoli RG-B10 (L.5.54), R. l. bv. trifolii RP113-7 (L.5.55), R. l. bv. trifolii 095 (L.5.63), R. l. bv. trifolii TA1 (L.5.64), R. l. bv. trifolii CC283b (L.5.65), R. l. bv. trifolii CC275e (L.5.66), R. l. bv. trifolii CB782 (L.5.67), R. l. bv. trifolii CC1099 (L.5.68), R. l. bv. trifolii WSM1325 (L.5.69), R. l. bv. viciae SU303 (L.5.56), R. l. bv. viciae WSM1455 (L.5.57), R. l. bv. viciae P1NP3Cst (L.5.58) R. l. bv. viciae RG-P2 (L.5.70), R. tropici SEMIA 4080 (L.5.59), R. tropici SEMIA 4077 (L.5.71), R. tropici CC511 (L.5.72), Sinorhizobium meliloti MSDJ0848 (L.5.60), S. meliloti NRG185 (L.5.61), S. meliloti RR1128, (L.5.62); R. l. trifolii, R. l. bv. viciae, R. tropici, Sinorhizobium meliloti;
L6) Biochemical pesticides with plant stress reducing, plant growth regulator and/or plant yield enhancing activity: abscisic acid, aluminium silicate (kaolin), 3-decen-2-one, formononetin, genistein, hesperetin, homobrassinlide, humates, jasmonic acid or salts or derivatives thereof, lysophosphatidyl ethanolamine, naringenin, polymeric polyhydroxy acid, Ascophyllum nodosum (Norwegian kelp, Brown kelp) extract and Ecklonia maxima (kelp) extract;

M) Growth regulators

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole;

N) Herbicides
acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid derivatives: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
Bipyridyls: diquat, paraquat;
(thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bomoxynil, dichlobenil, ioxynil;
imidazolinones: imazamethabenz, imazamox, imazapic, imazapyr, imazaquin, imazethapyr;
phenoxy acetic acids: clomeprop, 2,4-dichlorophenoxyacetic acid (2,4-D), 2,4-DB, dichlorprop, MCPA, MCPA-thioethyl, MCPB, Mecoprop;
pyrazines: chloridazon, flufenpyr-ethyl, fluthiacet, norflurazon, pyridate;
pyridines: aminopyralid, clopyralid, diflufenican, dithiopyr, fluridone, fluroxypyr, picloram, picolinafen, thiazopyr;
sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metazosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxy-pyrimidin-2-yl)urea;
triazines: ametryn, atrazine, cyanazine, dimethametryn, ethiozin, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bicyclopyrone, bromacil, bromobutide, butafenacil, butamifos, cafenstrole, carfentrazone, cinidon-ethyl, chlorthal, cinmethylin, clomazone, cumyluron, cyprosulfamide, dicamba, difenzoquat, diflufenzopyr, Drechslera monoceras, endothal, ethofumesate, etobenzanid, fenoxasulfone, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

O) Insecticides
organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphosmethyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alphacypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
insect growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
nicotinic receptor agonists/antagonists compounds: clothianidin, dinotefuran, flupyradifurone, imidacloprid, thiamethoxam, nitenpyram, acetamiprid, thiacloprid, 1-2-chloro-thiazol-5-ylmethyl)-2-nitrimino-3,5-dimethyl-[1,3,5]triazinane;
GABA antagonist compounds: endosulfan, ethiprole, fipronil, vaniliprole, pyrafluprole, pyriprole, 5-amino-1-(2,6-dichloro-4-methyl-phenyl)-4-sulfinamoyl-1H-pyrazole-3-carbothioic acid amide;
macrocyclic lactone insecticides: abamectin, emamectin, milbemectin, lepimectin, spinosad, spinetoram;
mitochondrial electron transport inhibitor (METI) I acaricides: fenazaquin, pyridaben, tebufenpyrad, tolfenpyrad, flufenerim;
METI II and III compounds: acequinocyl, fluacyprim, hydramethylnon;
Uncouplers: chlorfenapyr;
oxidative phosphorylation inhibitors: cyhexatin, diafenthiuron, fenbutatin oxide, propargite;
moulting disruptor compounds: cryomazine;
mixed function oxidase inhibitors: piperonyl butoxide;
sodium channel blockers: indoxacarb, metaflumizone;
ryanodine receptor inhibitors: chlorantraniliprole, cyantraniliprole, flubendiamide, N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide; N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide; N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide; N-[4,6-dichloro-2-[(diethyl-lambda-4-sulfanylidene)carbamoyl]-phenyl]-2-(3-chloro-2-pyridyl)-5-(difluoromethyl)pyrazole-3-carboxamide; N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide; N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide; N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide;
   others: benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluron, pyrifluquinazon and 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]cyclopropaneacetic acid ester.

The present invention furthermore relates to agrochemical compositions comprising a composition of at least one compound I (component 1) and at least one further active substance useful for plant protection, e. g. selected from the groups A) to O) (component 2), in particular one further fungicide, e. g. one or more fungicide from the groups A) to L), as described above, and if desired one suitable solvent or solid carrier. Those compositions are of particular interest, since many of them at the same application rate show higher efficiencies against harmful fungi. Furthermore, combating harmful fungi with a composition of compounds I and at least one fungicide from groups A) to L), as described above, is more efficient than combating those fungi with individual compounds I or individual fungicides from groups A) to L). By applying compounds I together with at least one active substance from groups A) to O) a synergistic effect can be obtained, i.e. more then simple addition of the individual effects is obtained (synergistic compositions).

This can be obtained by applying the compounds I and at least one further active substance simultaneously, either jointly (e. g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

In binary compositions, i.e. compositions according to the invention comprising one compound I (component 1) and one further active substance (component 2), e. g. one active substance from groups A) to O), the weight ratio of component 1 and component 2 generally depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:3 to 3:1.

In ternary compositions, i.e. compositions according to the invention comprising one compound I (component 1) and a first further active substance (component 2) and a second further active substance (component 3), e. g. two active substances from groups A) to O), the weight ratio of component 1 and component 2 depends from the properties of the active substances used, preferably it is in the range of from 1:50 to 50:1 and particularly in the range of from 1:10 to 10:1, and the weight ratio of component 1 and component 3 preferably is in the range of from 1:50 to 50:1 and particularly in the range of from 1:10 to 10:1.

Preference is also given to compositions comprising a compound I (component 1) and at least one active substance selected from group A) (component 2) and particularly selected from azoxystrobin, dimoxystrobin, fluoxastrobin, kresoxim-methyl, orysastrobin, picoxystrobin, pyraclostrobin, trifloxystrobin; famoxadone, fenamidone; benzovindiflupyr, bixafen, boscalid, fluopyram, fluxapyroxad, isopyrazam, penflufen, penthiopyrad, sedaxane; ametoctradin, cyazofamid, fluazinam, fentin salts, such as fentin acetate.

Preference is given to compositions comprising a compound of formula I (component 1) and at least one active substance selected from group B) (component 2) and particularly selected from cyproconazole, difenoconazole, epoxiconazole, fluquinconazole, flusilazole, flutriafol, metconazole, myclobutanil, penconazole, propiconazole, prothioconazole, triadimefon, triadimenol, tebuconazole, tetraconazole, triticonazole, prochloraz, fenarimol, triforine; dodemorph, fenpropimorph, tridemorph, fenpropidin, spiroxamine; fenhexamid.

Preference is given to compositions comprising a compound of formula I (component 1) and at least one active substance selected from group C) (component 2) and particularly selected from metalaxyl, (metalaxyl-M) mefenoxam, ofurace.

Preference is given to compositions comprising a compound of formula I (component 1) and at least one active substance selected from group D) (component 2) and particularly selected from benomyl, carbendazim, thiophanate-methyl, ethaboxam, fluopicolide, zoxamide, metrafenone, pyriofenone.

Preference is also given to compositions comprising a compound I (component 1) and at least one active substance selected from group E) (component 2) and particularly selected from cyprodinil, mepanipyrim, pyrimethanil.

Preference is also given to compositions comprising a compound I (component 1) and at least one active substance selected from group F) (component 2) and particularly selected from iprodione, fludioxonil, vinclozolin, quinoxyfen.

Preference is also given to compositions comprising a compound I (component 1) and at least one active substance selected from group G) (component 2) and particularly selected from dimethomorph, flumorph, iprovalicarb, benthiavalicarb, mandipropamid, propamocarb.

Preference is also given to compositions comprising a compound I (component 1) and at least one active substance selected from group H) (component 2) and particularly selected from copper acetate, copper hydroxide, copper oxychloride, copper sulfate, sulfur, mancozeb, metiram, propineb, thiram, captafol, folpet, chlorothalonil, dichlofluanid, dithianon.

Preference is also given to compositions comprising a compound I (component 1) and at least one active substance selected from group I) (component 2) and particularly selected from carpropamid and fenoxanil.

Preference is also given to compositions comprising a compound I (component 1) and at least one active substance selected from group J) (component 2) and particularly selected from acibenzolar-S-methyl, probenazole, tiadinil, fosetyl, fosetyl-aluminium, H₃PO₃ and salts thereof.

Preference is also given to compositions comprising a compound I (component 1) and at least one active substance selected from group K) (component 2) and particularly selected from cymoxanil, proquinazid and N-methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide.

Preference is also given to compositions comprising a compound I (component 1) and at least one active substance selected from group L) (component 2) and particularly selected from Bacillus subtilis strain NRRL No. B-21661, Bacillus pumilus strain NRRL No. B-30087 and Ulocladium oudemansii.

Accordingly, the present invention furthermore relates to compositions comprising one compound I (component 1) and one further active substance (component 2), which further active substance is selected from the column “Component 2” of the lines B-1 to B-369 of Table C.

A further embodiment relates to the compositions B-1 to B-369 listed in Table C, where a row of Table B corresponds in each case to a fungicidal composition comprising one of the in the present specification individualized compounds of formula I (component 1) and the respective further active substance from groups A) to O) (component 2) stated in the row in question. Preferably, the compositions described comprise the active substances in synergistically effective amounts.

TABLE C
Composition comprising one indivivalized compound I and one
further active substance from groups A) to O)

| Compo- | | |
|---|---|---|
| sition | Component 1 | Component 2 |
| C-1 | one individualized compound I | Azoxystrobin |
| C-2 | one individualized compound I | Coumethoxystrobin |
| C-3 | one individualized compound I | Coumoxystrobin |
| C-4 | one individualized compound I | Dimoxystrobin |
| C-5 | one individualized compound I | Enestroburin |
| C-6 | one individualized compound I | Fenaminstrobin |
| C-7 | one individualized compound I | Fenoxystrobin/ |
| | | Flufenoxystrobin |
| C-8 | one individualized compound I | Fluoxastrobin |
| C-9 | one individualized compound I | Kresoxim-methyl |
| C-10 | one individualized compound I | Metominostrobin |
| C-11 | one individualized compound I | Orysastrobin |
| C-12 | one individualized compound I | Picoxystrobin |
| C-13 | one individualized compound I | Pyraclostrobin |
| C-14 | one individualized compound I | Pyrametostrobin |
| C-15 | one individualized compound I | Pyraoxystrobin |
| C-16 | one individualized compound I | Pyribencarb |
| C-17 | one individualized compound I | Trifloxystrobin |
| C-18 | one individualized compound I | Triclopyricarb/ |
| | | Chlorodincarb |
| C-19 | one individualized compound I | 2-[2-(2,5-dimethyl- |
| | | phenoxymethyl)-phenyl]-3- |
| | | methoxy-acrylic acid methyl |
| | | ester |
| C-20 | one individualized compound I | 2-(2-(3-(2,6-dichlorophenyl)- |
| | | 1-methyl- |
| | | allylideneaminooxymethyl)- |
| | | phenyl)-2-methoxyimino- |
| | | N-methyl-acetamide |
| C-21 | one individualized compound I | Benalaxyl |
| C-22 | one individualized compound I | Benalaxyl-M |
| C-23 | one individualized compound I | Benodanil |
| C-24 | one individualized compound I | Benzovindiflupyr |
| C-25 | one individualized compound I | Bixafen |
| C-26 | one individualized compound I | Boscalid |
| C-27 | one individualized compound I | Carboxin |
| C-28 | one individualized compound I | Fenfuram |
| C-29 | one individualized compound I | Fenhexamid |
| C-30 | one individualized compound I | Flutolanil |
| C-31 | one individualized compound I | Fluxapyroxad |
| C-32 | one individualized compound I | Furametpyr |
| C-33 | one individualized compound I | Isopyrazam |
| C-34 | one individualized compound I | Isotianil |
| C-35 | one individualized compound I | Kiralaxyl |
| C-36 | one individualized compound I | Mepronil |
| C-37 | one individualized compound I | Metalaxyl |
| C-38 | one individualized compound I | Metalaxyl-M |
| C-39 | one individualized compound I | Ofurace |
| C-40 | one individualized compound I | Oxadixyl |
| C-41 | one individualized compound I | Oxycarboxin |
| C-42 | one individualized compound I | Penflufen |
| C-43 | one individualized compound I | Penthiopyrad |
| C-44 | one individualized compound I | Sedaxane |
| C-45 | one individualized compound I | Tecloftalam |
| C-46 | one individualized compound I | Thifluzamide |
| C-47 | one individualized compound I | Tiadinil |
| C-48 | one individualized compound I | 2-Amino-4-methyl- |
| | | thiazole-5-carboxylic |
| | | acid anilide |
| C-49 | one individualized compound I | N-(4′- |
| | | trifluoromethylthiobiphenyl- |
| | | 2-yl)-3-difluoromethyl-1- |
| | | methyl-1H-pyrazole-4- |
| | | carboxamide |
| C-50 | one individualized compound I | N-(2-(1,3,3-trimethyl- |
| | | butyl)-phenyl)-1,3-dimethyl- |
| | | 5-fluoro-1H-pyrazole-4- |
| | | carboxamide |
| C-51 | one individualized compound I | 3-(difluoromethyl)-1-methyl- |
| | | N-(1,1,3-trimethylindan-4- |
| | | yl)pyrazole-4-carboxamide |
| C-52 | one individualized compound I | 3-(trifluoromethyl)-1-methyl- |
| | | N-(1,1,3-trimethylindan-4- |
| | | yl)pyrazole-4-carboxamide |
| C-53 | one individualized compound I | 1,3-dimethyl-N-(1,1,3- |
| | | trimethylindan-4-yl) |
| | | pyrazole-4-carboxamide |
| C-54 | one individualized compound I | 3-(trifluoromethyl)-1,5- |
| | | dimethyl-N-(1,1,3- |
| | | trimethylindan-4-yl) |
| | | pyrazole-4-carboxamide |
| C-55 | one individualized compound I | 3-(difluoromethyl)-1,5- |
| | | dimethyl-N-(1,1,3- |
| | | trimethylindan-4-yl) |
| | | pyrazole-4-carboxamide |
| C-56 | one individualized compound I | 1,3,5-trimethyl-N-(1,1,3- |
| | | trimethylindan-4-yl) |
| | | pyrazole-4-carboxamide |
| C-57 | one individualized compound I | Dimethomorph |
| C-58 | one individualized compound I | Flumorph |
| C-59 | one individualized compound I | Pyrimorph |
| C-60 | one individualized compound I | Flumetover |
| C-61 | one individualized compound I | Fluopicolide |
| C-62 | one individualized compound I | Fluopyram |
| C-63 | one individualized compound I | Zoxamide |
| C-64 | one individualized compound I | Carpropamid |
| C-65 | one individualized compound I | Diclocymet |
| C-66 | one individualized compound I | Mandipropamid |
| C-67 | one individualized compound I | Oxytetracyclin |
| C-68 | one individualized compound I | Silthiofam |
| C-69 | one individualized compound I | N-(6-methoxy-pyridin-3-yl) |
| | | cyclopropanecarboxylic |
| | | acid amide |
| C-70 | one individualized compound I | Azaconazole |
| C-71 | one individualized compound I | Bitertanol |
| C-72 | one individualized compound I | Bromuconazole |
| C-73 | one individualized compound I | Cyproconazole |
| C-74 | one individualized compound I | Difenoconazole |
| C-75 | one individualized compound I | Diniconazole |
| C-76 | one individualized compound I | Diniconazole-M |
| C-77 | one individualized compound I | Epoxiconazole |
| C-78 | one individualized compound I | Fenbuconazole |
| C-79 | one individualized compound I | Fluquinconazole |
| C-80 | one individualized compound I | Flusilazole |
| C-81 | one individualized compound I | Flutriafol |
| C-82 | one individualized compound I | Hexaconazol |
| C-83 | one individualized compound I | Imibenconazole |
| C-84 | one individualized compound I | Ipconazole |
| C-85 | one individualized compound I | Metconazole |
| C-86 | one individualized compound I | Myclobutanil |
| C-87 | one individualized compound I | Oxpoconazol |
| C-88 | one individualized compound I | Paclobutrazol |
| C-89 | one individualized compound I | Penconazole |
| C-90 | one individualized compound I | Propiconazole |
| C-91 | one individualized compound I | Prothioconazole |
| C-92 | one individualized compound I | Simeconazole |
| C-93 | one individualized compound I | Tebuconazole |
| C-94 | one individualized compound I | Tetraconazole |
| C-95 | one individualized compound I | Triadimefon |
| C-96 | one individualized compound I | Triadimenol |
| C-97 | one individualized compound I | Triticonazole |
| C-98 | one individualized compound I | Uniconazole |
| C-99 | one individualized compound I | 1-[rel-(2S;3R)-3-(2- |
| | | chlorophenyl)-2-(2,4- |
| | | difluorophenyl)- |
| | | oxiranylmethyl]-5- |
| | | thiocyanato-1H-[1,2, |
| | | 4]triazole, |
| C-100 | one individualized compound I | 2-[rel-(2S;3R)-3-(2- |
| | | chlorophenyl)-2-(2, |
| | | 4-difluorophenyl)- |
| | | oxiranylmethyl]-2H- |
| | | [1,2,4]triazole-3-thiol |
| C-101 | one individualized compound I | Cyazofamid |
| C-102 | one individualized compound I | Amisulbrom |
| C-103 | one individualized compound I | Imazalil |
| C-104 | one individualized compound I | Imazalil-sulfate |
| C-105 | one individualized compound I | Pefurazoate |
| C-106 | one individualized compound I | Prochloraz |
| C-107 | one individualized compound I | Triflumizole |
| C-108 | one individualized compound I | Benomyl |
| C-109 | one individualized compound I | Carbendazim |
| C-110 | one individualized compound I | Fuberidazole |
| C-111 | one individualized compound I | Thiabendazole |
| C-112 | one individualized compound I | Ethaboxam |
| C-113 | one individualized compound I | Etridiazole |
| C-114 | one individualized compound I | Hymexazole |
| C-115 | one individualized compound I | 2-(4-Chloro-phenyl)- |
| | | N-[4-(3,4-dimeth- |
| | | oxy-phenyl)-isoxazol- |
| | | 5-yl]-2-prop-2-yn- |
| | | yloxy-acetamide |
| C-116 | one individualized compound I | Fluazinam |
| C-117 | one individualized compound I | Pyrifenox |
| C-118 | one individualized compound I | 3-[5-(4-Chloro-phenyl)- |
| | | 2,3-dimethyl- |
| | | isoxazolidin-3-yl]- |
| | | pyridine (Pyrisoxazole) |
| C-119 | one individualized compound I | 3-[5-(4-Methyl-phenyl)- |
| | | 2,3-dimethyl-isoxazolidin- |
| | | 3-yl]-pyridine |
| C-120 | one individualized compound I | Bupirimate |
| C-121 | one individualized compound I | Cyprodinil |
| C-122 | one individualized compound I | 5-Fluorocytosine |
| C-123 | one individualized compound I | 5-Fluoro-2-(p- |
| | | tolylmethoxy)pyrimidin- |
| | | 4-amine |
| C-124 | one individualized compound I | 5-Fluoro-2-(4- |
| | | fluorophenylmethoxy)- |
| | | pyrimidin-4-amine |
| C-125 | one individualized compound I | Diflumetorim |
| C-126 | one individualized compound I | (5,8-Difluoroquinazolin- |
| | | 4-yl)-{2-[2-fluoro-4-(4- |
| | | trifluoromethylpyridin-2- |
| | | yloxy)-phenyl]-ethyl}-amine |
| C-127 | one individualized compound I | Fenarimol |
| C-128 | one individualized compound I | Ferimzone |
| C-129 | one individualized compound I | Mepanipyrim |
| C-130 | one individualized compound I | Nitrapyrin |
| C-131 | one individualized compound I | Nuarimol |
| C-132 | one individualized compound I | Pyrimethanil |
| C-133 | one individualized compound I | Triforine |
| C-134 | one individualized compound I | Fenpiclonil |
| C-135 | one individualized compound I | Fludioxonil |
| C-136 | one individualized compound I | Aldimorph |
| C-137 | one individualized compound I | Dodemorph |
| C-138 | one individualized compound I | Dodemorph-acetate |
| C-139 | one individualized compound I | Fenpropimorph |
| C-140 | one individualized compound I | Tridemorph |
| C-141 | one individualized compound I | Fenpropidin |
| C-142 | one individualized compound I | Fluoroimid |
| C-143 | one individualized compound I | Iprodione |
| C-144 | one individualized compound I | Procymidone |
| C-145 | one individualized compound I | Vinclozolin |
| C-146 | one individualized compound I | Famoxadone |
| C-147 | one individualized compound I | Fenamidone |
| C-148 | one individualized compound I | Flutianil |
| C-149 | one individualized compound I | Octhilinone |
| C-150 | one individualized compound I | Probenazole |
| C-151 | one individualized compound I | Fenpyrazamine |
| C-152 | one individualized compound I | Acibenzolar-S-methyl |
| C-153 | one individualized compound I | Ametoctradin |
| C-154 | one individualized compound I | Amisulbrom |
| C-155 | one individualized compound I | [(3S,6S,7R,8R)-8-benzyl- |
| | | 3-[(3-isobutyryloxymethoxy- |
| | | 4-methoxypyridine- |
| | | 2-carbonyl)amino]-6-methyl- |
| | | 4,9-dioxo-[1,5]dioxonan-7-yl] |
| | | 2-methylpropanoate |
| C-156 | one individualized compound I | [(3S,6S,7R,8R)-8-benzyl- |
| | | 3-[(3-acetoxy-4-methoxy- |
| | | pyridine-2-carbonyl)amino]- |
| | | 6-methyl-4,9-dioxo-1,5- |
| | | dioxonan-7-yl]2- |
| | | methylpropanoate |
| C-157 | one individualized compound I | [(3S,6S,7R,8R)-8-benzyl- |
| | | 3-[[3-(acetoxymethoxy)- |
| | | 4-methoxy-pyridine- |
| | | 2-carbonyl]amino]-6- |
| | | methyl-4,9-dioxo-1,5- |
| | | dioxonan-7-yl]2- |
| | | methylpropanoate |
| C-158 | one individualized compound I | [(3S,6S,7R,8R)-8- |
| | | benzyl-3-[(3-isobut- |
| | | oxycarbonyloxy-4- |
| | | methoxy-pyridine- |
| | | 2-carbonyl)amino]-6- |
| | | methyl-4,9-dioxo- |
| | | 1,5-dioxonan-7-yl]2- |
| | | methylpropanoate |
| C-159 | one individualized compound I | (3S,6S,7R,8R)-8-benzyl- |
| | | 3-[[3-(1,3-benzodioxol- |
| | | 5-ylmethoxy)-4-methoxy- |
| | | pyridine-2-carbonyl]amino]- |
| | | 6-methyl-4,9-dioxo- |
| | | 1,5-dioxonan-7-yl]2-methyl- |
| | | propanoate |
| C-160 | one individualized compound I | (3S,6S,7R,8R)-3-[[(3- |
| | | hydroxy-4-methoxy-2- |
| | | pyridinyl)carbonyl]amino]- |
| | | 6-methyl-4,9-dioxo-8- |
| | | (phenylmethyl)-1,5- |
| | | dioxonan-7-yl 2- |
| | | methylpropanoate |
| C-161 | one individualized compound I | Anilazin |
| C-162 | one individualized compound I | Blasticidin-S |
| C-163 | one individualized compound I | Captafol |
| C-164 | one individualized compound I | Captan |
| C-165 | one individualized compound I | Chinomethionat |
| C-166 | one individualized compound I | Dazomet |
| C-167 | one individualized compound I | Debacarb |
| C-168 | one individualized compound I | Diclomezine |
| C-169 | one individualized compound I | Difenzoquat, |
| C-170 | one individualized compound I | Difenzoquat-methylsulfate |
| C-171 | one individualized compound I | Fenoxanil |
| C-172 | one individualized compound I | Folpet |
| C-173 | one individualized compound I | Oxolinsaure |
| C-174 | one individualized compound I | Piperalin |
| C-175 | one individualized compound I | Proquinazid |
| C-176 | one individualized compound I | Pyroquilon |
| C-177 | one individualized compound I | Quinoxyfen |
| C-178 | one individualized compound I | Triazoxid |
| C-179 | one individualized compound I | Tricyclazole |
| C-180 | one individualized compound I | 2-Butoxy-6-iodo-3- |
| | | propyl-chromen-4-one |
| C-181 | one individualized compound I | 5-Chloro-1-(4,6- |
| | | dimethoxy-pyrimidin- |
| | | 2-yl)-2-methyl-1H- |
| | | benzoimidazole |
| C-182 | one individualized compound I | 5-Chloro-7-(4-methyl- |
| | | piperidin-1-yl)-6-(2,4,6- |
| | | trifluoro-phenyl)-[1,2,4] |
| | | triazolo[1,5-a]pyrimidine |
| C-183 | one individualized compound I | Ferbam |
| C-184 | one individualized compound I | Mancozeb |
| C-185 | one individualized compound I | Maneb |
| C-186 | one individualized compound I | Metam |
| C-187 | one individualized compound I | Methasulphocarb |
| C-188 | one individualized compound I | Metiram |
| C-189 | one individualized compound I | Propineb |
| C-190 | one individualized compound I | Thiram |
| C-191 | one individualized compound I | Zineb |
| C-192 | one individualized compound I | Ziram |
| C-193 | one individualized compound I | Diethofencarb |
| C-194 | one individualized compound I | Benthiavalicarb |
| C-195 | one individualized compound I | Iprovalicarb |
| C-196 | one individualized compound I | Propamocarb |
| C-197 | one individualized compound I | Propamocarb hydrochlorid |
| C-198 | one individualized compound I | Valifenalate |
| C-199 | one individualized compound I | N-(1-(1-(4-cyanophenyl) |
| | | ethanesulfon-yl)-but-2- |
| | | yl) carbamic acid-(4-fluoro- |
| | | phenyl) ester |
| C-200 | one individualized compound I | Dodine |
| C-201 | one individualized compound I | Dodine free base |
| C-202 | one individualized compound I | Guazatine |
| C-203 | one individualized compound I | Guazatine-acetate |
| C-204 | one individualized compound I | Iminoctadine |
| C-205 | one individualized compound I | Iminoctadine-triacetate |
| C-206 | one individualized compound I | Iminoctadine-tris(albesilate) |
| C-207 | one individualized compound I | Kasugamycin |
| C-208 | one individualized compound I | Kasugamycin- |
| | | hydrochloride-hydrate |
| C-209 | one individualized compound I | Polyoxine |
| C-210 | one individualized compound I | Streptomycin |
| C-211 | one individualized compound I | Validamycin A |
| C-212 | one individualized compound I | Binapacryl |
| C-213 | one individualized compound I | Dicloran |
| C-214 | one individualized compound I | Dinobuton |
| C-215 | one individualized compound I | Dinocap |
| C-216 | one individualized compound I | Nitrothal-isopropyl |
| C-217 | one individualized compound I | Tecnazen |
| C-218 | one individualized compound I | Fentin salts |
| C-219 | one individualized compound I | Dithianon |
| C-220 | one individualized compound I | 2,6-dimethyl-1H,5H-[1,4] |
| | | dithiino[2,3-c:5,6-c′] |
| | | dipyrrole-1,3,5,7(2H,6H)- |
| | | tetraone |
| C-221 | one individualized compound I | Isoprothiolane |
| C-222 | one individualized compound I | Edifenphos |
| C-223 | one individualized compound I | Fosetyl, Fosetyl-aluminium |
| C-224 | one individualized compound I | Iprobenfos |
| C-225 | one individualized compound I | Phosphorous acid (H₃PO₃) |
| | | and derivatives |
| C-226 | one individualized compound I | Pyrazophos |
| C-227 | one individualized compound I | Tolclofos-methyl |
| C-228 | one individualized compound I | Chlorothalonil |
| C-229 | one individualized compound I | Dichlofluanid |
| C-230 | one individualized compound I | Dichlorophen |
| C-231 | one individualized compound I | Flusulfamide |
| C-232 | one individualized compound I | Hexachlorbenzene |
| C-233 | one individualized compound I | Pencycuron |
| C-234 | one individualized compound I | Pentachlorophenol and salts |
| C-235 | one individualized compound I | Phthalide |
| C-236 | one individualized compound I | Quintozene |
| C-237 | one individualized compound I | Thiophanate Methyl |
| C-238 | one individualized compound I | Tolylfluanid |
| C-239 | one individualized compound I | N-(4-chloro-2-nitro- |
| | | phenyl)-N-ethyl-4- |
| | | methyl-benzenesulfonamide |
| C-240 | one individualized compound I | Bordeaux composition |
| C-241 | one individualized compound I | Copper acetate |
| C-242 | one individualized compound I | Copper hydroxide |
| C-243 | one individualized compound I | Copper oxychloride |
| C-244 | one individualized compound I | basic Copper sulfate |
| C-245 | one individualized compound I | Sulfur |
| C-246 | one individualized compound I | Biphenyl |
| C-247 | one individualized compound I | Bronopol |
| C-248 | one individualized compound I | Cyflufenamid |
| C-249 | one individualized compound I | Cymoxanil |
| C-250 | one individualized compound I | Diphenylamin |
| C-251 | one individualized compound I | Metrafenone |
| C-252 | one individualized compound I | Pyriofenone |
| C-253 | one individualized compound I | Mildiomycin |
| C-254 | one individualized compound I | Oxin-copper |
| C-255 | one individualized compound I | Oxathiapiprolin |
| C-256 | one individualized compound I | Prohexadione calcium |
| C-257 | one individualized compound I | Spiroxamine |
| C-258 | one individualized compound I | Tebufloquin |
| C-259 | one individualized compound I | Tolylfluanid |
| C-260 | one individualized compound I | N-(Cyclopropyl- |
| | | methoxyimino-(6- |
| | | difluoromethoxy-2, |
| | | 3-difluoro-phenyl)- |
| | | methyl)-2-phenyl |
| | | acetamide |
| C-261 | one individualized compound I | N′-(4-(4-chloro-3- |
| | | trifluoromethyl- |
| | | phenoxy)-2,5-dimethyl- |
| | | phenyl)-N-ethyl- |
| | | N-methyl formamidine |
| C-262 | one individualized compound I | N′-(4-(4-fluoro-3- |
| | | trifluoromethyl- |
| | | phenoxy)-2,5-dimethyl- |
| | | phenyl)-N-ethyl- |
| | | N-methyl formamidine |
| C-263 | one individualized compound I | N′-(2-methyl-5- |
| | | trifluoromethyl-4-(3-tri- |
| | | methylsilanyl-propoxy)- |
| | | phenyl)-N-ethyl- |
| | | N-methyl formamidine |
| C-264 | one individualized compound I | N′-(5-difluoromethyl-2- |
| | | methyl-4-(3-tri- |
| | | methylsilanyl-propoxy)- |
| | | phenyl)-N-ethyl- |
| | | N-methyl formamidine |
| C-265 | one individualized compound I | Methoxy-acetic acid 6- |
| | | tert-butyl-8-fluoro-2,3- |
| | | dimethyl-quinolin-4-yl ester |
| C-266 | one individualized compound I | Bacillus subtilis NRRL |
| | | No. B-21661 |
| C-267 | one individualized compound I | Bacillus pumilus NRRL |
| | | No. B-30087 |
| C-268 | one individualized compound I | Ulocladium oudemansii |
| C-269 | one individualized compound I | Carbaryl |
| C-270 | one individualized compound I | Carbofuran |
| C-271 | one individualized compound I | Carbosulfan |
| C-272 | one individualized compound I | Methomylthiodicarb |
| C-273 | one individualized compound I | Bifenthrin |
| C-274 | one individualized compound I | Cyfluthrin |
| C-275 | one individualized compound I | Cypermethrin |
| C-276 | one individualized compound I | alpha-Cypermethrin |
| C-277 | one individualized compound I | zeta-Cypermethrin |
| C-278 | one individualized compound I | Deltamethrin |
| C-279 | one individualized compound I | Esfenvalerate |
| C-280 | one individualized compound I | Lambda-cyhalothrin |
| C-281 | one individualized compound I | Permethrin |
| C-282 | one individualized compound I | Tefluthrin |
| C-283 | one individualized compound I | Diflubenzuron |
| C-284 | one individualized compound I | Flufenoxuron |
| C-285 | one individualized compound I | Lufenuron |
| C-286 | one individualized compound I | Teflubenzuron |
| C-287 | one individualized compound I | Spirotetramate |
| C-288 | one individualized compound I | Clothianidin |
| C-289 | one individualized compound I | Dinotefuran |
| C-290 | one individualized compound I | Imidacloprid |
| C-291 | one individualized compound I | Thiamethoxam |
| C-292 | one individualized compound I | Flupyradifurone |
| C-293 | one individualized compound I | Acetamiprid |
| C-294 | one individualized compound I | Thiacloprid |
| C-295 | one individualized compound I | Endosulfan |
| C-296 | one individualized compound I | Fipronil |
| C-297 | one individualized compound I | Abamectin |
| C-298 | one individualized compound I | Emamectin |
| C-299 | one individualized compound I | Spinosad |
| C-300 | one individualized compound I | Spinetoram |
| C-301 | one individualized compound I | Hydramethylnon |
| C-302 | one individualized compound I | Chlorfenapyr |
| C-303 | one individualized compound I | Fenbutatin oxide |
| C-304 | one individualized compound I | Indoxacarb |
| C-305 | one individualized compound I | Metaflumizone |
| C-306 | one individualized compound I | Flonicamid |
| C-307 | one individualized compound I | Lubendiamide |
| C-308 | one individualized compound I | Chlorantraniliprole |
| C-309 | one individualized compound I | Cyazypyr (HGW86) |
| C-310 | one individualized compound I | Cyflumetofen |
| C-311 | one individualized compound I | Acetochlor |
| C-312 | one individualized compound I | Dimethenamid |
| C-313 | one individualized compound I | metolachlor |
| C-314 | one individualized compound I | Metazachlor |
| C-315 | one individualized compound I | Glyphosate |
| C-316 | one individualized compound I | Glufosinate |
| C-317 | one individualized compound I | Sulfosate |
| C-318 | one individualized compound I | Clodinafop |
| C-319 | one individualized compound I | Fenoxaprop |
| C-320 | one individualized compound I | Fluazifop |
| C-321 | one individualized compound I | Haloxyfop |
| C-322 | one individualized compound I | Paraquat |
| C-323 | one individualized compound I | Phenmedipham |
| C-324 | one individualized compound I | Clethodim |
| C-325 | one individualized compound I | Cycloxydim |
| C-326 | one individualized compound I | Profoxydim |
| C-327 | one individualized compound I | Sethoxydim |
| C-328 | one individualized compound I | Tepraloxydim |
| C-329 | one individualized compound I | Pendimethalin |
| C-330 | one individualized compound I | Prodiamine |
| C-331 | one individualized compound I | Trifluralin |
| C-332 | one individualized compound I | Acifluorfen |
| C-333 | one individualized compound I | Bromoxynil |
| C-334 | one individualized compound I | Imazamethabenz |
| C-335 | one individualized compound I | Imazamox |
| C-336 | one individualized compound I | Imazapic |
| C-337 | one individualized compound I | Imazapyr |
| C-338 | one individualized compound I | Imazaquin |
| C-339 | one individualized compound I | Imazethapyr |
| C-340 | one individualized compound I | 2,4-Dichloro- |
| | | phenoxyacetic acid (2,4-D) |
| C-341 | one individualized compound I | Chloridazon |
| C-342 | one individualized compound I | Clopyralid |
| C-343 | one individualized compound I | Fluroxypyr |
| C-344 | one individualized compound I | Picloram |
| C-345 | one individualized compound I | Picolinafen |
| C-346 | one individualized compound I | Bensulfuron |
| C-347 | one individualized compound I | Chlorimuron-ethyl |
| C-348 | one individualized compound I | Cyclosulfamuron |
| C-349 | one individualized compound I | Iodosulfuron |
| C-350 | one individualized compound I | Mesosulfuron |
| C-351 | one individualized compound I | Metsulfuron-methyl |
| C-352 | one individualized compound I | Nicosulfuron |
| C-353 | one individualized compound I | Rimsulfuron |
| C-354 | one individualized compound I | Triflusulfuron |
| C-355 | one individualized compound I | Atrazine |
| C-356 | one individualized compound I | Hexazinone |
| C-357 | one individualized compound I | Diuron |
| C-358 | one individualized compound I | Florasulam |
| C-359 | one individualized compound I | Pyroxasulfone |
| C-360 | one individualized compound I | Bentazone |
| C-361 | one individualized compound I | Cinidon-ethyl |
| C-362 | one individualized compound I | Cinmethylin |
| C-363 | one individualized compound I | Dicamba |
| C-364 | one individualized compound I | Diflufenzopyr |
| C-365 | one individualized compound I | Quinclorac |
| C-366 | one individualized compound I | Quinmerac |
| C-367 | one individualized compound I | Mesotrione |
| C-368 | one individualized compound I | Saflufenacil |
| C-369 | one individualized compound I | Topramezone |

The active substances referred to as component 2, their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657).

The compositions of active substances can be prepared as compositions comprising besides the active ingredients at least one inert ingredient by usual means, e. g. by the means given for the compositions of compounds I.

Concerning usual ingredients of such compositions reference is made to the explanations given for the compositions containing compounds I.

The compositions of active substances according to the present invention are suitable as fungicides, as are the compounds of formula I. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is referred to the explanations regarding the fungicidal activity of the compounds and the compositions containing compounds I, respectively.

### SYNTHESIS EXAMPLES

With due modification of the starting compounds, the procedures shown in the synthesis examples below were used to obtain further compounds I. The resulting compounds, together with physical data, are listed in Table I below.

### I. Synthesis Example

### 1) Synthese 6-Methyl-5H-Pyrrolo[3″,4″:5,6][1,4]dithiino-[2,3-C]isothiazole-3-carbonitrile-5,7(6H)-dione

To the mixture of 0.69 g 3,4-dichloro-1-methyl-pyrrole-2,5-dione in 20 ml DMF added 1 g disodium 5-cyanoisothiazoledithiolate and the mixture stirred 3 h at 50° C. To the reaction mixture added 60 ml MtBE and washed with water. The organic phase was evaporated to dryness under reduced pressure and the crude product was purified by column chromatography using cyclohexan/MtBE (9:1) as eluent to yield 0.89 g of the titled product.

¹H-NMR (CDCl₃): δ=3.1 (s, 3H) ppm

MP: 203-211° C.

According to the synthesis example compounds of the formula I were prepared as listed in the Table 1:

TABLE 1
Compounds of formula I with physical data (melting point [° C.]; ¹H-NMR [CDCl₃,
δ].

| No. | R1 | R2 | k | l | m.p [° C.]; ¹H-NMR [CDCl₃, δ]. |
|---|---|---|---|---|---|
| 1.1 | CH₃ | CN | 0 | 0 | 206° C. |
| 1.2 | CH₂CH₃ | CN | 0 | 0 | 206° C. |
| 1.3 | CH₂CH₂CH═CH₂ | CN | 0 | 0 | 189° C. |
| 1.4 | CH(CH₃)₂ | CN | 0 | 0 | 170° C. |
| 1.5 | CH₂CH₂CH₃ | CN | 0 | 0 | 175° C. |
| 1.6 | CH₂CH═CH₂ | CN | 0 | 0 | 184° C. |
| 1.7 | cyclopentan | CN | 0 | 0 | 178° C. |
| 1.8 | CH₂CF₃ | CN | 0 | 0 | 242° C. |
| 1.9 | cyclopropan | CN | 0 | 0 | 243° C. |
| 1.10 | CH(CH₃)CH(OCH₃)₂ | CN | 0 | 0 | 119° C. |
| 1.11 | OCH₂CH₃ | CN | 0 | 0 | 158° C. |
| 1.12 | OCH₃ | CN | 0 | 0 | 210° C. |
| 1.13 | CH₂CH₃ | C(═O)NH₂ | 0 | 0 | 1H-NMR: 8.5 (s); 8.2 (s); 3.5 (q); 1.0 (t) |
| 1.14 | H | CN | 0 | 0 | 290° C. |
| 1.15 | CH₂CH₃ | C(═O)OCH₃ | 0 | 0 | 1H-NMR: 1.1 (s); 3.45 (q); 3.9 |
| | | | | | (s) |

### II. Examples of the Action Against Harmful Fungi

The fungicidal action of the compounds of the formula I was demonstrated by the following experiments:

A) Green House

The spray solutions were prepared in several steps:

The stock solution were prepared: a mixture of acetone and/or dimethylsulfoxide and the wetting agent/emulsifier Wettol, which is based on ethoxylated alkylphenoles, in a relation (volume) solvent-emulsifier of 99 to 1 was added to 25 mg of the compound to give a total of 5 ml.

Water was then added to total volume of 100 ml.

This stock solution was diluted with the described solvent-emulsifier-water mixture to the given concentration.

### Example 1

### Protective Control of Soy Bean Rust on Soy Beans Caused by Phakopsora pachyrhizi

Leaves of pot-grown soy bean seedlings were sprayed to run-off with an aqueous suspension, containing the concentration of active ingredient or their mixture as described below. The plants were allowed to air-dry. The trial plants were cultivated for 1 day in a greenhouse chamber at 23-27° C. and a relative humidity between 60 and 80%. Then the plants were inoculated with spores of Phakopsora pachyrhizi. To ensure the success the artificial inoculation, the plants were transferred to a humid chamber with a relative humidity of about 95% and 20 to 24° C. for 24 h. The trial plants were cultivated for fourteen days in a greenhouse chamber at 23-27° C. and a relative humidity between 60 and 80%. The extent of fungal attack on the leaves was visually assessed as % diseased leaf area.

In this test, the plants which had been treated with 500 ppm of the active substance from examples 1.1 and 1.2 showed an infection of less than or equal to 7% whereas the untreated plants were 90% infected.

### Example 2

### Control of Late Blight on Tomatoes Caused by Phytophthora infestans

Young seedlings of tomato plants were grown in pots. These plants were sprayed to run-off with an aqueous suspension, containing the concentration of active ingredient or their mixture mentioned in the table below. The next day, the treated plants were inoculated with an aqueous suspension of sporangia of Phytophthora infestans. After inoculation, the trial plants were immediately transferred to a humid chamber. After 6 days at 18 to 20° C. and a relative humidity close to 100% the extent of fungal attack on the leaves was visually assessed as % diseased leaf area.

In this test, the plants which had been treated with 500 ppm of the active substance from examples 1.1 and 1.2 showed an infection of less than or equal to 0% whereas the untreated plants were 90% infected.

### Example 3

### Preventative Fungicidal Control of Early Blight on Tomatoes (Alternaria solani)

Young seedlings of tomato plants were grown in pots. These plants were sprayed to run-off with an aqueous suspension, containing the concentration of active ingredient or mixture mentioned in the table below. The next day, the treated plants were inoculated with an aqueous suspension of Alternaria solani. Then the trial plants were immediately transferred to a humid chamber. After 5 days at 18 to 20° C. and a relative humidity close to 100%, the extent of fungal attack on the leaves was visually assessed as % diseased leaf area.

In this test, the plants which had been treated with 500 ppm of the active substance from examples 1.1 and 1.2 showed an infection of less than or equal to 5% and 1% respectively whereas the untreated plants were 90% infected.

### Example 4

### Preventative Control of Leaf Blotch on Wheat Caused by Septoria tritici

Leaves of pot-grown wheat seedling were sprayed to run-off with an aqueous suspension of the active compound or their mixture, prepared as described. The plants were allowed to air-dry. At the following day the plants were inoculated with an aqueous spore suspension of Septoria tritici. Then the trial plants were immediately transferred to a humid chamber at 18-22° C. and a relative humidity close to 100%. After 4 days the plants were transferred to a chamber with 18-22° C. and a relative humidity close to 70%. After 4 weeks the extent of fungal attack on the leaves was visually assessed as % diseased leaf area.

In this test, the plants which had been treated with 500 ppm of the active substance from examples 1.1 and 1.2 showed an infection of less than or equal to 3% and 10% respectively whereas the untreated plants were 90% infected.

### Example 5

### Protective Control of Soy Bean Rust on Soy Beans Caused by Phakopsora pachyrhizi

Leaves of pot-grown soy bean seedlings were sprayed to run-off with an aqueous suspension, containing the concentration of active ingredient or their mixture as described below. The plants were allowed to air-dry. The trial plants were cultivated for 1 day in a greenhouse chamber at 23-27° C. and a relative humidity between 60 and 80%. Then the plants were inoculated with spores of Phakopsora pachyrhizi. To ensure the success the artificial inoculation, the plants were transferred to a humid chamber with a relative humidity of about 95% and 20 to 24° C. for 24 h. The trial plants were cultivated for fourteen days in a greenhouse chamber at 23-27° C. and a relative humidity between 60 and 80%. The extent of fungal attack on the leaves was visually assessed as % diseased leaf area.

In this test, the plants which had been treated with 500 ppm of the active substance from examples 1.1, 1.2, 1.4 and 1.6 showed an infection of less than or equal to 1%, 3% and 10% respectively whereas the untreated plants were 90% infected.

### Example 6

### Preventative Control of Leaf Blotch on Wheat Caused by Septoria tritici

Leaves of pot-grown wheat seedling were sprayed to run-off with an aqueous suspension of the active compound or their mixture, prepared as described. The plants were allowed to air-dry. At the following day the plants were inoculated with an aqueous spore suspension of Septoria tritici. Then the trial plants were immediately transferred to a humid chamber at 18-22° C. and a relative humidity close to 100%. After 4 days the plants were transferred to a chamber with 18-22° C. and a relative humidity close to 70%. After 4 weeks the extent of fungal attack on the leaves was visually assessed as % diseased leaf area.

In this test, the plants which had been treated with 500 ppm of the active substance from examples 1.1, 1.2, 1.4, 1.5, 1.6, 1.11 and 1.12 showed an infection of less between 7% to 15% whereas the untreated plants were 90% infected.

1-14. (canceled)

15. A compound of the formula I 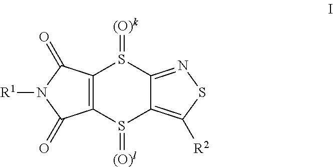
in which
k is 0, 1 or 2; or
l is 0, 1 or 2; or
R¹ is H, OH, halogen, CN, NO₂; or
C₁-C₁₀-alkyl, C₁-C₁₀-alkoxy, C₁-C₁₀-haloalkyl, C₂-C₁₀-alkenyl, C₂-C₁₀-alkynyl, C₃-C₁₀-cycloalkyl, C₃-C₁₀-halocycloalkyl, C₃-C₁₀-cycloalkenyl; or
phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein
the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₂-C₁₀-alkenyl, C₂-C₁₀-alkynyl, C₁-C₁₀-alkoxy, C₁-C₁₀-haloalkoxy, OA³, —C(═O)A⁴, —C(═O)OA⁴
wherein OA³, —C(═O)A⁴ are as defined below; or
NA¹A² where
A¹ and A² independently of one another are hydrogen, C₁-C₁₀-alkyl, C₁-C₁₀-haloalkyl, C₃-C₁₀-cycloalkyl, C₃-C₁₀-halocycloalkyl, C₁-C₁₀-alkoxy-C₁-C₁₀-alkyl, amino-C₁-C₁₀-alkyl, or
independently of one another are phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein
the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy; or
OA³ where
A³ is C₁-C₁₀-alkyl, C₁-C₄-alkylcarbonyl, C₁-C₁₀-haloalkyl, C₂-C₆-alkenyl, C₂-C₆-alkynyl, C₃-C₁₀-cycloalkyl, C₃-C₁₀-halocycloalkyl, C₃-C₆-cycloalkenyl, phenyl, benzyl, naphthyl or a saturated, partially unsaturated or aromatic 5-, 6-, 7-, 8, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members, wherein
the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy; or
a radical of the formula —C(═O)A⁴, —C(═O)OA⁴, —OC(═O)A⁴, —NA⁴C(═O)A⁴, —NA⁴C(═O)OA⁴, —N═OA⁴ where
A⁴ is hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO, SO or SO₂ groups as ring members,
wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy; or
a radical of the formula —S(O)ₙA⁵,—OS(O)ₙA⁵, —NA⁵S(O)ₙA⁵ where
n=0, 1, 2 and
A⁵ independently of one another are hydrogen, hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated or unsaturated aromatic or non-aromatic 5-, 6-, 7-, 8, 9- or 10-membered heterocycle, where the heterocycle contains 1, 2, 3 or 4 heteroatoms selected from the group consisting of O, N and S as ring members and may furthermore contain one or two CO groups as ring members,
wherein the above mentioned groups may carry one, two, three or four identical or different substituents selected from the group consisting of halogen, hydroxyl, cyano, nitro, NH₂, C₁-C₁₀-alkyl, C₁-C₁₀-haloalkyl, C₁-C₁₀-alkoxy, C₁-C₁₀-haloalkoxy
R² is H, halogen, CN, NO₂; or
NA¹A²
where
A¹ and A² independently of one another are hydrogen, C₁-C₁₀-alkyl, C₁-C₁₀-haloalkyl, C₃-C₁₀-cycloalkyl, C₃-C₁₀-halocycloalkyl, C₁-C₁₀-alkoxy-C₁-C₁₀-alkyl, amino-C₁-C₁₀-alkyl,
wherein
the amino group is substituted by B¹ and B² which are independently of one another hydrogen, C₁-C₁₀-alkyl, C₁-C₁₀-haloalkyl, C₃-C₁₀-cycloalkyl, C₃-C₁₀-halocycloalkyl or B¹ and B² together with the N atom to which these radicals are attached may also form a five-, six-, seven-, eight-, nine- or ten-membered saturated or partially unsaturated ring which, in addition to carbon atoms, may contain one, two or three heteroatoms from the group consisting of O, N and S as ring members;
or
independently of one another are phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO groups as ring members,
wherein the above mentioned groups may carry one, two, three or four identical or different substituents selected from the group consisting of halogen, hydroxyl, cyano, nitro, NH₂, mono-(C₁-C₁₀-alkyl)amino, di-(C₁-C₁₀-alkyl)amino, C₁-C₁₀-alkyl, C₁-C₁₀-haloalkyl, C₁-C₁₀-alkoxy, C₁-C₁₀-haloalkoxy; or
a radical of the formula
—C(═O)A⁴, —C(═O)OA⁴, —NA⁴C(═O)A⁴, —N═OA⁴,
where
A⁴ independently of one another are hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkyl)amino, di-(C₁-C₁₀-alkyl)amino, phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO groups as ring members,
wherein the above mentioned groups may carry one, two, three or four identical or different substituents selected from the group consisting of halogen, hydroxyl, cyano, nitro, NH₂, mono-(C₁-C₁₀-alkyl)amino, di-(C₁-C₁₀-alkyl)amino, C₁-C₁₀-haloalkyl, C₁-C₁₀-alkoxy, C₁-C₁₀-haloalkoxy; or
—S(O)ₙA⁵, OS(O)ₙA⁵, —NA⁵S(O)ₙA⁵
where
n=0, 1, 2
A⁵ independently of one another are hydrogen, C₁-C₁₀-alkyl, C₁-C₁₀-haloalkyl, C₃-C₁₀-cycloalkyl, C₃-C₁₀-halocycloalkyl, NH₂, mono-(C₁-C₁₀-alkylamino, di-(C₁-C₁₀-alkyl)amino, phenyl, benzyl, naphthyl or a saturated, partially unsaturated or aromatic 5-, 6-, 7-, 8-, 9- or 10-membered heterocycle, where the heterocycle contains 1, 2, 3 or 4 heteroatoms selected from the group consisting of O, N and S as ring members and may furthermore contain one or two CO groups as ring members,
wherein the above mentioned groups may carry one, two, three or four identical or different substituents selected from the group consisting of halogen, hydroxyl, cyano, nitro, NH₂, mono-(C₁-C₁₀-alkyl)amino, di-(C₁-C₁₀-alkyl)amino, C₁-C₁₀-alkyl, C₁-C₁₀-haloalkyl, C₁-C₁₀-alkoxy, C₁-C₁₀-haloalkoxy;
and the N-oxides and the agriculturally acceptable salts thereof.

16. The compound according to claim 15, wherein R² is CN.

17. The compound according to claim 15, wherein R² is NO₂.

18. The compound according to claim 15, wherein R² is —C(═O)A⁴.

19. The compound according to claim 15, wherein R² is —C(═O)OA⁴.

20. The compound according to claim 15, wherein R¹ is C₁-C₁₀-alkyl.

21. The compound according to claim 15, wherein R¹ is C₁-C₁₀-alkoxy.

22. The compound according to claim 15, wherein R¹ is NA¹A².

23. A process for preparing compounds of formula I as defined in claim 15, which comprises reacting a compound of formula III 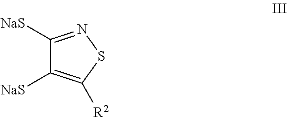
with a compound of the formula IV 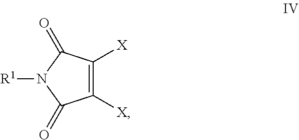
wherein X is halogen,
in the presence of a solvent;
and optionally oxidation of the resulting compound of formula IA 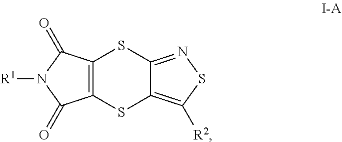
to obtain compounds I.

24. Agrochemical compositions, wherein said composition comprise an auxiliary and at least one compound of formula I, as defined in claim 15, an N-oxide or an agriculturally acceptable salt thereof.

25. The compositions according to claim 10, comprising additionally a further active substance.

26. A method for combating harmful fungi, comprising treating the fungi or the materials, plants, the soil or seeds to be protected against fungal attack with an effective amount of at least one compound of formula I, as defined in claim 15.

27. The method according to claim 26, wherein, in the compound of formula I, R² is CN.

28. The method according to claim 26, wherein, in the compound of formula I, R² is NO₂.

29. The method according to claim 26, wherein, in the compound of formula I, R² is —C(═O)A⁴.

30. The method according to claim 26, wherein, in the compound of formula I, R² is —C(═O)OA⁴.

31. The method according to claim 26, wherein, in the compound of formula I, R¹ is C₁-C₁₀-alkyl.

32. The method according to claim 26, wherein, in the compound of formula I, R¹ is C₁-C₁₀-alkoxy.

33. The method according to claim 26, wherein, in the compound of formula I, R¹ is NA¹A².

34. Seed, coated with at least one compound of the formula I, as defined in claim 15, and/or an agriculturally acceptable salt thereof in an amount of from 0.1 to 10 kg per 100 kg of seed.
